(19) United States
(12) Patent Application Publication
(10) Pub. No.: US 2007/0292355 A1
(43) Pub. Date: Dec. 20, 2007

(54) Anti-infection augmentation foamable compositions and kit and uses thereof

(75) Inventors: Dov Tamarkin, Maccabim (IL); Doron Friedman, Karmei Yosef (IL); Meir Eini, Ness Ziona (IL)

Correspondence Address: WILMERHALE/BOSTON, 60 STATE STREET, BOSTON, MA, 02109, US

(73) Assignee: Foamix Ltd.

(21) Appl. No.: 11/732,547

(22) Filed: Apr. 4, 2007

Related U.S. Application Data
(63) Continuation-in-part of application No. 10/532,618, filed on Dec. 22, 2005.
(63) Continuation-in-part of application No. 10/835,505, filed on Apr. 28, 2004.
(63) Continuation-in-part of application No. 10/922,358, filed on Aug. 20, 2004.
(63) Continuation-in-part of application No. 11/041,921, filed on Jan. 24, 2005.
Application No. 11/041,921 is a continuation-in-part of application No. 10/911,367, filed on Aug. 4, 2004.
(63) Continuation-in-part of application No. 11/448,490, filed on Jun. 7, 2006.
Application No. 11/448,490 is a continuation-in-part of application No. 10/911,367, filed on Aug. 4, 2004.
Application No. 11/448,490 is a continuation-in-part of application No. 10/532,618, filed on Dec. 22, 2005.
(60) Provisional application No. 60/492,385, filed on Aug. 4, 2003.
(60) Provisional application No. 60/530,015, filed on Dec. 16, 2003.
(60) Provisional application No. 60/497,648, filed on Aug. 25, 2003.
(60) Provisional application No. 60/492,385, filed on Aug. 4, 2003.
(60) Provisional application No. 60/429,546, filed on Nov. 29, 2002.
(60) Provisional application No. 60/688,244, filed on Jun. 7, 2005.
(60) Provisional application No. 60/789,186, filed on Apr. 4, 2006.
(60) Provisional application No. 60/861,620, filed on Nov. 29, 2006.
(60) Provisional application No. 60/880,434, filed on Jan. 12, 2007.

(30) Foreign Application Priority Data
Oct. 25, 2002 (IL) 152486

(51) Int. Cl. A61K 9/12; A61K 31/015; A61K 31/415; A61K 31/4164; A61K 31/425; A61K 31/433; A61K 31/44; A61K 31/497; A61P 11/00; A61P 35/00; A61P 31/18; A61P 31/12; A61P 31/10; A61P 27/16; A61P 17/00; A61K 31/505; A61K 31/52; A61K 31/522; A61K 31/551; A61K 31/70; A61K 36/00; A61K 38/00

(52) U.S. Cl. 424/43; 424/725; 514/2; 514/220; 514/253.09; 514/263.37; 514/263.4; 514/27; 514/274; 514/307; 514/361; 514/365; 514/396; 514/406; 514/766

(57) ABSTRACT

Anti-infective foamable composition and kits include a foamable carrier; a therapeutically safe and effective concentration of an anti-infective agent; an augmenting agent selected from the group consisting of a keratolytic agent and a skin penetration enhancer; and a propellant. The composition is housed in a container and upon release is expandable to form a breakable foam. The foamable carrier is selected to generate a foam of good or excellent quality in the presence of the augmenting agent and anti-infective agent. Methods for treating, alleviating or preventing a disorder of the skin, a body cavity or mucosal surface, wherein the disorder involves a fungal, bacterial or viral infection as one of its etiological factors, is described.

### CROSS-REFERENCE TO RELATED APPLICATIONS

This application is a continuation-in-part application of co-pending U.S. patent application Ser. No. 10/532,618, filed on Dec. 22, 2005, which is an application filed under 35 U.S.C. §371 of International Patent Application No. IB03/005527 designating the U.S. and filed on Oct. 24, 2003, which claims the benefit of priority under 35 U.S.C. §119(e) to U.S. Provisional Patent Application No. 60/429,546, filed on Nov. 29, 2002, both entitled “Cosmetic and Pharmaceutical Foam,” and which also claims the benefit of priority under 35 USC§119(a) to Israeli Patent App. No. 152486, filed Oct. 25, 2002; all of which are hereby incorporated in their entirety by reference.

This application is a continuation-in-part of U.S. patent application Ser. No. 10/835,505, filed on Apr. 28, 2004, entitled “Oleaginous Pharmaceutical and Cosmetic Foam,” which claims priority under 35 U.S.C. § 119(e) of U.S. Provisional Patent Application No. 60/530,015, filed on Dec. 16, 2003 and 60/492,385, filed on Aug. 4, 2003, which are hereby incorporated in their entirety by reference.

This application is a continuation-in-part application of co-pending U.S. patent application Ser. No. 10/922,358, filed on Aug. 20, 2004, entitled “Penetrating Pharmaceutical Foam,” which is hereby incorporated in its entirety by reference.

This application is a continuation-in-part application of co-pending U.S. patent application Ser. No. 11/041,921, filed on Jan. 24, 2005, entitled “Kit And Composition Of Imidazole With Enhanced Bioavailability,” which is hereby incorporated in its entirety by reference.

This application is a continuation-in-part application of co-pending U.S. patent application Ser. No. 11/448,490, filed on Jun. 7, 2006, entitled “Antibiotic Kit And Composition And Uses Thereof,” which claims the benefit of priority under 35 U.S.C. § 119(e) to U.S. Provisional Patent Application No. 60/688,244, filed on Jun. 7, 2005, which are hereby incorporated in their entirety by reference.

This application claims the benefit under 35 U.S.C. §119(e) of U.S. Provisional Patent Application No. 60/789,186, filed on Apr. 4, 2006, entitled “Keratolytic Antifungal Foam,” which is hereby incorporated in its entirety by reference.

The application claims the benefit of priority under 35 U.S.C. §119(e) to co-pending U.S. Patent Application No. 60/861,620, filed on Nov. 29, 2006, entitled “Foamable Compositions with Modulating Agents,” which is hereby incorporated in its entirety by reference.

The application claims the benefit of priority under 35 U.S.C. §119(e) to co-pending U.S. Patent Application No. 60/880,434, filed on Jan. 12, 2007, entitled “Hydrophilic or Waterless Vehicle and Foamable Pharmaceutical Compositions,” which is hereby incorporated in its entirety by reference.

### BACKGROUND OF THE INVENTION

The present invention relates to the treatment of skin infections. They can be primary or secondary skin infections.

A secondary skin infection can for example be as a result of damage to the skin, which is then followed by an infection or during or after a primary infection the skin is infected by another organism or is reinfected. The damage can be mechanical or due to an organism or an illness or radiation or chemical exposure or inflammation other means. The primary infection may be fungal, bacterial, viral or in consequence of a parasitic infestation. Parasites can be carriers of various infections or can render the host vulnerable to infection by virtue of the damage they do on the skin. Treatment may be retarded or prevented by the keratin layer. Keratin forms a tough, impervious, waterproof layer that protects skin, stopping unwanted substances in the outside world from penetrating the skin and getting into underlying tissue. Keratin builds up in areas of the body that get exposed to lots of wear and tear and is expressed for example in the form of a callous. As keratin builds up in cells, the cells die and eventually peel, gradually losing small amounts of skin and hair.

Dermatophytic infection of the skin can manifest themselves in different anatomical regions of the body and has been accordingly named. Thus, tinea capitis affects the scalp, tinea barbae—the face, tinea unguium—the nails, tinea manuum—the hands, and tinea cruris—the groin area. Tinea pedis, also known as athlete's foot, is a chronic fungal infection of the feet. Tinea pedis is estimated to be the second most common skin disease in the United States, behind acne, and up to 15% of the population may manifest the disease.

Tinea pedis presents as pruritic, erythematous, inflamed regions on the feet that may be located on the sole (vesicular type) or lateral aspects (moccasin type) of the foot and sometimes between the toes (interdigital type). Three main genera of fungi may cause tinea pedis, Trichophyton, Epidermophyton, and Microsporum. Other, nondermatophtye, fungi like Malassezia furfur, corynebacterium minutissimum, and Candida species may also cause tinea pedis.

According to the known prior art, for simple cases, Athlete's foot is treated locally with antifungal creams, sprays, liquids and powders based on imidazole antifungals such as clotrimazole and miconazole, as well as zinc undecenoate, allylamines, such as terbinafine, and tolnaflate. Fungal infections usually affect the skin because they live off keratin, a protein that makes up skin, hair and nails.

Prior art data has shown that topical antifungal treatment fails to cure about one-third of patients with tinea pedis (Bell-Syer S E, Hart R, Crawford F, Torgerson D J, Young P, Tyrrell W, Williams H, Russell I: A systematic review of oral treatments for fungal infections of the skin of the feet. J Dermatolog Treat 2001, 12:69-74).

In more severe cases, or if an infection is resistant to usual treatment, antifungal pills may be prescribed. It is important to continue the use of the prescribed antifungal creams and to take all the oral medications properly.

### SUMMARY OF THE INVENTION

In certain aspects, the present invention relates to the use of keratolytic agents in combination with an anti-infective agent (e.g., antifungal, antibiotic or antiviral agent or combinations thereof) to help to remove or reduce part of the keratin layer at a target site so as to enable an active agent reach the site of infection so as to have an improved effect. The present invention also relates to the use of penetrating agents having a keratolytic like effect in combination with an anti-infective agent. Keratolytic agents and penetrating agents having a keratolytic effect are referred to herein as “augmenting agents.”

In certain aspects, the present invention further relates to the delivery of keratolytic agents, active agents and penetration enhancers to a target site from a foamed composition.

In certain aspects, the present invention further relates to a kit for the delivery of keratolytic agents, active agents and penetration enhancers to a target site in one or more foamable compositions and foams.

The foamable compositions and foams are suitable for use in treating, ameliorating, reducing or preventing a dermatological, cosmetic or mucosal disorder. More particularly, they are suitable for use where such disorders would otherwise be less responsive when treated with either an anti-infective or a keratolytic agent alone.

In certain aspects, different foamable bases may be used as a vehicle for delivering an effective amount of the combination a keratolytic agent and an augmenting agent. The appropriate foamable base may be selected for compatibility with the selected anti-infective agent or for compatibility with a patient condition, e.g., dry skin.

In certain aspects, a kit provides different platforms as vehicles for delivering an effective amount of an augmenting agent and an anti-infective agent to a target site.

According to one or more embodiments of the present invention, there is provided an oil-in-water emulsion formulation as a suitable vehicle for a keratolytic agent and one or more of an antifungal agent, an antibiotic agent, and an antiviral agent.

According to one or more embodiments of the present invention, there is provided a water-in-oil emulsion formulation as a suitable vehicle for a keratolytic agent and one or more of an antifungal agent, an antibiotic agent, and an antiviral agent.

According to one or more embodiments of the present invention, there is provided waterless oleaginous formulation as a suitable vehicle for a keratolytic agent and one or more of an antifungal agent, an antibiotic agent, and an antiviral agent.

According to one or more embodiments of the present invention, there is provided a waterless polyethylene glycol formulation as a suitable vehicle for a keratolytic agent and one or more of an antifungal agent, an antibiotic agent, and an antiviral agent.

According to one or more embodiments of the present invention, there is provided a waterless propylene glycol formulation as a suitable vehicle for a keratolytic agent and one or more of an antifungal agent, an antibiotic agent, and an antiviral agent.

According to one or more embodiments of the present invention, there is provided a waterless silicone in glycol formulation as a suitable vehicle for a keratolytic agent and one or more of an antifungal agent, an antibiotic agent, and an antiviral agent.

According to one or more embodiments of the present invention, an augmented anti-infective foamable composition includes:

a foamable carrier,

a therapeutically safe and effective concentration of an anti-infective agent,

an augmenting agent selected from the group consisting of a keratolytic agent and a skin penetration enhancer, and

a propellant,

wherein the composition is housed in a container and upon release is expandable to form a breakable foam, and

wherein the foamable carrier is selected to generate a foam of good or excellent quality in the presence of the augmenting agent and anti-infective agent.

In one or more embodiments, propellant comprises a liquefied or compressed gas propellant at a concentration of about 3% to about 25% by weight of the total composition.

In one or more embodiments, the foamable carrier is selected from the group consisting of oil-in-water emulsions, water-in-oil emulsions, waterless oleaginous formulations, waterless polyethylene glycol and propylene glycol based compositions, waterless silicone in polyethylene glycol based compositions and waterless silicone in propylene glycol based compositions.

In one or more embodiments, the foamable carrier comprises a solvent, a surface active agent, and one or more optional agents selected from the group consisting of a one or more optional agents selected from the group comprising a surfactant, co-emulsifier or foam stabilizer; a polymeric agent, a viscosity, bulking or firming agent; a foam adjuvant; a co-solvent; a penetration enhancer, a stabilizer, a modulating agent; a drying agent, and an agent capable of having an occlusive effect.

In one or more embodiments, the foamable carrier comprises an oil-in-water emulsion comprising:
a hydrophobic solvent, at a concentration of about 2% to about 75% by weight;

about 0.1% to about 5% by weight of a surface-active agent; and

optionally about 0.01% to about 5% by weight of at least one polymeric agent; and water.

In one or more embodiments, the foamable carrier comprises a water-in-oil emulsion comprising:

a hydrophobic solvent, at a concentration of about 70% to about 96.5% by weight;

about 0.1% to about 5% by weight of a surface-active agent;

optionally about 0.01% to about 5% by weight of at least one polymeric agent, and water.

In one or more embodiments, the foamable carrier comprises a waterless oleaginous composition comprising:

a hydrophobic solvent at a concentration of about 70% to about 96.5% by weight, or up to 99% by weight;

about 0.1% to about 5% by weight of a surface-active agent; and

optionally about 0.01% to about 5% by weight of at least one polymeric agent.

In one or more embodiments, the foamable carrier comprises a waterless polyethylene glycol and propylene glycol based compositions comprising:

a solvent comprising a polyethylene glycol, a propylene glycol, or mixtures thereof present at a concentration of about 70% to about 96.5% by weight, or up to 99% by weight, of the total composition;

about 0.1% to about 5% by weight of a surface-active agent;

optionally about 0.01% to about 5% by weight of at least one polymeric agent.

In one or more embodiments, the foamable carrier comprises a waterless silicone in propylene glycol or polyethylene glycol based compositions comprising:

a silicone;

a propylene glycol or polyethylene glycol, or mixtures thereof wherein the glycol is present at a concentration of about 70% to about 96.5% by weight, or up to 99% by weight, of the total composition;

optionally at least one polymeric agent at a concentration of about 0.1% to about 5% by weight of the total composition, wherein the at least one polymeric agent is selected from a bioadhesive agent, a gelling agent, a film forming agent and a phase change agent; and

at least one surface-active agent at a concentration of about 0.1% to less than about 10% by weight of the total composition.

According to one or more embodiments of the present invention, a foamable augmented anti-infective composition comprises a solvent, a surfactant, an augmenting agent (consisting of one or more of a keratolytic agent and a penetrating enhancer having keratolytic effects), one or more anti-infective agents (selected from the group consisting of antibiotic agents, antiviral agents and antifungal agents), a propellant and one or more optional agents selected from the group comprising a surfactant, co-emulsifier or foam stabilizer, a polymeric agent, a viscosity, bulking or firming agent, a foam adjuvant, a co-solvent, a modulating agent, a penetration enhancer and an agent capable of having an occlusive effect. The composition is stored in an aerosol container and upon release expands to form a breakable foam. The presence of an augmenting agent and an anti-infective agent does not prevent a foam of good or satisfactory quality from being produced. The foam base, including optional ingredients are selected to provide a foam of ‘good’ or ‘satisfactory’ quality.

In one or more embodiments of the present invention there is also provided an augmented anti-infective composition wherein one of the one or more optional agents is a penetration enhancer that does not have a keratolytic like effect. Further or alternatively the penetration enhancer can be a combination of penetration enhancers. Where the augmentation agent comprises at least a keratolytic agent, then the optional penetration enhancer may, but need not, have a keratolytic effect.

### DETAILED DESCRIPTION OF THE INVENTION

In one or more embodiments of the present invention, keratolytic agents and penetration enhancers having a keratolytic like effect (hereinafter “augmenting agents”) facilitate the delivery active agents to a target site in a foamable composition and foam.

Foamable compositions and foams comprising a combination of an augmenting agent and one or more anti-infective agents are suitable for use in treating, ameliorating, retarding, reducing or preventing a dermatological, cosmetic or mucosal disorder or infection. More particularly, they are suitable for use where such disorders or infection would otherwise be less responsive when only one of the combination of the agents is present in the foamable composition and foam.

In one or more embodiments, different foamable platforms are used as vehicles for delivering an effective amount of augmenting agent and one or more anti-infective agents to a target site.

In one or more embodiments, there is provided a kit for the delivery of keratolytic agents, active agents and penetration enhancers having a keratolytic like effect to a target site or area in one or more foamable compositions and foams.

The foamable composition may be administered to the skin, a body surface, a body cavity or mucosal surface, e.g., the mucosa of the nose, mouth, eye, ear, respiratory system, vagina or rectum (severally and interchangeably termed herein “target site”) and includes (1) a therapeutically safe and effective concentration of an anti-infection agent comprising one or more of an antifungal, an antibiotic and an antiviral agent, and (2) an augmenting agent comprising a keratolytic agent or a skin penetration enhancer which has a keratolytic like effect or both.

In one or more embodiments of the present invention, a foamable composition may be formulated so that the anti-infection agent is released slowly or after an appropriate time delay to allow for the augmentation agent to have an augmentation effect. An augmentation effect is an effect that potentiates, improves, or increases, the effect of an anti-infective agent. It can also include an action that raises the likelihood of anti-infective agent being more effective. It can work at the level of achieving a similar or improved effect with a lower dosage and/or it can provide the active ingredient better access to a target. Slow release may be accomplished in any of the standard ways known in the art. For example, active ingredient may be embedded in a matrix of slowly dissolving substance, which releases active ingredient as the matrix dissolves; it may be conjugated to and or wrapped in a polymeric substance such that the active ingredient can have its effect once it is exposed; active ingredient may be encapsulated in liposomes; or active ingredient may be provided in a proform which when cleaved becomes active.

Fungi feed off keratin. Thus, keratin layers are a focal center for fungal infections, for example in skin or in a nailbed. Therefore the present invention will initially be exemplified by a combination of a keratolytic agent and an antifungal agent. However the disclosure can likewise apply to bacterial infections and antibiotics or to viral infections and antivirals. In many cases the initial infection may be complicated by a secondary infection, which can be due to a different organism whether of the same class or of a different class such that a site or area may require treatment by two or more different agents in the same composition or foam or in more than one foamable composition or foam. In a similar fashion, the invention is initially described with reference to use of a keratolytic agent; however it is understood that penetration enhancers exhibiting a keratolytic like effect may also be used. In one or more embodiments combinations of penetration enhancers may be used. If a keratolytic agent is present then the penetration enhancer need not have a keratolytic like effect. In so far as it does have a keratolytic like effect it may act in combination with the keratolytic agent and can be synergistic in action.

The combination of an augmentation agent e.g., a keratolytic agent, and an anti-infective agent, e.g., an antifungal agent, and optionally other active agents, provides a safe and effective, synergistic composition especially for the treatment of fungal disorders and infections and associated disorders and infections. The keratolytic agent breaks down the keratin layer of the skin, where the microorganisms feed or reside. As a result of the keratin breakdown, the microorganisms survival is impeded or compromised in the infected area. The keratin layer in infected skin areas is deformed and thus it is more vulnerable to keratolytic disintegration. Thus, the efficacy of the antifungal or other agent is enhanced when such agent can more easily reach its target site of action; and furthermore, since the keratin layer is significantly reduced after treatment, the recurrence of fungi is decreased too. Consequently, an application of the foamable composition according to one or more embodiments described herein is expected to result in an accelerated improvement of the infection and may require a lower frequency of treatment and/or a shorter period of therapy. Likewise, the likelihood of secondary infection may be reduced or alternatively the secondary infection may then become more susceptible to treatment. A foamable pharmaceutical composition is very superior in its usability, when compared with a cream or an ointment. When applied onto the afflicted body surface of mammals, i.e., humans or animals, it is in a foam state, allowing free application without spillage. Upon application of a brief mechanical force, the foam spreads freely spreads onto the surface, and seeps into any fold of the skin; and is rapidly absorbed, whereas creams and ointments require extensive rubbing in order to achieve acceptable absorption. Compliance may also be higher by use of foams particularly where the skin is sore or inflamed since the application of a foam should involve less mechanical force.

Organisms like moist or damp environments. In one or more embodiments there is provided foamable waterless or substantially waterless foamable compositions and foams comprising (1) a therapeutically safe and effective concentration of an anti-infection agent comprising one or more of an antifungal, an antibiotic and an antiviral agent; (2) a keratolytic agent or a skin penetration enhancer which has a keratolytic like effect or both; and a hygroscopic solvent, which can help in drying the area of infection and thereby promote healing.

According to one or more preferred embodiments of the present invention the following non limiting combinations are thought to be particularly useful: urea and terbinafine; urea and miconazole; urea and ciclopiroxolamine; urea and itraconazole; urea and clindamycin; urea and acyclovir; urea and mupirocine; retanoic acid and; retanoic acid and miconazole; retanoic acid and ciclopiroxolamine; retanoic acid and itraconazole; retanoic acid and clindamycin; retinoic acid and acyclovir; retinoic acid and mupirocine; azelaic acid and terbinafine; azelaic acid and miconazole; azelaic acid and ciclopiroxolamine; azelaic acid and itraconazole; azelaic acid and clindamycin; azelaic acid and acyclovir; azelaic acid and mupirocine; salicylic acid and terbinafine; salicylic acid and miconazole; salicylic acid and ciclopiroxolamine; salicylic acid and itraconazole; salicylic acid and clindamycin; salicylic acid and acylclovir; salicylic acid and mupirocine; lactic acid and terbinafine; lactic acid and miconazole; lactic acid and ciclopiroxolamine; lactic acid and itraconazole; lactic acid and clindamycin; lactic acid and acyclovir; lactic acid and mupirocine.

Note many of these combinations are foam disrupting combinations and therefore there is provided in one or more embodiments of the present invention foamable compositions which can produce good or excellent quality foam prior to and after the incorporation of active ingredients and despite the difficulty of the foam disrupting characteristics of the active ingredients. In some embodiments the compositions are sufficiently robust to maintain a good or excellent foam with active ingredient combinations that are less disruptive. However, with certain combinations such as combinations of miconazole nitrate and salicylic acid or miconazole nitrate and terbinafine HCL the quality of resultant foam was reduced. This phenomenon was overcome by introducing these combinations into waterless formulations as can be seen below in the section on waterless examples. It was also overcome by introducing these combinations into oil-in-water foamable compositions comprising PPG stearyl ether and octyl dodecanol and buffered to pH 4.7, as can be seen in Example 8. Another way of handling the problem is to replace the active keratolytic ingredient salicylic acid with, for example, lactic acid or urea. Alternatively the miconazole or the terbinafine may be replaced by a different active antifungal with a lesser foam destabilizing effect.

The above embodiments may be further enhanced by the incorporation in the foamable composition of a penetration enhancer, for example dimethyl isosorbide. A list of useful penetration enhancers is provided below.

The above embodiments may be further enhanced by the incorporation in the foamable composition of an anti-oxidant, for example alpha tocopherol. A list of useful anti-oxidants is provided below.

According to one or more embodiments of the present invention, there is also provided an anti-infection kit comprising at least a first anti-infection composition in an aerosol container accommodating a pressurized product and having an outlet capable of releasing the pressurized product as a foam and at least a second anti-infection composition in an aerosol container accommodating a pressurized product and having an outlet capable of releasing the pressurized product as a foam.

In combination kits containing more than one anti-infective foamable composition, at least one of the anti-infective compositions contains an augmentation agent. In one or more other embodiments, the first composition includes an augmentation agent and in one or more other embodiments, the second composition includes an augmentation agent and in one or more further embodiments the first and second compositions include an augmentation agent.

The first and second compositions are different from one another and include (1) a therapeutically safe and effective concentration of an anti-infection agent comprising one or more of an antifungal, an antibiotic and an antiviral agent; and (2) an augmenting agent comprising a keratolytic agent or a skin penetration enhancer which has a keratolytic like effect or both and which is suitable for the treatment, amelioration, retardation, reduction or prevention of a dermatological, cosmetic or mucosal disorder or infection.

In one or more embodiments, the first and second compositions utilize the same or a closely similar platform but comprise different active and/or augmentation agent combinations.

In one or more embodiments, the first composition is an antifungal composition and the second composition is an antibiotic composition. By “antifungal composition” it is meant a foamable composition including an antifungal agent and an augmenting agent. By “antibiotic composition” it is meant a foamable composition including an antibiotic agent and an augmenting agent.

In one or more embodiments, the first composition is an antifungal composition and the second composition is an antiviral composition. By “antiviral composition” it is meant a foamable composition including an antiviral agent and an augmenting agent.

In one or more embodiments, the first composition is an antibiotic composition and the second composition is an antiviral composition.

In one or more other embodiments, the first composition is an antifungal and antibiotic composition and the second composition is an antiviral composition.

In one or more other embodiments, the first composition is an antifungal and antiviral composition and the second composition is an antibiotic composition.

In one or more other embodiments, the first composition is an antibiotic and antiviral composition and the second composition is an antifungal composition.

In one or more embodiments, the second composition includes a foamable base that is different from the foamable base selected for the first composition. In some embodiments the active and augmentation agent combinations are the same or similar and in other embodiments they are different depending on the condition(s) to be treated or prevented. Exemplary foamable bases include oil-in-water emulsions, water-in-oil emulsions, waterless oleaginous formulations, waterless polyethylene glycol and propylene glycol based compositions, waterless silicone in polyethylene glycol based compositions and waterless silicone in propylene glycol based compositions. Suitable foamable bases may be selected, for example, based upon the solubility and/or stability of the anti-infective agent.

In one or more embodiments, the kit comprises at least three anti-infection compositions, wherein the first composition is antifungal augmentation composition, the second composition is an antibiotic augmentation composition and the third composition is an antiviral augmentation composition. In which case all the platforms (foamable bases) may be the same, closely similar or different.

In one or more embodiments, the first composition comprises at least one augmentation active agent, namely an augmentation agent or an active agent with augmentation qualities. In other words the active agent has inherent keratolytic properties suitable for use in the treatment, amelioration, retardation, reduction or prevention of a dermatological, cosmetic or mucosal disorder or infection and the second composition comprises at least one anti-infective active agent suitable for use in the treatment, amelioration, retardation, reduction or prevention of a dermatological, cosmetic or mucosal disorder or infection.

According to one or more embodiments of the present invention, there is also provided an augmented anti-infection kit comprising a dual aerosol dispenser and a dispenser head for use with the dual aerosol dispenser. The dual aerosol dispenser comprises two aerosol containers, wherein a first composition is contained in a first pressurized container and a second composition is contained in a second pressurized container. Additional aerosol containers may be included for additional compositions.

The dispenser head of the dual or multiple aerosol dispenser comprises an actuator to simultaneously actuate the plurality of containers, and a flow guide. The flow guide comprises a plurality of flow conduits disposed within the flow guide. Each of the plurality of flow conduits includes an inlet through a wall of the flow guide connecting with a flow conduit and an outlet from a flow conduit through a wall of the flow guide.

Each of the plurality of inlets and containers has a linker to link an inlet and a container so as to allow the contents of the container upon actuation to pass through the inlet and through the flow conduit to reach and pass through the outlet.

The flow guide is structured and positioned to allow simultaneous flow communication between each of the plurality of flow conduits and the plurality of outlets are structured and positioned to allow substantially contemporaneously dispensing and/or combining of the content from a plurality of containers at a location external to the dispensing head. Further details of a dual aerosol dispenser for use with a foamable anti-infection augmentation composition is found in co-pending, co-owned U.S. patent application Ser. No. 11/520,473, entitled “Apparatus and Method for Releasing a Measure of Content from a Plurality of Containers,” the contents of which are incorporated in its entirety by reference.

According to one or more embodiments of the present invention, there is also provided an augmented anti-infection kit wherein at least one canister includes a metered dosing means for repeatedly delivering a unified quantified dose of foam.

According to one or more embodiments of the present invention, there is also provided an augmented anti-infection kit wherein each canister includes a metered dosing means for repeatedly delivering a unified quantified dose of foam. Further details of a metered dosing means suitable for use with foamable anti-infection augmentation composition is found in co-pending, co-owned U.S. patent application Ser. No. 11/406,133, entitled Apparatus and Method for Releasing a Measured Amount of Content from a Container,” the contents of which are incorporated in its entirety by reference.

According to one or more embodiments of the present invention, there is also provided a method of using an augmented anti-infection kit wherein a subject in need applies a first anti-infection composition to a target area and then applies a second anti-infection composition to the target area. In one or more embodiments, the second anti-infection composition is applied to the target area after allowing for the first composition to be substantially absorbed.

In one or more embodiments, the first and second composition foamable platform bases are aqueous and waterless platform compositions, respectively. The aqueous platform is selected from the group consisting of an oil-in-water emulsion, and a water-in-oil emulsion, and the waterless composition is selected from the group consisting of a waterless oleaginous formulation, a waterless polyethylene glycol or propylene glycol based composition, and waterless silicone in propylene glycol or waterless silicone in polyethylene glycol composition.

Suitable foamable platform bases may be chosen based on a patient needs or characteristics of the anti-infective agent. For example, an individual in need may have moist skin and therefore applying a drying or a moisture absorbing agent, an antiseptic or cleansing formulation may be advantageous. It may also possibly be advantageous to apply an aqueous formulation followed by a waterless composition thereafter.

In other examples, the composition may contain two active agents that require different pH environments in order to remain stable. For example, a first agent may be typically stable at acidic pH and a second agent may be typically stable at basic pH. In other cases, the active agent degrades in the presence of water, and therefore, in such cases the present of water in the composition is not desirable and a waterless base may be used.

According to one or more embodiments of the present invention, there is also provided a method of using a anti-infection kit wherein a subject in need applies a first anti-infection composition to a target area and then applies a second anti-infection composition to the target area after allowing for the first composition to be substantially absorbed. The first composition is selected from the group consisting of an oil-in-water emulsion, and a water-in-oil emulsion, said first composition comprising at least one active agent suitable for the treatment, amelioration, retardation, reduction or prevention of a dermatological, cosmetic or mucosal disorder or infection. The second composition is selected from the group consisting of a waterless oleaginous formulation, a waterless polyethylene glycol or a waterless propylene glycol based composition, a waterless silicone in polyethylene glycol and a waterless silicone in propylene glycol based composition, said second composition comprising at least one active agent suitable for the treatment, amelioration, retardation, reduction or prevention of a dermatological, cosmetic or mucosal disorder or infection. The first and second compositions can be applied in any order.

According to one or more embodiments of the present invention the second composition is applied after allowing for the first composition to be absorbed.

According to one or more embodiments of the present invention the anti-infection composition is a combination of active agents. The combination of active agents may be synergistic.

In other embodiments, the first anti-infection composition comprises a first anti-infection augmenting agent and the second anti-infection composition comprises a second anti-infection augmenting agent wherein the first and second anti-infection augmenting agents work in combination. For example, the first anti-infection augmenting agent acts by having a keratolytic effect and the second works by having a penetration enhancing and keratolytic like effect.

According to one or more embodiments, the first anti-infection composition further comprises moisture absorbers (e.g., polymeric agents; hygroscopic solvents), and/or drying agents (e.g., aluminum salts) and the second anti-infection composition further comprises antiseptic and or cleansing composition or vice versa. In this connection, a moisture absorber can comprise for example a hygroscopic solvent or a polymeric agent.

Exemplary drying agents include an aluminum salt selected from the group consisting of aluminum chloride, aluminum chlorohydrate; aluminum chlorohydrex polyethylene glycol aluminum chlorohydrex; complex polyethylene glycol; aluminum chlorohydrex propylene glycol aluminum chlorohydrex; complex propylene glycol; aluminum dichlorohydrate, aluminum dichlorohydrex, polyethylene aluminum dichlorohydrex; glycol complex polyethylene glycol; aluminum dichlorohydrex propylene glycol aluminum dichlorohydrex; complex propylene glycol; aluminum sesquichlorohydrate; aluminum sesquichlorohydrex polyethylene aluminum sesquichlorohydrex; glycol complex polyethylene glycol; aluminum sesquichlorohydrex propylene aluminum sesquichlorohydrex; glycol complex propylene glycol; aluminum sulfate buffered; aluminum zirconium octachlorohydrate; aluminum zirconium octachlorohydrex aluminum zirconium; glycine complex octachlorohydrex gly; aluminum zirconium pentachlorohydrate; aluminum zirconium pentachlorohydrex aluminum zirconium; glycine complex pentachlorohydrex gly; aluminum zirconium tetrachlorohydrate; aluminum zirconium tetrachlorohydrex aluminum zirconium; glycine complex tetrachlorohydrex gly; aluminum zirconium trichlorohydrate; aluminum zirconium trichlorohydrex glycine aluminum zirconium; complex trichlorohydrex gly; aluminum sulfate buffered with sodium aluminum lactate and ASOS.

Examples of antiseptics agents such as cetrimide, paraoxybenzoic ester, sodium benzoate, potassium sorbate, phenoxyethanol, chlorobenzalconium, chlorobenzetonium, and chlorochlorohexizine.

In use, the anti-infection composition or kit is applied daily, and may be applied for at least three days, or for at least a week, or for at least two weeks.

In certain embodiments, the composition is substantially non-aqueous. However, due to the hygroscopic nature of the waterless solvents such water is rapidly absorbed into the composition and may be associated with them. In addition, some small amount of water may be present. Preferably the composition is “substantially non-aqueous” or “substantially waterless.” The term “substantially non-aqueous” or “substantially waterless is intended to indicate that the composition has a free or non-associated water content below about 5%, preferably below about 2%, such as below about 1.5%. In certain other preferred embodiments the composition is non aqueous or waterless. In one or more alternative embodiments a first active agent, which requires a first pH environment is formulated in a first anti-infection augmentation composition and a second active agent, which requires a second pH environment is formulated in a second anti-infection augmentation composition and the first and second compositions are provided in separate aerosol canisters as a kit.

Short chain alcohols, such as ethanol and propanol are known as polar solvents, however, according to one or more embodiments, the composition of the present invention is alcohol-free or substantially alcohol-free, i.e., free of short chain alcohols. Short chain alcohols, having up to 5 carbon atoms in their carbon chain skeleton and one hydroxyl group, such as ethanol, propanol, isopropanol, butanol, iso-butanol, t-butanol and pentanol, are considered less desirable polar solvents due to their skin-irritating effect.

Thus, in certain embodiments, the composition is substantially alcohol-free and includes less than about 5% final concentration of lower alcohols, preferably less than about 2%, more preferably less than about 1%, In a preferred embodiment the aqueous platform composition is alcohol-free. However, in a waterless or substantially waterless embodiment, a short chain alcohol can be included in the composition, and preferably the ratio between the short chain alcohol and the polyol is less than 1:4 by weight.

Additional therapeutic agents may also be added to the augmented anti-infective composition, wherein the therapeutic agent has a different therapeutic effect than the anti-infective agent.

According to one or more embodiments of the present invention, the anti-infection augmented foamable composition includes:

a foamable platform carrier;

at least one anti-infection agent;

an augmenting agent;

optionally, a second active agent which can provide support to the anti-infection augmentation effect; and

a propellant at a concentration of about 3% to about 45% by weight of the total composition, wherein the composition is stored in an aerosol container and upon release expands to form a foam.

In accordance with one or more embodiments of the present invention, the foamable platform composition comprises a surface active agent and further comprises at least one component selected from the group consisting of a surfactant, co-emulsifier or foam stabilizer, a polymeric gelling agent, a viscosity, bulking or firming agent, a foam adjuvant, a co-solvent, an agent capable of having a drying effect, a penetration enhancer that need not have but may have a keratolytic like effect and a modulating agent.

In accordance with one or more embodiments, a foamable platform aqueous emulsion carrier composition comprises:

an aqueous carrier;

an emollient;

a surfactant;

an optional polymeric agent;

a propellant at a concentration of about 3% to about 45% by weight of the total composition; and

optionally one or more agents selected from the group consisting of a co-emulsifier and foam stabilizer; a viscosity, bulking or firming agent; a stabilizer; a co-solvent; a penetration enhancer; a foam adjuvant; a modulating agent and or an agent capable of having a drying effect,

wherein the presence of an anti-infection and augmenting agent combination and optionally an aluminum salt (a drying agent) does not prevent a foam of ‘good’ or ‘satisfactory’ quality from being produced, and

wherein the composition upon release expands to form a breakable foam.

In accordance with one or more embodiments, a foamable platform waterless carrier composition comprises:

a non-aqueous carrier;

a surfactant;

an optional polymeric agent;

a propellant at a concentration of about 3% to about 45% by weight of the total composition; and

optionally, one or more agents selected from the group consisting of a co-emulsifier, foam stabilizer; a viscosity, bulking or firming agent, a modulating agent and or an agent capable of having a drying effect,

wherein the presence of significant amounts of an anti-infection and augmenting agent combination and optionally an aluminum salt (drying agent) does not prevent a foam of ‘good’ or ‘satisfactory’ quality from being produced, and

wherein the composition upon release expands to form a breakable foam.

The anti-infection topical composition produces a foam having a density of about 0.01 to about 0.2 g/ml. The resultant foam is a breakable foam, which if not subjected to mechanical shear break, is capable of remaining substantially intact without substantial foam collapse for about 60 seconds, or about 120 seconds, or about 300 seconds or more.

In accordance with one or more further embodiments, the propellant is at a concentration from about 3% to about 25% by weight of the total composition.

In accordance with one or more embodiments, the foamable composition is flowable or substantially flowable.

In accordance with one or more embodiments, the flowable carrier composition comprises at least one carrier selected from the group consisting of water, an oil, a silicone oil, an alcohol, a polyol, a polyethylene glycol (PEG), a propylene glycol, and a solvent or combinations thereof.

In accordance with one or more embodiments, the flowable composition comprises at least one aqueous carrier.

In accordance with one or more embodiments, the flowable composition comprises at least one non-aqueous carrier.

In certain embodiments, the carrier further contains a polar solvent.

In accordance with one or more embodiments, the main carrier solvent is at a concentration of about 40% to about 90% by weight of the total composition.

In accordance with one or more embodiments, the surface active agent is a stabilizing combination of at least two surface active agents.

In accordance with one or more embodiments, the surface active agent is at a concentration of about 0.1% to about 15% by weight of the total composition.

In accordance with one or more embodiments, the polymeric agent is at a concentration of about 0.05% to about 5% by weight of the total composition.

The foamable composition can be an emulsion, or microemulsion, including an aqueous phase and an organic phase. In an exemplary embodiment, the foamable anti-infection topical composition is an emulsion comprising water, a hydrophobic solvent, a surface-active agent and a polymeric agent.

In certain embodiments the foamable composition contains up to about 85% water, while in additional embodiments the foamable composition contains up to 50% water.

The augmented anti-infective foamable composition may be an emulsion or microemulsion including a therapeutically safe and effective concentration of an anti-infective agent, an augmenting agent selected from the group consisting of a keratolytic agent and a skin penetration enhancer, a hydrophobic solvent at a concentration of about 2% to about 75% by weight, about 0.1% to about 5% by weight of a surface-active agent, about 0.01% to about 5% by weight of at least one polymeric agent; and a liquefied or compressed gas propellant at a concentration of about 3% to about 25% by weight of the total composition. Water and optional ingredients are added to complete the total mass to 100%. Upon release from an aerosol container, the foamable composition forms an expanded foam suitable for topical administration.

In accordance with one or more embodiments, the composition is an emollient emulsion, that is a composition comprising an emollient and water formulated into an emulsion with the aid of a surfactant. In order to improve the quality and feel of the emollient emulsions, a polymeric agent may be added. It can be an aid, for example, to achieve fine bubble structure and to improve foam stability. The polymeric agent can act to improve the rheology of the composition. The polymeric agent may also for example provide some adhesiveness so that the foam when placed for example on the hand will remain relatively stable for a sufficient period of time in order to allow for its application to a target or area including the skin or a body cavity so that it does not leak or drip at least to any substantial extent. It may also allow for the foam to be handled upside down or vertically without it falling immediately to the floor. The polymeric agent can, for example, be a combination of hydroxy propylmethyl cellulose and xantham gum. In certain other embodiments the polymeric agent is sodium carboxymethyl-cellulose, hydroxyethyl-cellulose, microcrystalline-cellulose, aluminum starch octyl succinate, hydroxypropyl-cellulose such as Klucel EF, and polyacrylates such as carbopol. Other polymeric agents may be used as will be appreciated by a man of the art. A more extensive list of polymeric agents that may be used is provided below. The polymeric agent may fulfill a similar role in a waterless environment.

In accordance with one or more embodiments of the present invention the co-emulsifier is at a concentration of about 0.05% to about 10% by weight of the total composition. The co-emulsifier of the present invention is a substance with surfactant or emulsifier like properties that supports or assists the action of the main surfactant or emulsifier of the composition.

In accordance with one or more embodiments of the present invention the viscosity, bulking or firming agent is at a concentration of about 0.1% to about 15% by weight of the total composition. These terms are understood in the art and In very general terms indicate substances that are suitable for increasing the viscosity, the volume and the hardness of a foam composition

In accordance with one or more embodiments of the present invention the stabilizer is at a concentration of about 0.1% to about 10% by weight of the total composition. A stabilizer in general terms is a substance which acts to stabilize the foamable composition such that for example an emulsion has an improved stability and or which acts to stabilize the resultant foam so that the foam has an improved stability.

In accordance with one or more embodiments of the present invention the co-solvent is at a concentration of about 0.1% to about 48% by weight of the total composition, preferably about 0.1% to about 30% by weight of the total composition. A co-solvent is a second or further solvent and can have any number of uses. In some embodiments it can help to improve the formulation and or foam. In other embodiments it can increase the solubility of an active ingredient. In still further embodiments it can help to reduce the amount of another solvent and therefore render the composition less or non-irritating.

In accordance with one or more embodiments of the present invention the penetration enhancer is at a concentration of about 0.1% to about 30% by weight of the total composition.

In accordance with one or more embodiments of the present invention the agent capable of having a drying effect is at a concentration of about 0.5% to about 20% by weight of the total composition.

Optionally, in one or more embodiments the emulsion-type foamable composition further contains a foam adjuvant agent, selected from the group consisting of a fatty alcohol having 15 or more carbons in their carbon chain; a fatty acid having 16 or more carbons in their carbon chain.

In certain embodiments the hydrophobic carrier is an oil. Exemplary oils include mineral oil, silicone oil, a triglyceride and an ester of a fatty acid.

According to one or more embodiments, the foamable composition is substantially alcohol-free, i.e., free of short chain alcohols. Short chain alcohols, having up to 5 carbon atoms in their carbon chain skeleton and one hydroxyl group, such as ethanol, propanol, isopropanol, butanol, iso-butanol, t-butanol and pentanol, are considered less desirable solvents or polar solvents due to their skin-irritating effect. Thus, the composition is substantially alcohol-free and includes less than about 5% final concentration of lower alcohols, preferably less than about 2%, more preferably less than about 1%.

In an exemplary embodiment, the foamable anti-infection topical composition is an oleaginous foamable composition including at least one solvent selected from a hydrophobic solvent, a silicone oil, an emollient, a polar solvent and mixtures thereof, wherein the solvent is present at a concentration of about 70% to about 96.5% by weight of the total composition; at least a non-ionic surface-active agent; and at least one polymeric agent.

In an exemplary embodiment, the foamable anti-infection topical composition includes more than 50% of a polar, a surface-active agent and a polymeric agent. Solvent. “Polar solvent,” as used herein, means a material that produces a uniform, clear or hazy, mixture when combined with at least a weight equivalent of water.

In accordance with one or more embodiments, a method of treating, alleviating or preventing a dermatological reaction, sensation or disorder of a mammalian subject includes:

administering an effective amount of an anti-infection foamable emollient emulsion carrier composition to a target site on a mammalian subject, and

applying mechanical shear break to the applied foam such that it is spread at, about and within the target site.

The anti-infection foamable emollient emulsion composition includes:

an aqueous carrier at a concentration of about 40% to about 90% by weight of the total composition,

an emollient at a concentration of about 5% to about 15% by weight of the total composition,

a surfactant at a concentration of about 0.1% to about 10% by weight of the total composition,

a polymeric agent at a concentration of about 0.1% to about 5% by weight of the total composition, a propellant at a concentration of about 3% to about 25% by weight of the total composition,

at least one active agent in an effective amount which is intended to prevent, alleviate or treat a dermatological disorder;

an augmenting agent selected from the group consisting of a keratolytic agent and a penetrating agent, and

optionally a co-emulsifier and foam stabilizer at a concentration of about 0.1% to about 5% by weight of the total composition; a viscosity, bulking or firming agent at a concentration of about 0.1% to about 15% by weight of the total composition; a stabilizer; a co-solvent at a concentration of about 0.1% to about 20% by weight of the total composition; a penetration enhancer at a concentration of about 0.1% to about 20% by weight of the total composition; a modulating agent and or an agent capable of having a drying effect at a concentration of about 5% to about 30% by weight of the total composition; and the polymeric agent is selected from the group consisting of a bioadhesive agent, a gelling agent, a film forming agent and a phase change agent,

wherein the presence of significant amounts of anti-infection and augmenting agents in combination and optionally an aluminum salt (as a drying agent) does not prevent a foam of ‘good’ or ‘satisfactory’ quality from being produced; and

wherein the composition is stored in an aerosol container and upon release expands to form a breakable foam.

In accordance with one or more embodiments, there is provided a method of treating, alleviating or preventing a dermatological reaction, sensation or disorder of a mammalian subject, comprising:

administering an effective amount of a anti-infection substantially waterless or waterless foamable composition to a target site on a mammalian subject, and

applying mechanical shear break to the applied foam such that it is spread at, about and within the target site.

The anti-infection substantially waterless or waterless foamable composition includes:

i. a non-aqueous carrier at a concentration of about 40% to about 90% by weight of the total composition;

ii. a surfactant at a concentration of about 0.1% to about 10% by weight of the total composition; and or a polymeric agent at a concentration of about 0.1% to about 5% by weight of the total composition;

iii. at least one active agent in an effective amount, which is intended to prevent, alleviate, treat a dermatological disorder;

an augmenting agent selected from the group consisting of a keratolytic agent and a penetrating agent, and

iv. a propellant at a concentration of about 3% to about 25% by weight of the total composition

and optionally, a silicone, a co-emulsifier and foam stabilizer at a concentration of about 0.1% to about 5% by weight of the total composition; and a viscosity, bulking or firming agent at a concentration of about 0.1% to about 15% by weight of the total composition,

wherein the polymeric agent is selected from the group consisting of a bioadhesive agent, a gelling agent, a film forming agent and a phase change agent;

wherein the presence of significant amounts of an anti-infection augmentation combination and optionally an aluminum salt in a composition does not prevent a foam of ‘good’ or ‘satisfactory’ quality from being produced; and

wherein the composition is stored in an aerosol container and upon release expands to form a breakable foam.

Any of the above foamable composition further includes about 0.1% to about 5% by weight of a foam adjuvant is selected from the group consisting of a fatty alcohol having 15 or more carbons in their carbon chain; a fatty acid having 16 or more carbons in their carbon chain; a fatty alcohol, derived from beeswax and including a mixture of alcohols, a majority of which has at least 20 carbon atoms in their carbon chain; a fatty alcohol having at least one double bond; a fatty acid having at least one double bond; a branched fatty alcohol; a branched fatty acid; a fatty acid substituted with a hydroxyl group; cetyl alcohol; stearyl alcohol; arachidyl alcohol; behenyl alcohol; 1-triacontanol; hexadecanoic acid; stearic acid; arachidic acid; behenic acid; octacosanoic acid; 12-hydroxy stearic acid and any mixture thereof.

In one or more embodiments, the carrier or composition comprises a single phase, and in some embodiments, is a non-aqueous single phase, such as a waterless polyethylene glycol based formulation or a waterless propylene glycol based formulation.

In one or more embodiments, the carrier or composition comprises an emulsion or a microemulsion, such as a water-in-oil emulsion.

For the non-waterless carriers and compositions, optional ingredients are added to complete the total mass to 100%. For the waterless carriers and compositions, a non-aqueous solvent and optional ingredients are added to complete the total mass of 100%. In certain embodiments the propellant is added to the total mass. Upon release from an aerosol container, the foamable composition forms expanded foam suitable for topical administration. The expanded mass will correspond to or reflect the formulation prior to the addition of propellant.

The anti-infective agent can be one or more of an antifungal agent, one or more of an antibiotic agent or one or more of an antiviral agent or combinations thereof. In general terms any anti-infective, which exhibits a relatively strong acidic or basic nature could potentially display anti keratolytic like activity. In certain embodiments the anti-infective agent can have keratolytic like properties and in other certain other embodiments a keratolytic agent may have anti-infective like qualities. For example, azelaic acid, salicylic acid and some dicarboxylic acids, when applied topically or to a body cavity may kill bacteria.

The Antifungal Agent

The active agent according may be an antifungal agent, also termed “animistic.” The terms “antifungal” and “animistic” as used herein include, but is not limited to, any substance being destructive to or inhibiting the growth of fungi and yeast or any substance having the capacity to inhibit the growth of or to destroy fungi and/or yeast.

In one or more embodiments, the antifungal agent is an agent that is useful in the treatment of a superficial fungal infection of the skin, dermatophytosis, microsporum, trichophyton and epidermophyton infections, candidiasis, oral candidiasis (thrush), candidiasis of the skin and genital mucous membrane, candida paronychia, which inflicts the nail and nail bed and genital and vaginal candida, which inflict genitalia and the vagina. Thus, in one or more embodiments, the antifungal agent is selected from the group including but not limited to, azoles, diazoles, triazoles, miconazole, fluconazole, ketoconazole, clotrimazole, itraconazole, Climbazole, griseofulvin, ciclopirox, ciclopiroxolamine, amorolfine, terbinafine, Amphotericin B, potassium iodide and flucytosine (5FC) at a therapeutically effective concentration.

Azoles are pharmaceutically active compounds that are unsaturated five member ring heterocyclic compound, wherein one, two or three members of the ring are nitrogen atoms, as exemplified in a non-limiting way and illustrated in the following schemes:

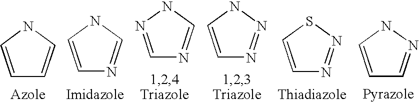

The azole is a compound including an unsaturated five member ring heterocyclic compound, wherein one, two or three members of the ring are nitrogen atoms.

Examples of therapeutic azoles include, but are not limited to, azanidazole, bifonazole, butoconazol, chlormidazole, climbazole, cloconazole, clotrimazole, dimetridazole, econazole, enilconazole, fenticonazole, fezatione, fluconazole, flutrimazole, isoconazole, itraconazole, ketoconazole, lanoconazole, metronidazole, metronidazole benzoate, miconazole, neticonazole, nimorazole, niridazole, omoconazol, ornidazole, oxiconazole, posaconazole, propenidazole, ravuconazole, secnidazol, sertaconazole, sulconazole, thiabendazole, tinidazole, tioconazole, voriconazol and salts and derivatives thereof. Such azoles are mainly used as antifungal agents, yet several of them also possess other therapeutic benefits, such as anti-inflammatory, antibacterial and antiviral effects. Additional non-limiting exemplary classes of azoles include oxazoles, thiazoles, thiadiazoles and thiatriazoles, benzimidazoles, and salts and derivatives thereof.

In an embodiment, the azole is metronidazole.

In one or more embodiments, the antifungal agent is a peptide that possesses an antibacterial and/or an antifungal activity. Such peptide can be naturally occurring and can be obtained from a herbal or a vertebrate source.

In an embodiment of the present invention, the antifungal agent is a polyene. Polyene compounds are so named because of the alternating conjugated double bonds that constitute a part of their macrolide ring structure. Polyenes include, but are not limited to, amphotericin, aureofungin, ayfactin, azalomycin, blasticidin, candicidin, candicidin methyl ester, candimycin, candimycin methyl ester, chinopricin, filipin, flavofungin, fradicin, hamycin, hydropricin, levorin, lucensomycin, lucknomycin, mediocidin, mediocidin methyl ester, mepartricin, methylamphotericin, natamycin, niphimycin, nystatin, oxypricin, partricin, pentamycin, perimycin, pimaricin, primycin, proticin, rimocidin, sistomycosin, sorangicin, trichomycin and analogs, salts and derivatives thereof.

In an embodiment, the antifungal agent is a pyrimidine, such as Flucytosine.

In an embodiment, the antifungal agent is an allylamine, such as terbinafine and naftifine.

In an embodiment, the antifungal agent is a morpholine derivative, such as amorolfine.

In an embodiment, the antifungal agent is selected from the group consisting of ciclopirox, ciclopiroxolmine, griseofulvin,

In an embodiment, the antifungal agent is a Thiocarbamate, such as tolnaftate.

In an embodiment, the antifungal agent is a Sulfonamide, such as mafenide and dapsone.

In an embodiment, the antifungal agent consists of plant oil or a plant extract possessing antifungal activity; or a plant oil or extract which contains antifungal agents. Non-limiting examples of plants containing agents include, but are not limited to, anise, basil, bergemont, burdock, buchu, chaparral, camphor, cardamom, carrot, canola, cassia, catnip, cedarwood, citronella, clove, couchgrass, cypress, echinacea, eucalyptus, faenia interjecta, garlic, ginger, grapefruit, holy thistle, hops, hyssop, jasmine, jojova, lavender, lavandin, lemon, lime, mandarin, marigold, marjoram, maytenus ilicifolia, maytenus evonymoides, maytenus aquifolia, micromonospora, myrrh, neroli, nutmeg, orange, ordyceps sinensis, peppermint, perilla, petitgrain, plantain, putterlickia verrucosa, putterlickia pyracantha, putterlickia retrospinosa, rosemary, sage, spearmint, star anise, St. John's wort, red clover, tangerine, tea tree, terfezia clayeryi, thyme vanilla, verbena, white clover and yellow dock.

In an embodiment, the antifungal agent is an anti-microbial metal. A number of metals ions been shown to possess antibiotic activity, including silver, copper, zinc, mercury, tin, lead, bismutin, cadmium, chromium and ions thereof. It has been theorized that these anti-microbial metal ions exert their effects by disrupting respiration and electron transport systems upon absorption into bacterial or fungal cells. Anti-microbial metal ions of silver, copper, zinc, and gold, in particular, are considered safe for in vivo use. Anti-microbial silver and silver ions are particularly useful due to the fact that they are not substantially absorbed into the body.

Thus, in one or more embodiment, the anti-microbial metal consists of an elemental metal, selected from the group consisting of silver, copper, zinc, mercury, tin, lead, bismutin, cadmium, chromium and gold, which is suspended in the composition as particles, microparticles, nanoparticles or colloidal particles. The anti-microbial metal can further be intercalated in a chelating substrate.

In further embodiments, the anti-microbial metal is ionic. The ionic antibiotic metal can be presented as an inorganic or organic salt (coupled with a counterion), an organometallic complex or an intercalate. Non-limiting examples of counter inorganic and organic ions are sulfadiazine, acetate, benzoate, carbonate, iodate, iodide, lactate, laurate, nitrate, oxide, palmitate, a negatively charged protein. In preferred embodiments, the antibiotic metal salt is a silver salt, such as silver acetate, silver benzoate, silver carbonate, silver iodate, silver iodide, silver lactate, silver laurate, silver nitrate, silver oxide, silver palmitate, silver protein, and silver sulfadiazine.

Yet, in another embodiment, the antifungal agent is an oxidizing agent or a substance that releases free radicals and/or active oxygen. Exemplary oxidizing agents are hydrogen peroxide, benzoyl peroxide, elemental halogen species (compounds), as well as oxygenated halogen species (compounds), bleaching agents (e.g., sodium, calcium or magnesium hypochloride and the like), perchlorite species (compounds), iodine and iodate compounds. Organic oxidizing agents are also included in the definition of “oxidizing agent” according to the present invention, such as quinones. Such agents possess a potent broad spectrum activity.

In one or more embodiments, the antifungal agent is an agent that is useful in the treatment of a superficial fungal infection of the skin, dermatophytosis, microsporum, trichophyton and epidermophyton infections, candidiasis, oral candidiasis (thrush), candidiasis of the skin and genital mucous membrane, candida paronychia, which inflicts the nail and nail bed and genital and vaginal candida, which inflict genitalia and the vagina.

Suitable antimycotics include but are not limited to allylamines, amorolfine, amphotericin B, azole compounds, bifonazole, butoconazole, chloroxine, clotrimazole, ciclopirox olamine, clotrimazole, econazole, elubiol, fenticonazole, fluconazole, flucytosine (5FC), griseofulvin, itraconazole, ketoconazole, mafenide acetate, miconazole, naftifine, natamycin, tolnaftate, nystatin, polyenes, oxiconazole, sulbentine, sulconazole, terbinafine, terconazole, tioconazole, undecylenic acid and derivatives, esters, salts and mixtures thereof.

In one or more embodiments, the antifungal agent is a miconazole such as miconazole nitrate.

In further embodiments the agent is a cationic antimicrobial agent. The outermost surface of bacterial and fungal cells universally carries a net negative charge, making them sensitive to cationic substances. Examples of cationic antibiotic agents include: quaternary ammonium compounds, such as alkyltrimethyl ammonium bromides, benzalkonium chloride, dialkylbenzyl ammonium halides, and dimmers thereof, which bear bi-polar positive charges in conjunction with interstitial hydrophobic regions.

The term “safe and effective amount” as used herein, means an amount of an active ingredient high enough to modify the wound condition to be treated or to deliver the desired skin benefit, but low enough to avoid serious side effects, at a reasonable benefit to risk ratio within the scope of sound medical judgment. What is a safe and effective amount of the active ingredient will vary with the specific active, the ability of the active to penetrate through the skin, the age, health condition, and skin condition of the user, and other like factors.

By “pharmaceutically-acceptable salts” are meant any of the commonly-used salts that are suitable for use in contact with the tissues of humans without undue toxicity, irritation, incompatibility, instability, irritation, allergic response, and the like.

Dandruff can be associated with fungal infections. Thus in an embodiment of the present invention, the therapeutic agent is an antidandruff agent. Suitable antidandruff agents include but are not limited to aminexil, benzalkonium chloride, benzethonium chloride, 3-bromo-1-chloro-5,5-dimethyl-hydantoin, chloramine B, chloramine T, chlorhexidine, N-chlorosuccinimide, climbazole, 1,3-dibromo-5,5-dimethylhydantoin, 1,3-dichloro-5,5-dimethyl-hydantoin, betulinic acid, betulonic acid, celastrol, crataegolic acid, cromakalin, cyproterone acetate, dutasteride, finesteride, ibuprofen, ketoconozole, oleanolic acid, phenyloin, picrotone olamine, salicylic acid, selenium sulphides, triclosan, triiodothyronine, ursolic acid, zinc gluconate, zinc omadine, zinc pyrithione and derivatives, esters, salts and mixtures thereof.

Antibiotics

In an embodiment of the present invention, the therapeutic agent is an antibiotic. The terms “antibiotic” as used herein shall include, but is not limited to, any substance being destructive to or inhibiting the growth of bacteria or any substance having the capacity to inhibit the growth of or to destroy bacteria.

In one or more embodiments, the antibiotic agent is selected from the group consisting of a beta-lactam antibiotic, an aminoglycoside, an ansa-type antibiotic, an anthraquinone, an azole, an antibiotic glycopeptide, a macrolide, an antibiotic nucleoside, an antibiotic peptide, an antibiotic polyene, an antibiotic polyether, an antibiotic quinolone, an antibiotic steroid, a sulfonamide, an antibiotic metal, an oxidizing agent, a periodate, a hypochlorite, a permanganate, a substance that release free radicals and/or active oxygen, a cationic antimicrobial agent, a quaternary ammonium compound, a biguanide, a triguanide, a bisbiguanide, a polymeric biguanide, and analogs, derivatives, salts, ions and complexes thereof.

Suitable antibiotics include but are not limited to amanfadine hydrochloride, amanfadine sulfate, amikacin, arnikacin sulfate, aminoglycosides, amoxicillin, ampicillin, ansamycins, bacitracin, beta-lactams, candicidin, capreomycin, carbenicillin, cephalexin, cephaloridine, cephalothin, cefazolin, cephapirin, cephradine, cephaloglycin, chloramphenicols, chlorhexidine, chlorhexidine gluconate, chlorhexidine hydrochloride, chloroxine, chlorquinaldol, chlortetracycline, chlortetracycline hydrochloride, ciprofloxacin, circulin, clindamycin, clindamycin hydrochloride, clotrimazole, cloxacillin, demeclocycline, diclosxacillin, diiodohydroxyquin, doxycycline, ethambutol, ethambutol hydrochloride, erythromycin, erythromycin estolate, erythromycin stearate, farnesol, floxacillin, gentamicin, gentamicin sulfate, gramicidin, griseofulvin, haloprogin, haloquinol, hexachlorophene, iminocyldline, iodate, iodine, iodochlorhydroxyquin, kanamycin, kanamycin sulfate, lincomycin, lineomycin, lineomycin hydrochloride, macrolides, meclocycline, methacycline, methacycline hydrochloride, methenamine, methenamine hippurate, methenamine mandelate, methicillin, metronidazole, miconazole, miconazole hydrochloride, microcrystalline and nanocrystalline particles of silver, copper, zinc, mercury, tin, lead, bismuth, cadmium and chromium, minocycline, minocycline hydrochloride, mupirocin, nafcillin, neomycin, neomycin sulfate, netilmicin, netilmicin sulfate, nitrofurazone, norfloxacin, nystatin, octopirox, oleandomycin, orcephalosporins, oxacillin, oxytetracycline, oxytetracycline hydrochloride, parachlorometa xylenol, paromomycin, paromomycin sulfate, penicillins, penicillin G, penicillin V, pentamidine, pentamidine hydrochloride, phenethicillin, polymyxins, quinolones, streptomycin sulfate, tetracycline, tobramycin, tolnaftate, triclosan, trifampin, rifamycin, rolitetracycline, spectinomycin, spiramycin, streptomycin, sulfonamide, tetracyclines, tetracycline, tobramycin, tobramycin sulfate, triclocarbon, triclosan, trimethoprim-sulfamethoxazole, tylosin, vancomycin, eyrothricin and derivatives, esters, salts and mixtures thereof.

Beta-lactam antibiotics include, but are not limited to, 2-(3-alanyl)clavam, 2-hydroxymethylclavam, 8-epi-thienamycin, acetyl-thienamycin, amoxicillin, amoxicillin sodium, amoxicillin trihydrate, amoxicillin-potassium clavulanate combination, ampicillin, ampicillin sodium, ampicillin trihydrate, ampicillin-sulbactam, apalcillin, aspoxicillin, azidocillin, azlocillin, aztreonam, bacampicillin, biapenem, carbenicillin, carbenicillin disodium, carfecillin, carindacillin, carpetimycin, cefacetril, cefaclor, cefadroxil, cefalexin, cefaloridine, cefalotin, cefamandole, cefamandole, cefapirin, cefatrizine, cefatrizine propylene glycol, cefazedone, cefazolin, cefbuperazone, cefcapene, cefcapene pivoxil hydrochloride, cefdinir, cefditoren, cefditoren pivoxil, cefepime, cefetamet, cefetamet pivoxil, cefixime, cefmenoxime, cefmetazole, cefminox, cefminox, cefmolexin, cefodizime, cefonicid, cefoperazone, ceforanide, cefoselis, cefotaxime, cefotetan, cefotiam, cefoxitin, cefozopran, cefpiramide, cefpirome, cefpodoxime, cefpodoxime proxetil, cefprozil, cefquinome, cefradine, cefroxadine, cefsulodin, ceftazidime, cefteram, cefteram pivoxil, ceftezole, ceftibuten, ceftizoxime, ceftriaxone, cefuroxime, cefuroxime axetil, cephalosporin, cephamycin, chitinovorin, ciclacillin, clavulanic acid, clometocillin, cloxacillin, cycloserine, deoxy pluracidomycin, dicloxacillin, dihydro pluracidomycin, epicillin, epithienamycin, ertapenem, faropenem, flomoxef, flucloxacillin, hetacillin, imipenem, lenampicillin, loracarbef, mecillinam, meropenem, metampicillin, meticillin, mezlocillin, moxalactam, nafcillin, northienamycin, oxacillin, panipenem, penamecillin, penicillin, phenethicillin, piperacillin, tazobactam, pivampicillin, pivcefalexin, pivmecillinam, pivmecillinam hydrochloride, pluracidomycin, propicillin, sarmoxicillin, sulbactam, sulbenicillin, talampicillin, temocillin, terconazole, thienamycin, ticarcillin and analogs, salts and derivatives thereof.

Aminoglycosides include, but are not limited to, 1,2′-N-DL-isoseryl-3′,4′-dideoxykanamycin B, 1,2′-N-DL-isoseryl-kanamycin B, 1,2′-N—-[(S)-4-amino-2-hydroxybutyryl]-3′,4′-dideoxykanamycin B, 1,2′-N—[(S)-4-amino-2-hydroxybutyryl]-kanamycin B, 1-N-(2-Aminobutanesulfonyl)kanamycin A, 1-N-(2-aminoethanesulfonyl)3′,4′-dideoxyribostamycin, 1-N-(2-Aminoethanesulfonyl)3′-deoxyribostamycin, 1-N-(2-aminoethanesulfonyl)3′4′-dideoxykanamycin B, 1-N-(2-aminoethanesulfonyl)kanamycin A, 1-N-(2-aminoethanesulfonyl)kanamycin B, 1-N-(2-aminoethanesulfonyl)ribostamycin, 1-N-(2-aminopropanesulfonyl)3′-deoxykanamycin B, 1-N-(2-aminopropanesulfonyl)3′4′-dideoxykanamycin B, 1-N-(2-aminopropanesulfonyl)kanamycin A, 1-N-(2-aminopropanesulfonyl)kanamycin B, 1-N-(L-4-amino-2-hydroxy-butyryl)2,′3′-dideoxy-2′-fluorokanamycin A, 1-N-(L-4-amino-2-hydroxy-propionyl)2,′3′-dideoxy-2′-fluorokanamycin A, 1-N-DL-3′,4′-dideoxy-isoserylkanamycin B,1-N-DL-isoserylkanamycin, 1-N-DL-isoserylkanamycin B, 1-N-[L-(−)-(alpha-hydroxy-gamma-aminobutyryl)]-XK-62-2, 2′,3′-dideoxy-2′-fluorokanamycin A,2-hydroxygentamycin A3, 2-hydroxygentamycin B, 2-hydroxygentamycin B1, 2-hydroxygentamycin JI-20A, 2-hydroxygentamycin JI-20B, 3″-N-methyl-4″-C-methyl-3′,4′-dodeoxy kanamycin A, 3″-N-methyl-4″-C-methyl-3′,4′-dodeoxy kanamycin B, 3″-N-methyl-4″-C-methyl-3′,4′-dodeoxy-6′-methyl kanamycin B, 3′,4′-Dideoxy-3′-eno-ribostamycin,3′,4′-dideoxyneamine, 3′,4′-dideoxyribostamycin, 3′-deoxy-6′-N-methyl-kanamycin B,3′-deoxyneamine,3′-deoxyribostamycin, 3′-oxysaccharocin,3,3′-nepotrehalosadiamine, 3-demethoxy-2″-N-formimidoylistamycin B disulfate tetrahydrate, 3-demethoxyistamycin B,3-O-demethyl-2-N-formimidoylistamycin B, 3-O-demethylistamycin B,3-trehalosamine,4″, 6″-dideoxydibekacin, 4-N-glycyl-KA-6606VI, 5″-Amino-3′,4′,5″-trideoxy-butirosin A, 6″-deoxydibekacin,6′-epifortimicin A, 6-deoxy-neomycin (structure 6-deoxy-neomycin B),6-deoxy-neomycin B, 6-deoxy-neomycin C, 6-deoxy-paromomycin, acmimycin, AHB-3′,4′-dideoxyribostamycin,AHB-3′-deoxykanamycin B, AHB-3′-deoxyneamine,AHB-3′-deoxyribostamycin,AHB-4″-6″-dideoxydibekacin, AHB-6″-deoxydibekacin,AHB-dideoxyneamine,AHB-kanamycin B, AHB-methyl-3′-deoxykanamycin B, amikacin, amikacin sulfate, apramycin, arbekacin, astromicin, astromicin sulfate, bekanamycin, bluensomycin, boholmycin, butirosin, butirosin B, catenulin, coumamidine gamma1, coumamidine gamma2,D,L-1-N-(alpha-hydroxy-beta-aminopropionyl)-XK-62-2, dactimicin,de-O-methyl-4-N-glycyl-KA-6606VI, de-O-methyl-KA-6606I, de-O-methyl-KA-7038I, destomycin A, destomycin B, di-N6′,O3-demethylistamycin A, dibekacin, dibekacin sulfate, dihydrostreptomycin, dihydrostreptomycin sulfate, epi-formamidoylglycidylfortimicin B, epihygromycin, formimidoyl-istamycin A, formimidoyl-istamycin B, fortimicin B, fortimicin C, fortimicin D, fortimicin KE, fortimicin KF, fortimicin KG, fortimicin KG1 (stereoisomer KG1/KG2), fortimicin KG2 (stereoisomer KG1/KG2), fortimicin KG3, framycetin, framycetin sulphate, gentamicin, gentamycin sulfate, globeomycin, hybrimycin A1, hybrimycin A2, hybrimycin B1, hybrimycin B2, hybrimycin C1, hybrimycin C2, hydroxystreptomycin, hygromycin, hygromycin B, isepamicin, isepamicin sulfate, istamycin, kanamycin, kanamycin sulphate, kasugamycin, lividomycin, marcomycin, micronomicin, micronomicin sulfate, mutamicin, myomycin, N-demethyl-7-O-demethylcelesticetin, demethylcelesticetin, methanesulfonic acid derivative of istamycin, nebramycin, nebramycin, neomycin, netilmicin, oligostatin, paromomycin, quintomycin, ribostamycin, saccharocin, seldomycin, sisomicin, sorbistin, spectinomycin, streptomycin, tobramycin, trehalosmaine, trestatin, validamycin, verdamycin, xylostasin, zygomycin and analogs, salts and derivatives thereof.

Ansa-type antibiotics include, but are not limited to, 21-hydroxy-25-demethyl-25-methylthioprotostreptovaricin, 3-methylthiorifamycin, ansamitocin, atropisostreptovaricin, awamycin, halomicin, maytansine, naphthomycin, rifabutin, rifamide, rifampicin, rifamycin, rifapentine, rifaximin, rubradirin, streptovaricin, tolypomycin and analogs, salts and derivatives thereof.

Antibiotic anthraquinones include, but are not limited to, auramycin, cinerubin, ditrisarubicin, ditrisarubicin C, figaroic acid fragilomycin, minomycin, rabelomycin, rudolfomycin, sulfurmycin and analogs, salts and derivatives thereof.

Antibiotic azoles include, but are not limited to, azanidazole, bifonazole, butoconazol, chlormidazole, chlormidazole hydrochloride, cloconazole, cloconazole monohydrochloride, clotrimazol, dimetridazole, econazole, econazole nitrate, enilconazole, fenticonazole, fenticonazole nitrate, fezatione, fluconazole, flutrimazole, isoconazole, isoconazole nitrate, itraconazole, ketoconazole, lanoconazole, metronidazole, metronidazole benzoate, miconazole, miconazole nitrate, neticonazole, nimorazole, niridazole, omoconazol, ornidazole, oxiconazole, oxiconazole nitrate, propenidazole, secnidazol, sertaconazole, sertaconazole nitrate, sulconazole, sulconazole nitrate, tinidazole, tioconazole, voriconazol and analogs, salts and derivatives thereof.

Antibiotic glycopeptides include, but are not limited to, acanthomycin, actaplanin, avoparcin, balhimycin, bleomycin B (copper bleomycin), chloroorienticin, chloropolysporin, demethylvancomycin, enduracidin, galacardin, guanidylfungin, hachimycin, demethylvancomycin, N-nonanoyl-teicoplanin, phleomycin, platomycin, ristocetin, staphylocidin, talisomycin, teicoplanin, vancomycin, victomycin, xylocandin, zorbamycin and analogs, salts and derivatives thereof.

Macrolides include, but are not limited to, acetylleucomycin, acetylkitasamycin, angolamycin, azithromycin, bafilomycin, brefeldin, carbomycin, chalcomycin, cirramycin, clarithromycin, concanamycin, deisovaleryl-niddamycin, demycinosyl-mycinamycin, Di-O-methyltiacumicidin, dirithromycin, erythromycin, erythromycin estolate, erythromycin ethyl succinate, erythromycin lactobionate, erythromycin stearate, flurithromycin, focusin, foromacidin, haterumalide, haterumalide, josamycin, josamycin ropionate, juvenimycin, juvenimycin, kitasamycin, ketotiacumicin, lankavacidin, lankavamycin, leucomycin, machecin, maridomycin, megalomicin, methylleucomycin, methymycin, midecamycin, miocamycin, mycaminosyltylactone, mycinomycin, neutramycin, niddamycin, nonactin, oleandomycin, phenylacetyldeltamycin, pamamycin, picromycin, rokitamycin, rosaramicin, roxithromycin, sedecamycin, shincomycin, spiramycin, swalpamycin, tacrolimus, telithromycin, tiacumicin, tilmicosin, treponemycin, troleandomycin, tylosin, venturicidin and analogs, salts and derivatives thereof.

Antibiotic nucleosides include, but are not limited to, amicetin, angustmycin, azathymidine, blasticidin S, epiroprim, flucytosine, gougerotin, mildiomycin, nikkomycin, nucleocidin, oxanosine, oxanosine, puromycin, pyrazomycin, showdomycin, sinefungin, sparsogenin, spicamycin, tunicamycin, uracil polyoxin, vengicide and analogs, salts and derivatives thereof.

Antibiotic peptides include, but are not limited to, actinomycin, aculeacin, alazopeptin, amfomycin, amythiamycin, antifungal from Zalerion arboricola, antrimycin, apid, apidaecin, aspartocin, auromomycin, bacileucin, bacillomycin, bacillopeptin, bacitracin, bagacidin, berninamycin, beta-alanyl-L-tyrosine, bottromycin, capreomycin, caspofungine, cepacidine, cerexin, cilofungin, circulin, colistin, cyclodepsipeptide, cytophagin, dactinomycin, daptomycin, decapeptide, desoxymulundocandin, echanomycin, echinocandin B, echinomycin, ecomycin, enniatin, etamycin, fabatin, ferrimycin, ferrimycin, ficellomycin, fluoronocathiacin, fusaricidin, gardimycin, gatavalin, globopeptin, glyphomycin, gramicidin, herbicolin, iomycin, iturin, iyomycin, izupeptin, janiemycin, janthinocin, jolipeptin, katanosin, killertoxin, lipopeptide antibiotic, lipopeptide from Zalerion sp., lysobactin, lysozyme, macromomycin, magainin, melittin, mersacidin, mikamycin, mureidomycin, mycoplanecin, mycosubtilin, neopeptifluorin, neoviridogrisein, netropsin, nisin, nocathiacin, nocathiacin 6-deoxyglycoside, nosiheptide, octapeptin, pacidamycin, pentadecapeptide, peptifluorin, permetin, phytoactin, phytostreptin, planothiocin, plusbacin, polcillin, polymyxin antibiotic complex, polymyxin B, polymyxin B1, polymyxin F, preneocarzinostatin, quinomycin, quinupristin-dalfopristin, safracin, salmycin, salmycin, salmycin, sandramycin, saramycetin, siomycin, sperabillin, sporamycin, a streptomyces compound, subtilin, teicoplanin aglycone, telomycin, thermothiocin, thiopeptin, thiostrepton, tridecaptin, tsushimycin, tuberactinomycin, tuberactinomycin, tyrothricin, valinomycin, viomycin, virginiamycin, zervacin and analogs, salts and derivatives thereof.

In one or more embodiments, the antibiotic peptide is a naturally-occurring peptide that possesses an antibacterial and/or an antifungal activity. Such peptide can be obtained from a herbal or a vertebrate source.

Polyenes include, but are not limited to, amphotericin, amphotericin, aureofungin, ayfactin, azalomycin, blasticidin, candicidin, candicidin methyl ester, candimycin, candimycin methyl ester, chinopricin, filipin, flavofungin, fradicin, hamycin, hydropricin, levorin, lucensomycin, lucknomycin, mediocidin, mediocidin methyl ester, mepartricin, methylamphotericin, natamycin, niphimycin, nystatin, nystatin methyl ester, oxypricin, partricin, pentamycin, perimycin, pimaricin, primycin, proticin, rimocidin, sistomycosin, sorangicin, trichomycin and analogs, salts and derivatives thereof.

Polyethers include, but are not limited to, 20-deoxy-epi-narasin, 20-deoxysalinomycin, carriomycin, dianemycin, dihydrolonomycin, etheromycin, ionomycin, iso-lasalocid, lasalocid, lenoremycin, lonomycin, lysocellin, monensin, narasin, oxolonomycin, a polycyclic ether antibiotic, salinomycin and analogs, salts and derivatives thereof.

Quinolones include, but are not limited to, an alkyl-methylendioxy-4(1H)-oxocinnoline-3-carboxylic acid, alatrofloxacin, cinoxacin, ciprofloxacin, ciprofloxacin hydrochloride, danofloxacin, dermofongin A, enoxacin, enrofloxacin, fleroxacin, flumequine, gatifloxacin, gemifloxacin, grepafloxacin, levofloxacin, lomefloxacin, lomefloxacin, hydrochloride, miloxacin, moxifloxacin, nadifloxacin, nalidixic acid, nifuroquine, norfloxacin, ofloxacin, orbifloxacin, oxolinic acid, pazufloxacine, pefloxacin, pefloxacin mesylate, pipemidic acid, piromidic acid, premafloxacin, rosoxacin, rufloxacin, sparfloxacin, temafloxacin, tosufloxacin, trovafloxacin and analogs, salts and derivatives thereof.

Antibiotic steroids include, but are not limited to, aminosterol, ascosteroside, cladosporide A, dihydrofusidic acid, dehydro-dihydrofusidic acid, dehydrofusidic acid, fusidic acid, squalamine and analogs, salts and derivatives thereof.

Sulfonamides include, but are not limited to, chloramine, dapsone, mafenide, phthalylsulfathiazole, succinylsulfathiazole, sulfabenzamide, sulfacetamide, sulfachlorpyridazine, sulfadiazine, sulfadiazine silver, sulfadicramide, sulfadimethoxine, sulfadoxine, sulfaguanidine, sulfalene, sulfamazone, sulfamerazine, sulfamethazine, sulfamethizole, sulfamethoxazole, sulfamethoxypyridazine, sulfamonomethoxine, sulfamoxol, sulfanilamide, sulfaperine, sulfaphenazol, sulfapyridine, sulfaquinoxaline, sulfasuccinamide, sulfathiazole, sulfathiourea, sulfatolamide, sulfatriazin, sulfisomidine, sulfisoxazole, sulfisoxazole acetyl, sulfacarbamide and analogs, salts and derivatives thereof.

Tetracyclines include, but are not limited to, dihydrosteffimycin, demethyltetracycline, aclacinomycin, akrobomycin, baumycin, bromotetracycline, cetocyclin, chlortetracycline, clomocycline, daunorubicin, demeclocycline, doxorubicin, doxorubicin hydrochloride, doxycycline, lymecyclin, marcellomycin, meclocycline, meclocycline sulfosalicylate, methacycline, minocycline, minocycline hydrochloride, museftamycin, oxytetracycline, rhodirubin, rolitetracycline, rubomycin, serirubicin, steffimycin, tetracycline and analogs, salts and derivatives thereof.

Dicarboxylic acids, having between about 6 and about 14 carbon atoms in their carbon atom skeleton are particularly useful in the treatment of disorders of the skin and mucosal membranes that involve microbial. Suitable dicarboxylic acid moieties include, but are not limited to, adipic acid, pimelic acid, suberic acid, azelaic acid, sebacic acid, 1,11-undecanedioic acid, 1,12-dodecanedioic acid, 1,13-tridecanedioic acid and 1,14-tetradecanedioic acid. Thus, in one or more embodiments of the present invention, dicarboxylic acids, having between about 6 and about 14 carbon atoms in their carbon atom skeleton, as well as their salts and derivatives (e.g., esters, amides, mercapto-derivatives, anhydraides), are useful immunomodulators in the treatment of disorders of the skin and mucosal membranes that involve inflammation. Azelaic acid and its salts and derivatives are preferred. It has antibacterial effects on both aerobic and anaerobic organisms, particularly propionibacterium acnes and staphylococcus epidermidis, normalizes keratinization, and has a cytotoxic effect on malignant or hyperactive melanocytes. In a preferred embodiment, the dicarboxylic acid is azelaic acid in a concentration greater than 10%. Preferably, the concentration of azelaic acid is between about 10% and about 25%. In such concentrates, azelaic acid is suitable for the treatment of a variety of skin disorders, such as acne, rosacea and hyperpigmentation.

In one or more embodiments, the antibiotic agent is an antibiotic metal. A number of metals ions been shown to possess antibiotic activity, including silver, copper, zinc, mercury, tin, lead, bismutin, cadmium, chromium and ions thereof. It has been theorized that these antibiotic metal ions exert their effects by disrupting respiration and electron transport systems upon absorption into bacterial or fungal cells. Anti-microbial metal ions of silver, copper, zinc, and gold, in particular, are considered safe for in vivo use. Anti-microbial silver and silver ions are particularly useful due to the fact that they are not substantially absorbed into the body.

Thus, in one or more embodiment, the antibiotic metal consists of an elemental metal, selected from the group consisting of silver, copper, zinc, mercury, tin, lead, bismutin, cadmium, chromium and gold, which is suspended in the composition as particles, microparticles, nanoparticles or colloidal particles. The antibiotic metal can further be intercalated in a chelating substrate.

In further embodiments, the antibiotic metal is ionic. The ionic antibiotic metal can be presented as an inorganic or organic salt (coupled with a counterion), an organometallic complex or an intercalate. Non binding examples of counter inorganic and organic ions are sulfadiazine, acetate, benzoate, carbonate, iodate, iodide, lactate, laurate, nitrate, oxide, palmitate, a negatively charged protein. In preferred embodiments, the antibiotic metal salt is a silver salt, such as silver acetate, silver benzoate, silver carbonate, silver iodate, silver iodide, silver lactate, silver laurate, silver nitrate, silver oxide, silver palmitate, silver protein, and silver sulfadiazine.

In one or more embodiments, the antibiotic metal or metal ion is embedded into a substrate, such as a polymer, a mineral (such as zeolite, clay and silica).

Oxidizing agents and substances that release free radicals and/or active oxygen. In one or more embodiments, the antibiotic agent comprises strong oxidants and free radical liberating compounds, such as oxygen, hydrogen peroxide, benzoyl peroxide, elemental halogen species, as well as oxygenated halogen species, bleaching agents (e.g., sodium, calcium or magnesium hypochloride and the like), perchlorite species, iodine, iodate, and benzoyl peroxide. Organic oxidizing agents are also included in the definition of “oxidizing agent” according to the present invention, such as quinones. Such agents possess a potent broad spectrum activity

In one or more embodiments the antibiotic agent is a cationic antimicrobial agent. The outermost surface of bacterial cells universally carries a net negative charge, making them sensitive to cationic substances. Examples of cationic antibiotic agents include: quaternary ammonium compounds (QAC's)—QAC's are surfactants, generally containing one quaternary nitrogen associated with at least one major hydrophobic moiety; alkyltrimethyl ammonium bromides are mixtures of where the alkyl group is between 8 and 18 carbons long, such as cetrimide (tetradecyltrimethylammonium bromide); benzalkonium chloride, which is a mixture of n-alkyldimethylbenzyl ammonium chloride where the alkyl groups (the hydrophobic moiety) can be of variable length; dialkylmethyl ammonium halides; dialkylbenzyl ammonium halides; and QAC dimmers, which bear bi-polar positive charges in conjunction with interstitial hydrophobic regions.

In one or more embodiments, the antibiotic agent is selected from the group of biguanides, triguanides, bisbiguanides and analogs thereof.

Guanides, biguanides, biguanidines and triguanides are unsaturated nitrogen containing molecules that readily obtain one or more positive charges, which make them effective antimicrobial agents. The basic structures a guanide, a biguanide, a biguanidine and a triguanide are provided below.

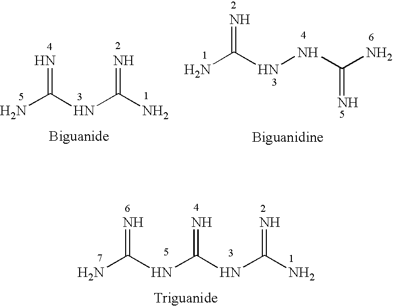

In one or more preferred embodiments, the guanide, biguanide, biguanidine or triguanide, provide bi-polar configurations of cationic and hydrophobic domains within a single molecule.

Examples of guanides, biguanides, biguanidines and triguanides that are currently been used as antibacterial agents include chlorhexidine and chlorohexidine salts, analogs and derivatives, such as chlorhexidine acetate, chlorhexidine gluconate and chlorhexidine hydrochloride, picloxydine, alexidine and polihexanide. Other examples of guanides, biguanides, biguanidines and triguanides that can conceivably be used according to the present invention are chlorproguanil hydrochloride, proguanil hydrochloride (currently used as antimalarial agents), metformin hydrochloride, phenformin and buformin hydrochloride (currently used as antidiabetic agents).

In one or more embodiments, the cationic antimicrobial agent is a polymer.

Cationic antimicrobial polymers include, for example, guanide polymers, biguanide polymers, or polymers having side chains containing biguanide moieties or other cationic functional groups, such as benzalkonium groups or quarternium groups (e.g., quaternary amine groups). It is understood that the term “polymer” as used herein includes any organic material comprising three or more repeating units, and includes oligomers, polymers, copolymers, block copolymers, terpolymers, etc. The polymer backbone may be, for example a polyethylene, ploypropylene or polysilane polymer.

In one or more embodiments, the cationic antimicrobial polymer is a polymeric biguanide compound. When applied to a substrate, such a polymer is known to form a barrier film that can engage and disrupt a microorganism. An exemplary polymeric biguanide compound is polyhexamethylene biguanide (PHMB) salts. Other exemplary biguanide polymers include, but are not limited to poly(hexamethylenebiguanide), poly(hexamethylenebiguanide) hydrochloride, poly(hexamethylenebiguanide) gluconate, poly(hexamethylenebiguanide) stearate, or a derivative thereof. In one or more embodiments, the antimicrobial material is substantially water-insoluble.

Yet, in one or more embodiment, the antibiotic is a non-classified antibiotic agent, including, without limitation, aabomycin, acetomycin, acetoxycycloheximide, acetylnanaomycin, an actinoplanes sp. Compound, actinopyrone, aflastatin, albacarcin, albacarcin, albofungin, albofungin, alisamycin, alpha-R,S-methoxycarbonylbenzylmonate, altromycin, amicetin, amycin, amycin demanoyl compound, amycine, amycomycin, anandimycin, anisomycin, anthramycin, anti-syphilis immune substance, anti-tuberculosis imune substance, antibiotic from Eschericia coli, antibiotics from Streptomyces refuineus, anticapsin, antimycin, aplasmomycin, aranorosin, aranorosinol, arugomycin, ascofuranone, ascomycin, ascosin, Aspergillus flavus antibiotic, asukamycin, aurantinin, an Aureolic acid antibiotic substance, aurodox, avilamycin, azidamfenicol, azidimycin, bacillaene, a Bacillus larvae antibiotic, bactobolin, benanomycin, benzanthrin, benzylmonate, bicozamycin, bravomicin, brodimoprim, butalactin, calcimycin, calvatic acid, candiplanecin, carumonam, carzinophilin, celesticetin, cepacin, cerulenin, cervinomycin, chartreusin, chloramphenicol, chloramphenicol palmitate, chloramphenicol succinate sodium, chlorflavonin, chlorobiocin, chlorocarcin, chromomycin, ciclopirox, ciclopirox olamine, citreamicin, cladosporin, clazamycin, clecarmycin, clindamycin, coliformin, collinomycin, copiamycin, corallopyronin, corynecandin, coumermycin, culpin, cuprimyxin, cyclamidomycin, cycloheximide, dactylomycin, danomycin, danubomycin, delaminomycin, demethoxyrapamycin, demethylscytophycin, dermadin, desdamethine, dexylosyl-benanomycin, pseudoaglycone, dihydromocimycin, dihydronancimycin, diumycin, dnacin, dorrigocin, dynemycin, dynemycin triacetate, ecteinascidin, efrotomycin, endomycin, ensanchomycin, equisetin, ericamycin, esperamicin, ethylmonate, everninomicin, feldamycin, flambamycin, flavensomycin, florfenicol, fluvomycin, fosfomycin, fosfonochlorin, fredericamycin, frenolicin, fumagillin, fumifungin, funginon, fusacandin, fusafungin, gelbecidine, glidobactin, grahamimycin, granaticin, griseofulvin, griseoviridin, grisonomycin, hayumicin, hayumicin, hazymicin, hedamycin, heneicomycin, heptelicid acid, holomycin, humidin, isohematinic acid, karnatakin, kazusamycin, kristenin, L-dihydrophenylalanine, a L-isoleucyl-L-2-amino-4-(4′-amino-2′, 5′-cyclohexadienyl) derivative, lanomycin, leinamycin, leptomycin, libanomycin, lincomycin, lomofungin, lysolipin, magnesidin, manumycin, melanomycin, methoxycarbonylmethylmonate, methoxycarbonylethylmonate, methoxycarbonylphenylmonate, methyl pseudomonate, methylmonate, microcin, mitomalcin, mocimycin, moenomycin, monoacetyl cladosporin, monomethyl cladosporin, mupirocin, mupirocin calcium, mycobacidin, myriocin, myxopyronin, pseudoaglycone, nanaomycin, nancimycin, nargenicin, neocarcinostatin, neoenactin, neothramycin, nifurtoinol, nocardicin, nogalamycin, novobiocin, octylmonate, olivomycin, orthosomycin, oudemansin, oxirapentyn, oxoglaucine methiodide, pactacin, pactamycin, papulacandin, paulomycin, phaeoramularia fungicide, phenelfamycin, phenyl, cerulenin, phenylmonate, pholipomycin, pirlimycin, pleuromutilin, a polylactone derivative, polynitroxin, polyoxin, porfiromycin, pradimicin, prenomycin, prop-2-enylmonate, protomycin, pseudomonas antibiotic, pseudomonic acid, purpuromycin, pyrinodemin, pyrrolnitrin, pyrrolomycin, amino, chloro pentenedioic acid, rapamycin, rebeccamycin, resistomycin, reuterin, reveromycin, rhizocticin, roridin, rubiflavin, naphthyridinomycin, saframycin, saphenamycin, sarkomycin, sarkomycin, sclopularin, selenomycin, siccanin, spartanamicin, spectinomycin, spongistatin, stravidin, streptolydigin, streptomyces arenae antibiotic complex, streptonigrin, streptothricins, streptovitacin, streptozotocine, a strobilurin derivative, stubomycin, sulfamethoxazol-trimethoprim, sakamycin, tejeramycin, terpentecin, tetrocarcin, thermorubin, thermozymocidin, thiamphenicol, thioaurin, thiolutin, thiomarinol, thiomarinol, tirandamycin, tolytoxin, trichodermin, trienomycin, trimethoprim, trioxacarcin, tyrissamycin, umbrinomycin, unphenelfamycin, urauchimycin, usnic acid, uredolysin, variotin, vermisporin, verrucarin and analogs, salts and derivatives thereof.

In one or more embodiments, the antibiotic agent is a naturally occurring antibiotic compound. As used herein, the term “naturally-occurring antibiotic agent” includes all antibiotic that are obtained, derived or extracted from plant or vertebrate sources. Non-limiting examples of families of naturally-occurring antibiotic agents include phenol, resorcinol, antibiotic aminoglycosides, anamycin, quinines, anthraquinones, antibiotic glycopeptides, azoles, macrolides, avilamycin, agropyrene, cnicin, aucubin antibioticsaponin fractions, berberine (isoquinoline alkaloid), arctiopicrin (sesquiterpene lactone), lupulone, humulone (bitter acids), allicin, hyperforin, echinacoside, coniosetin, tetramic acid, imanine and novoimanine.

Ciclopirox and ciclopiroxolamine possess fungicidal, fungistatic and sporicidal activity. They are active against a broad spectrum of dermatophytes, yeasts, moulds and other fungi, such as trichophyton species, microsporum species, epidermophyton species and yeasts (candida albicans, candida glabrata, other candida species and cryptococcus neoformans). Some aspergillus species are sensitive to ciclopirox as are some penicillium. Likewise, ciclopirox is effective against many gram-positive and gram-negative bacteria (e.g., escherichia coli, proteus mirabilis, pseudomonas aeruginosa, staphylococcus and streptococcus species), as well as mycoplasma species, trichomonas vaginalis and actinomyces.

Plant oils and extracts which contain antibiotic agents are also useful. Non limiting examples of plants that contain agents include thyme, perilla, lavender, tea tree, terfezia clayeryi, Micromonospora, putterlickia verrucosa, putterlickia pyracantha, putterlickia retrospinosa, Maytenus ilicifolia, maytenus evonymoides, maytenus aquifolia, faenia interjecta, cordyceps sinensis, couchgrass, holy thistle, plantain, burdock, hops, echinacea, buchu, chaparral, myrrh, red clover and yellow dock, garlic and St. John's wort.

Mixtures of these antibiotic agents may also be employed according to the present invention.

Antiviral

In an embodiment of the present invention, the therapeutic agent is an antiviral agent. Suitable antiviral agents include but are not limited to acyclovir, gancyclovir, ribavirin, amantadine, rimantadine nucleoside-analog reverse transcriptase inhibitors, such as zidovudine, didanosine, zalcitabine, tavudine, lamivudine and vidarabine, non-nucleoside reverse transcriptase inhibitors, such as nevirapine and delavirdine, protease inhibitors, such as saquinavir, ritonavir, indinavir and nelfinavir, and interferons and derivatives, esters, salts and mixtures thereof.

The Keratolytic Agent

The term “keratolytic agent” refers herein to a compound which loosens and removes the stratum corneum of the skin, or alters the structure of the keratin layers of skin.

In an embodiment of the present invention keratinolytic agents (e.g., topical salicylates) remove hyperkeratotic lesions that harbor pathogens, improving the exposure of the infected skin surface to other topical treatments including antifungal, antibiotic, and antiviral agents which may optionally be coupled with use of antiseptic agents, cleansing agents, drying agents, debriding agents and the like.

Suitable keratolytic agents include alpha-hydroxy acids. Alfa hydroxy acids are keratolytic, and they are also capable of trapping moisture in the skin and initiating the formation of collagen. Suitable hydroxy acids include but are not limited to agaricic acid, aleuritic acid, allaric acid, altraric acid, arabiraric acid, ascorbic acid, atrolactic acid, benzilic acid, citramalic acid, citric acid, dihydroxytartaric acid, erythraric acid, galactaric acid, galacturonic acid, glucaric acid, glucuronic acid, glyceric acid, glycolic acid, gularic acid, gulonic acid, hydroxypyruvic acid, idaric acid, isocitric acid, lactic acid, lyxaric acid, malic acid, mandelic acid, mannaric acid, methyllacetic acid, mucic acid, phenyllacetic acid, pyruvic acid, quinic acid, ribaric acid, ribonic acid, saccharic acid, talaric acid, tartaric acid, tartronic acid, threaric acid, tropic acid, uronic acids, xylaric acid and derivatives, esters, salts and mixtures thereof.

Yet, another preferred keratolytic agent is urea, as well as derivatives thereof. Urea possesses both keratolytic and skin-hydration properties which are beneficial to the damaged tissue of the skin.

Another preferred group of keratolytic agents, suitable for inclusion in the therapeutic composition according to the present invention is beta-hydroxy acids, such as salicylic acid (o-hydroxybenzoic acid). Beta hydroxyl acids are keratolytic, and they are also have anti-inflammatory and antibacterial properties.

Short chain carboxylic acids (carboxylic acids having up to 6 carbon atoms in their skeleton) are also suitable for inclusion in the therapeutic composition as keratolytic agents. Examples of short chain carboxylic acid include, but are not limited to formic acid, acetic acid, propionic acid, butyric acid (Butanoic acid), valeric acid (pentanoic acid) and caproic acid (hexanoic acid).

Dicarboxylic Acid and Esters Thereof

In the context of the present invention, a dicarboxylic acid is an organic material, having two carboxylic acid moieties on its carbon atom skeleton. They have the general molecular formula HOOC—(CH₂)ₙ—COOH. Di-carboxylic acids having up to 6 carbon atoms in their skeleton are also suitable under the definition of short chain carboxylic acids having up to 6 carbon atoms in their skeleton. Non-limiting examples of suitable dicarboxylic acids are oxalic acid, malonic acid (propanedioic acid), succinic acid (butanedioic acid), glutaric acid (Pentanedioic acid) and adipic acid (Hexanedioic acid). Also suitable under the definition of short chain carboxylic acid are unsaturated short chain carboxylic acids, i.e., short chain carboxylic acids, having one or more double bonds in their carbon skeleton such as in the case of maleic acid and fumaric acid; and halogenated short chain carboxylic acids, such as fluoroethanoic acid (CH₂FCO₂H), chloroethanoic acid (CH₂ClCO₂H) and dichloroethanoic acid (CHCl₂CO₂H). Dicarboxylic acids, having between about 6 and about 14 carbon atoms in their carbon atom skeleton such as brassylic acid (n=11), thapsic acid (n=14), also possess keratolytic properties. Suitable dicarboxylic acid moieties include, but are not limited to, adipic acid, pimelic acid, suberic acid, azelaic acid, sebacic acid, 1,11-undecanedioic acid, 1,12-dodecanedioic acid, 1,13-tridecanedioic acid and 1,14-tetradecanedioic acid. Derivatives such as esters (mono- or di-), amides and anhydrides can also be used under the scope of the present invention.

Another group of keratolytic agents include phenol and substituted phenolic compounds. Such compounds are known to dissolve and loosen the intracellular matrix of the hyperkeratinized tissue. Dihydroxy benzene and derivatives thereof have been recognized as potent keratolytic agents. Resorcinol (m-dihydroxybenzene) and derivatives thereof are used in anti-acne preparations. Hydroquinone (p-dihydroxybenzene), besides its anti-pigmentation properties, is also keratolytic.

Vitamin A and its derivatives, such as retinol, retinal, retinoic acid, retinyl acetate, retinyl palmitate, retinyl ascorbate, isotretinoin, tazarotene, adapalene, 13-cis-retinoic acid, acitretin all-trans beta carotene, alpha carotene, lycopene, 9-cis-beta-carotene, lutein and zeaxanthin are another class of keratolytic agents, which alter the structure of the skin and promote peeling.

In certain embodiments, the keratolytic agent includes at least two keratolytic agents. The at least two or more keratolytic agents in the therapeutic composition, a safe and effective peeling agent is attained, which breaks down the keratin layer of the skin, where the microorganisms reside. As a result of such breaking down of the keratin layer, the microorganisms cannot further survive in the infected area. The combination of at least two keratolytic agents enables a selective breaking down of keratin in infected skin areas, while non-infected skin areas are not affected. This phenomenon is explained by the fact that the keratin layer in infected skin areas is deformed and thus it is more vulnerable to keratolytic disintegration. Furthermore, combining at least two keratolytic agents facilitates use of each agent in a substantially minimally-irritating concentration, thus decreasing the overall irritation of the therapeutic composition.

In one or more embodiments, the keratolytic agent includes at least two keratolytic agents, from different families of chemicals. Thus, in preferred embodiments of the present invention, the keratolytic agent includes at east two agents, from different chemical families, selected from the group consisting of: (1) an alpha-hydroxy acid; (2) a beta-hydroxy acid; (3) a short-chain carboxylic acid; (4) a hydroxyl benzene; (5) a vitamin A derivative; and (6) urea. As detailed above, each of these keratolytic agent families possess, in addition to their keratolytic property, additional therapeutically-beneficial feature, such as anti-inflammatory, skin hydration and antibacterial properties for readily contributing to the overall therapeutic benefit of the therapeutic composition.

Skin Penetration Enhancer

A “skin penetration enhancer”, also termed herein “penetration enhancer,” is an organic solvent, typically soluble in both water and oil. Examples of penetration enhancer include polyols, such as glycerol (glycerin), propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, hexylene glycol, other glycols, sulfoxides, such as dimethylsulfoxide (DMSO), dimethylformanide, methyl dodecyl sulfoxide, dimethylacetamide, dimethylisosorbide, monooleate of ethoxylated glycerides (with 8 to 10 ethylene oxide units), azone (1-dodecylazacycloheptan-2-one), 2-(n-nonyl)-1,3-dioxolane, esters, such as isopropyl myristate/palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; amides, such as acetamide oleates such as triolein; various alkanoic acids such as caprylic acid; lactam compounds, such as azone; alkanols, such as dialkylamino acetates, and admixtures thereof.

According to one or more embodiments, the penetration enhancer is a polyethylene glycol (PEG) or PEG derivative that is liquid at ambient temperature.

Hydrophobic Solvent

In some embodiments, the augmented anti-infective foamable composition includes a hydrophobic solvent. A “hydrophobic solvent” as used herein refers to a material having solubility in distilled water at ambient temperature of less than about 1 gm per 100 mL, more preferable less than about 0.5 gm per 100 mL, and most preferably less than about 0.1 gm per 100 mL. The identification of a “hydrophobic solvent”, as used herein, is not intended to characterize the solubilization capabilities of the solvent for any specific active agent or any other component of the foamable composition. Rather, such information is provided to aid in the identification of materials suitable for use as a part in the foamable compositions described herein.

In one or more embodiments, the hydrophobic organic carrier is selected from the group consisting of mineral oil, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, unsaturated or polyunsaturated oils, such as olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils; essential oils; and silicone oils, such as dimethicone, cyclomethicone, polyalkyl siloxanes, polyaryl siloxanes, polyalkylaryl siloxanes and polyether siloxane copolymers, polydimethylsiloxanes (dimethicones) and poly(dimethylsiloxane)-(diphenyl-siloxane) copolymers. Yet, and other hydrophobic solvent, as defined here, which is known in the art of cosmetic and pharmaceutical formulation can be used. Combinations of two or more hydrophobic solvents are anticipated.

Surface-Active Agent

In some embodiments, the composition further contains an emulsifier a surface-active agent or surfactant and such terms can be used interchangeably. Surface-active agents (also termed “surfactants”) include any agent linking oil and water in the composition, in the form of emulsion. A surfactant's hydrophilic/lipophilic balance (HLB) describes the emulsifier's affinity toward water or oil. HLB is defined for non-ionic surfactants. The HLB scale ranges from 1 (totally lipophilic) to 20 (totally hydrophilic), with 10 representing an equal balance of both characteristics. Lipophilic emulsifiers form water-in-oil (w/o) emulsions; hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average). In many cases a single surfactant may suffice. In other cases a combination of two or more surfactants is desired.

The appropriate surfactant or surfactant system is related to the vehicle and intended purpose. In general terms a combination of surfactants is usually preferable when the vehicle is an emulsion. In a waterless or substantially waterless environment, it has been discovered that the presence of a surfactant or combination of surfactants produces breakable forms of good quality. In a waterless or substantially waterless silicone in polyol environment, it has been discovered that the presence of a surfactant or combination of surfactants produces breakable foams of good quality. This is particularly surprising since most silicons are used as defoaming agents.

The generally thought considerations for oil-in-water emulsions of using a surfactant or surfactant combination with preferably a HLB value or average in or towards the lipophilic side of the scale may not be applicable for waterless or substantially waterless systems as described herein. Moreover, the physical nature and characteristics of the surfactant, for example whether it is solid, semi solid, waxy or liquid, its amount and its chemical nature, for example, whether it is an ester or ether, may all have an impact on the selection of a surfactant for a waterless or substantially waterless composition. Similarly whether the composition forms liquid crystals may also have some significance. The presence of liquid crystals may improve foam quality. It is possible to utilize a wide range of HLB or weighted HLB values in the waterless compositions of the present invention.

Thus, according to one or more embodiments the composition contains a single surface active agent having an HLB value between about 2 and 9, or more than one surface active agent and the weighted average of their HLB values is between about 2 and about 9.

According to one or more other embodiments the composition contains a single surface active agent having an HLB value between about 7 and 14, or more than one surface active agent and the weighted average of their HLB values is between about 7 and about 14.

According to one or more further embodiments the composition contains a single surface active agent having an HLB value between about 9 and about 19, or more than one surface active agent and the weighted average of their HLB values is between about 9 and about 19.

In one or more embodiments the at least one surface active agent is solid, semi solid or waxy.

In one or more embodiments the carrier or composition is capable of forming or tends to form liquid crystals.

Preferably, the composition of the present invention contains a non-ionic surfactant. Non-limiting examples of possible non-ionic surfactants include a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59; a polyoxyethylene alkyl ether, polyoxyethylene cetyl ether, polyoxyethylene palmityl ether, polyethylene oxide hexadecyl ether, polyethylene glycol cetyl ether, brij 38, brij 52, brij 56 and brij W1, a sucrose ester, a partial ester of sorbitol and its anhydrides, sorbitan monolaurate, sorbitan monolaurate a monoglyceride, a diglyceride, isoceteth-20 and mono-, di- and tri-esters of sucrose with fatty acids.

Non-limiting examples of non-ionic surfactants that have HLB of about 7 to about 12 include steareth 2 (HLB˜4.9); glyceryl monostearate/PEG 100 stearate (Av HLB˜11.2); stearate Laureth 4 (HLB˜9.7) and cetomacrogol ether (e.g., polyethylene glycol 1000 monocetyl ether). Exemplary stabilizing surfactants which may be suitable for use in the present invention are found below.

PEG-Fatty Acid Monoester Surfactants

| Chemical Name | Product example name | HLB |
|---|---|---|
| PEG-30 stearate | Myrj 51 | >10 |
| PEG-40 laurate | Crodet L40 (Croda) | 17.9 |
| PEG-40 oleate | Crodet O40 (Croda) | 17.4 |
| PEG-45 stearate | Nikkol MYS-45 (Nikko) | 18 |
| PEG-50 stearate | Myrj 53 | >10 |
| PEG-100 stearate | Myrj 59, Arlacel 165 (ICI) | 19 |

PEG-Fatty Acid Diester Surfactants:

| Chemical Name | Product example name | HLB |
|---|---|---|
| PEG-4 dilaurate | Mapeg 200 DL (PPG), Kessco | 7 |
| | PEG 200 DL (Stepan), | |
| | LIPOPEG 2-DL (Lipo Chem.) | |
| PEG-4 | distearate Kessco 200 DS | 5 |
| | (Stepan) | |
| PEG-32 dioleate | Kessco PEG 1540 DO | 15 |
| | (Stepan) | |
| PEG-400 dioleate) | Cithrol 4DO series (Croda | >10 |
| PEG-400 disterate | Cithrol 4DS series (Croda) | >10 |
| PEG-20 glyceryl oleate | Tagat O (Goldschmidt) | >10 |

Transesterification Products of Oils and Alcohols

| Chemical Name | Product example name | HLB |
|---|---|---|
| PEG-30 castor oil | Emalex C-30 (Nihon Emulsion) | 11 |
| PEG-40 hydrogenated | Cremophor RH 40 (BASF), | 13 |
| castor oil) | Croduret (Croda), Emulgin | |
| | HRE 40 (Henkel) | |

Polyglycerized Fatty Acids, such as:

| Chemical Name | Product example name | LB |
|---|---|---|
| Polyglyceryl-6 dioleate | Caprol 6G20 (ABITEC); PGO- | 8.5 |
| | 62 (Calgene), PLUROL | |
| | OLEIQUE CC 497 | |
| | (Gattefosse)Hodag | |

PEG-Sorbitan Fatty Acid Esters

| Chemical Name | Product example name | HLB |
|---|---|---|
| PEG-20 sorbitan | Tween 40 (Atlas/ICI), Crillet 2 | 16 |
| monopalmitate | (Croda) | |
| PEG-20 sorbitan | Tween-60 (Atlas/ICI), Crillet 3 | 15 |
| monostearate | (Croda) | |
| PEG-20 sorbitan | Tween-80 (Atlas/ICI), Crillet 4 | 15 |
| | (Croda) | |
| PEG-20 sorbitan | Tween-80 (Atlas/ICI), Crillet 4 | 15 |
| | (Croda) | |

Polyethylene Glycol Alkyl Ethers

| Chemical Name | Product example name | HLB |
|---|---|---|
| PEG-2 oleyl ether | oleth-2 Brij 92/93 (Atlas/ICI) | 4.9 |
| PEG-3 oleyl ether | oleth-3 Volpo 3 (Croda) | <10 |
| PEG-5 oleyl ether | oleth-5 Volpo 5 (Croda) | <10 |
| PEG-10 oleyl ether | oleth-10 Volpo 10 (Croda), Brij | 12 |
| | 96/97 (Atlas/ICI) | |
| PEG-20 oleyl ether | oleth-20 Volpo 20 (Croda), Brij | 15 |
| | 98/99 (Atlas/ICI) | |
| PEG-4 lauryl ether | laureth-4Brij 30 (Atlas/ICI) | 9.7 |
| PEG-23 lauryl ether | laureth-23Brij 35 (Atlas/ICI) | 17 |
| PEG-10 stearyl ether | Brij 76 (ICI) | 12 |
| PEG-2 cetyl ether | Brij 52 (ICI) | 5.3 |

Sugar Ester Surfactants

| Chemical Name | Product example name | HLB |
|---|---|---|
| Sucrose distearate | Sisterna SP50, Surfope 1811 | 11 |

Sorbitan Fatty Acid Ester Surfactants

| Chemical Name | Product example name | HLB |
|---|---|---|
| Sorbitan monolaurate | Span-20 (Atlas/ICI), Crill 1 | 8.6 |
| | (Croda), Arlacel 20 (ICI) | |
| Sorbitan monopalmitate | Span-40 (Atlas/ICI), Crill 2 | 6.7 |
| | (Croda), Nikkol SP-10 (Nikko) | |
| Sorbitan monooleate | Span-80 (Atlas/ICI), Crill 4 | 4.3 |
| | (Croda), Crill 50 (Croda) | |
| Sorbitan monostearate | Span-60 (Atlas/ICI), Crill 3 | 4.7 |
| | (Croda), Nikkol SS-10 (Nikko) | |

In one or more embodiments the surface active agent is a complex emulgator in which the combination of two or more surface active agents can be more effective than a single surfactant and provides a more stable emulsion or improved foam quality than a single surfactant. For example and by way of non-limiting explanation it has been found that by choosing say two surfactants, one hydrophobic and the other hydrophilic the combination can produce a more stable emulsion than a single surfactant. Preferably, the complex emulgator comprises a combination of surfactants wherein there is a difference of about 4 or more units between the HLB values of the two surfactants or there is a significant difference in the chemical nature or structure of the two or more surfactants.

Specific non limiting examples of surfactant systems are, combinations of polyoxyethylene alkyl ethers, such as Brij 59/Brij10; Brij 52/Brij 10; Steareth 2/Steareth 20; Steareth 2/Steareth 21 (Brij 72/Brij 721); combinations of polyoxyethylene stearates such as Myrj 52/Myrj 59; combinations of sucrose esters, such as Surphope 1816/Surphope 1807; combinations of sorbitan esters, such as Span 20/Span 80; Span 20/Span 60; combinations of sucrose esters and sorbitan esters, such as Surphope 1811 and Span 60; combinations of liquid polysorbate detergents and PEG compounds, such as Tween 80/PEG-40 stearate; methyl glucose sesquistearate; polymeric emulsifiers, such as Permulen (TRI or TR2); liquid crystal systems, such as Arlatone (2121), Stepan (Mild RM1), Nikomulese (41) and Montanov (68) and the like.

In certain embodiments the surfactant is preferably a combination of steareth-2 and steareth-21; in certain other embodiments the surfactant is a combination of polysorbate 80 and PEG-40 stearate. In certain orther embodiments the surfactant is a combination of glyceryl monostearate/PEG 100 stearate. In certain other embodiments the surfactants is a combination of steareth 2 and methyl glucose sesquistearate. In certain other embodiments the surfactants is a combination of steareth 2 and cetearyl alcohol and cetearyl glucoside.

In certain cases, the surface active agent is selected from the group of cationic, zwitterionic, amphoteric and ampholytic surfactants, such as sodium methyl cocoyl taurate, sodium methyl oleoyl taurate, sodium lauryl sulfate, triethanolamine lauryl sulfate and betaines.

Many amphiphilic molecules can show lyotropic liquid-crystalline phase sequences depending on the volume balances between the hydrophilic part and hydrophobic part. These structures are formed through the micro-phase segregation of two incompatible components on a nanometer scale. Soap is an everyday example of a lyotropic liquid crystal. Certain types of surfactants tend to form lyotropic liquid crystals in emulsions interface (oil-in-water) and exert a stabilizing effect. Non limiting examples of surfactants with postulated tendency to form interfacial liquid crystals are: phospholipids, alkyl glucosides, sucrose esters, sorbitan esters. In certain embodiments of the present invention surfactants which tend to form liquid crystals may improve the quality of foams produced from compositions of the present invention.

In one or more embodiments of the present invention, the surface-active agent includes at least one non-ionic surfactant. Ionic surfactants are known to be irritants. Therefore, non-ionic surfactants are preferred in applications including sensitive tissue such as found in most mucosal tissues, especially when they are infected or inflamed. We have surprisingly found that non-ionic surfactants alone provide formulations and foams of good or excellent quality in the waterless and substantially waterless carriers and compositions of the present invention.

Thus, in a preferred embodiment, the surface active agent, the composition contains a non-ionic surfactant. In another preferred embodiment the composition includes a mixture of non-ionic surfactants as the sole surface active agent. Yet, in additional embodiments, the foamable composition includes a mixture of at least one non-ionic surfactant and at least one ionic surfactant in a ratio in the range of about 100:1 to 6:1. In further embodiments, surface active agent comprises a combination of a non-ionic surfactant and an ionic surfactant, at a ratio of between 1:1 and 20:1.

In selecting a suitable surfactant or combination thereof it should be borne in mind that the upper amount of surfactant that may be used may be limited by the shakability of the composition. In general terms, as the amount of non liquid surfactant is increased the shakability of the formulation reduces until a limitation point is reached where the formulation becomes non shakable. Thus in an embodiment of the present invention any effective amount of surfactant may be used provided the formulation remains shakable or at least flowable. In the present invention where it is desirable to use a high molecular weight solvent and more particularly significant amounts it may be helpful to include a liquid surfactant in addition to or in place of a more waxy surfactant and or to increase the level of the surfactant.

In certain embodiments of the present invention the amount of surfactant or combination of surfactants is between about 0.05% to about 20%; between about 0.05% to about 15%. or between about 0.05% to about 10%.

Polymeric Agent

The composition of the present invention contains a polymeric agent. It has been documented that the presence of a polymeric agent promotes a foam having fine bubble structure that does not readily collapse upon release from the pressurized aerosol can. The polymeric agent serves to stabilize the foam composition and to control drug residence in the target organ. Preferably, the polymeric agent is soluble or readily dispersible in the polar solvents, such as polyol or a mixture of a polyol and an additional polar solvent.

Non-limiting examples of polymeric agents that are soluble or readily dispersible in propylene glycol are Hydroxypropylcellulose and carbomer (homopolymer of acrylic acid is crosslinked with an allyl ether pentaerythritol, an allyl ether of sucrose, or an allyl ether of propylene, such as Carbopol® 934, Carbopol® 940, Carbopo® 941, Carbopol® 980 and Carbopol® 981.

Other polymeric agents are suitable for use according to the present invention provided that they are soluble or readily dispersible in the polyol; or in the mixture of a polyol and an additional polar solvent, on a case by case basis.

Exemplary polymeric agents include, in a non-limiting manner, naturally-occurring polymeric materials, such as locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, amine-bearing polymers such as chitosan; acidic polymers obtainable from natural sources, such as alginic acid and hyaluronic acid; chemically modified starches and the like, carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like.

Additional exemplary polymeric agents include semi-synthetic polymeric materials such as cellulose ethers, such as methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethylcellulose, carboxymethyl cellulose, carboxymethylcellulose carboxymethylhydroxyethylcellulose, and cationic celluloses. Polyethylene glycol, having molecular weight of 1000 or more (e.g., PEG 1,000, PEG 4,000, PEG 6,000 and PEG 10,000) also have gelling capacity and while they are considered as “additional polar solvents”, as detailed herein, they are also considered polymeric agents.

Mixtures of the above polymeric agents are contemplated.

The concentration of the polymeric agent should be selected so that the composition, after filling into aerosol canisters, is flowable, and can be shaken in the canister. In one or more embodiments, the concentration of the polymeric agent is selected such that the viscosity of the composition, prior to filling of the composition into aerosol canisters, is less than 12,000 CPs, and more preferably, less than 10,000 CPs.

Modulating Agent

The term modulating agent is used to describe an agent, which can improve the stability of or stabilize a foamable carrier or composition and or an active agent by modulating the effect of a substance or residue present in the carrier or composition.

In one or more embodiments the modulating agent is used in a water-in-oil or oil-in-water emulsion. In one or more other embodiments the modulating agent is used in a waterless emulsion.

In certain embodiments the substance or residue may for example be acidic or basic and alter in an aqueous or potentially alter pH in a waterless environment or it may be one or more metal ions which may act as a potential catalyst in an aqueous or non aqueous environment or it may also be a preservative or an antioxidant or an ionization agent.

In certain other embodiments the substance or residue may for example be acidic or basic and potentially alter an artificial pH in a waterless or substantially non aqueous environment or it may be one or more metal ions which may act as a potential catalyst in a waterless or substantially non aqueous environment.

In one or more embodiments the modulating agent is used to describe an agent which can affect pH in an aqueous solution. The agent can be any of the known buffering systems used in pharmaceutical or cosmetic formulations as would be appreciated by a man of the art. It can also be an organic acid, a carboxylic acid, a fatty acid an amino acid, an aromatic acid, an alpha or beta hydroxyl acid an organic base or a nitrogen containing compound.

In one or more further embodiments the modulating agent is used to describe an agent, which is a chelating or sequestering or complexing agent that is sufficiently soluble or functional in the solvent to enable it to “mop up” or “lock” metal ions.

In an embodiment modulating agent is used to describe an agent which can effect pH in an aqueous solution the term modulating agent more particularly means an acid or base or buffer system or combinations thereof, which is introduced into or is present in and acts to modulate the ionic or polar characteristics and any acidity or basesity balance of an emulsion carrier, composition, foamable carrier or foamable composition or resultant foam of the present invention.

In other embodiments modulating agent is used to describe an agent which can effect pH in an aqueous solution the term modulating agent more particularly means an acid or base or buffer system or combinations thereof, which is introduced into or is present in and acts to modulate the ionic or polar characteristics and any acidity or basesity balance of a waterless or substantially non aqueous carrier, composition, foamable carrier or foamable composition or resultant foam of the present invention.

The substance or residue can be introduced into the formulation from any one or more of the ingredients, some of which themselves may have acidic or basic properties. For example the polymer or solvent may contain basic residues in which case it may be desirable or beneficial to add an acid. Alternatively the surfactant may contain some acid residues in which case the addition of a base may be desirable and beneficial. In some cases more than one ingredient may contain residues which may ameliorate or compound their significance. For example if one ingredient provided weak acid residues and another stronger acid residues the pH in an emulsion environment should be lower. In contrast if one residue was acid and the other basic the net effect in the formulation maybe significantly reduced. In some circumstances the active ingredient may favor an acidic pH or more significantly may need to be maintained at a certain acidic pH otherwise it may readily isomerize, chemically react or breakdown, in which case introducing acidic components such as an acidic polymer might be of help. In an embodiment of the present invention sufficient modulating agent is added to achieve a pH in which the active agent is preferably stable. In another embodiment of the present invention sufficient modulating agent is added to achieve an artificial pH in which the active agent is preferably stable.

The terms pH, pKa, pKb, and buffers are used in classical measurements of an aqueous solution. Such measurements are artificial in a waterless environment. Waterless medium can be polar and protic, yet it does not conform to classical ionic behavior. Nevertheless, reference to and description below of such terms are made for convenience and clarity, since such terms are well defined and understood with reference to aqueous solutions and further due to the lack of an appropriate uniform way of describing and identifying the artificial or virtual pH, pK etc in a waterless environment in relation to the present invention. Although predictions of artificial pH can be made using dilution techniques of measurements of waterless formulations diluted in water they are formulation sensitive and specific and have to be carefully calibrated with complex formulas.

A buffer, as defined by Van Slyke [Van Slyke, J. Biol. Chem. 52, 525 (1922)], is “a substance which by its presence in solution increases the amount of acid or alkali that must be added to cause unit change in pH.” A buffer solution is a solution of a definite pH made up in such a way that this pH alters only gradually with the addition of alkali or acid. Such a solution typically consists of a solution of a salt of the weak acid in the presence of the acid itself, e.g., sodium citrate and citric acid. The pH of the solution is determined by the dissociation equilibrium of the free acid.

In an embodiment of the present invention, the moduating agent is a pH adjusting agent or a buffering agent. Suitable buffering agents include but are not limited to acetic acid, adipic acid, calcium hydroxide, citric acid, glycine, hydrochloric acid, lactic acid, magnesium aluminometasilicates, phosphoric acid, sodium carbonate, sodium citrate, sodium hydroxide, sorbic acid, succinic acid, tartaric acid, and derivatives, salts and mixtures thereof.

An acid can be a strong acid or a weak acid. A strong acid is an acid, which is a virtually 100% ionized in solution. In contrast, a weak acid is one which does not ionize fully when it is dissolved in water. The lower the value for pKa, the stronger is the acid and likewise, the higher the value for pKa, the weaker is the acid.

A base can be a strong base or a weak base. A strong base is something, which is fully ionic with 100% hydroxide ions. In contrast, a weak base is one which does not convert fully into hydroxide ions in solution. The lower the value for pKb, the stronger is the base and likewise, the higher the value for pKb the weaker is the base.

In one or more preferred embodiments, the modulating agent in a chelating angent and the chelating agent is selected from the group consisting of ethylenediaminetetraacetic acid (EDTA), diethylenetriaminepentaacetic acid (DTPA), hydroxyethylenediaminetriacetic acid (HEDTA), nitrilotriacetic acid (NTA), O,O′-bis(2-aminoethyl)ethyleneglycol-N,N,N′,N′-tetraacetic acid (EGTA), trans-1,2-diaminocyclohexane-N,N,N′,N′-tetraacetic acid (CyDTA) or a pharmaceutically acceptable salt thereof (normally as a sodium salt), more preferably EDTA, HEDTA and their salts; most preferably EDTA and its salts.

In one or more embodiments of the present invention a preferred non-limiting example of the chelating agent is EDTA. Typically, the chelating and sequestering agent is present in the composition at a level of up to about 5.0%, preferably 1.0 percent, by weight, of the composition.

In one or more embodiments of the present invention the modulating agent may also be a preservative or an antioxidant or an ionization agent. Any preservative, antioxidant or ionization agents suitable for pharmaceutical or cosmetic application may be used. Non limiting examples of antioxidants are tocopherol succinate, propyl galate, butylated hydroxy toluene and butyl hydroxy anisol. Ionization agents may be positive or may be negative depending on the environment and the active agent or composition that is to be protected. Ionization agents may for example act to protect or reduce sensitivity of active agents such that for example it is less likely to react or associate with something else. Non-limiting examples of positive ionization agents are benzyl conium chloride, and cetyl pyridium chloride. Non-limiting examples of negative ionization agents are sodium lauryl sulphate, sodium lauryl lactylate and phospholipids.

In another aspect, the active agents are ideally so far as is possible delivered onto the skin or within a body cavity at a pH of from about 3.5 to about 7.5, more preferably at a pH of about 4.5 to about 6.5. Thus in another aspect of the invention the modulating agents may help to create in a waterless or substantially waterless environment an artificial pH more suited to the skin or mucosal membrane requirements.

Propellants

Examples of suitable propellants include volatile hydrocarbons such as butane, propane, isobutane and fluorocarbon gases, or mixtures thereof.

In certain embodiments, fluorohydrocarbon propellants, other than chloro-fluoro carbons (CMCs) which are non-ozone-depleting propellants, are particularly useful in the production of a non-flammable foamable composition.

Such propellants include, but are not limited to hydrofluorocarbon (HFC) propellants, that contain no chlorine atoms, and as such, falls completely outside concerns about stratospheric ozone destruction by chlorofluorocarbons or other chlorinated hydrocarbons. Exemplary non-flammable propellants according to this aspect of the invention include propellants made by DuPont under the registered trademark Dymel, such as 1,1,1,2 tetrafluorethane (Dymel 134), and 1,1,1,2,3,3,3 heptafluoropropane (Dymel 227), 1,1, difluoro ethane (Dymel 152) and 1,1,1,3,3,3 hexafluoropropane. HFCs possess Ozone Depletion Potential of 0.00 and thus, they are allowed for use as propellant in aerosol products.

The propellant makes up about 5-25 wt % of the foamable composition. Aerosol propellants are used to generate and administer the foamable composition as a foam. The total composition including propellant, foamable compositions and optional ingredients is referred to as the foamable composition.

Foam Adjuvant

Optionally, a foam adjuvant is included in the foamable carriers of the present invention to increase the foaming capacity of surfactants and/or to stabilize the foam. In one or more embodiments of the present invention, the foam adjuvant agent includes fatty alcohols having 15 or more carbons in their carbon chain, such as cetyl alcohol and stearyl alcohol (or mixtures thereof). Other examples of fatty alcohols are arachidyl alcohol (C20), behenyl alcohol (C22), 1-triacontanol (C30), as well as alcohols with longer carbon chains (up to C50). Fatty alcohols, derived from beeswax and including a mixture of alcohols, a majority of which has at least 20 carbon atoms in their carbon chain, are especially well suited as foam adjuvant agents. The amount of the fatty alcohol required to support the foam system is inversely related to the length of its carbon chains. Foam adjuvants, as defined herein are also useful in facilitating improved spreadability and absorption of the composition.

In one or more embodiments of the present invention, the foam adjuvant agent includes fatty acids having 16 or more carbons in their carbon chain, such as hexadecanoic acid (C16) stearic acid (C18), arachidic acid (C20), behenic acid (C22), octacosanoic acid (C28), as well as fatty acids with longer carbon chains (up to C50), or mixtures thereof. As for fatty alcohols, the amount of fatty acids required to support the foam system is inversely related to the length of its carbon chain.

Optionally, the carbon atom chain of the fatty alcohol or the fatty acid may have at least one double bond. A further class of foam adjuvant agent includes a branched fatty alcohol or fatty acid. The carbon chain of the fatty acid or fatty alcohol also can be substituted with a hydroxyl group, such as 12-hydroxy stearic acid.

Additional Active Agent

Since fungal infection is often associated with additional conditions, such as inflammation and infection by other microorganisms (other than fungi or yeast), a combination of an antifungal agent, and an additional active agent, suitable for the treatment of the underlying disorder or another disorder which substantially concurrently occurs in the same patient is useful for simultaneous therapy of the patient's condition.

Suitable active agents include but are not limited to active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin A, vitamin A derivatives, vitamin B, vitamin B derivative, vitamin C, vitamin C derivatives, vitamin D, vitamin D derivatives, vitamin E, vitamin E derivatives, vitamin F, vitamin F derivatives, vitamin K, vitamin K derivatives, wound healing agents and wart removers.

Additional Component

In an embodiment of the present invention, a composition of the present invention includes one or more additional components. Such additional components include but are not limited to anti perspirants, anti-static agents, bulking agents, cleansers, colorants, conditioners, deodorants, diluents, dyes, emollients, fragrances, hair conditioners, humectants, occlusive agents, oils, pearlescent aids, perfuming agents, permeation enhancers, pH-adjusting agents, preservatives, protectants, skin penetration enhancers, softeners, solubilizers, sunscreens, sun blocking agents, sunless tanning agents, viscosity modifiers and vitamins. As is known to one skilled in the art, in some instances a specific additional component may have more than one activity, function or effect.

In an embodiment of the present invention, the additional component is an emollient. Suitable emollients include but are not limited to mineral oil, lanolin oil, coconut oil, cocoa butter, olive oil, aloe vera extract, jojoba oil, castor oil, fatty acids, fatty alcohols, diisopropyl adipate, hydroxybenzoate esters, benzoic acid esters of C9 to C15 alcohols, isononyl iso-nonanoate, silicone oils, polyethers, C12 to C15 alkyl benzoates, oleic acid, stearic fatty acid, cetyl alcohols, hexadecyl alcohol, dimethyl polysiloxane, polyoxypropylene cetyl ether, polyoxypropylene butyl ether, and derivatives, esters, salts and mixtures thereof.

In an embodiment of the present invention, the additional component is a humectant. Suitable humectants include but are not limited to guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, polyhydroxy alcohol, sorbitol, glycerol, hexanetriol, propylene glycol, butylene glycol, hexylene glycol, a hexylene glycol derivative, polyethylene glycol, a sugar, a starch, a sugar derivative, a starch derivative, alkoxylated glucose, hyaluronic acid, lactamide monoethanolamine, acetamide monoethanolamine and derivatives, esters, salts and mixtures thereof.

In an embodiment of the present invention, the additional component is a preservative. Suitable preservatives include but are not limited to C12 to C15 alkyl benzoates, alkyl p-hydroxybenzoates, aloe vera extract, ascorbic acid, benzalkonium chloride, benzoic acid, benzoic acid esters of C9 to C15 alcohols, butylated hydroxytoluene, castor oil, cetyl alcohols, chlorocresol, citric acid, cocoa butter, coconut oil, diazolidinyl urea, diisopropyl adipate, dimethyl polysiloxane, DMDM hydantoin, ethanol, fatty acids, fatty alcohols, hexadecyl alcohol, hydroxybenzoate esters, iodopropynyl butylcarbamate, isononyl iso-nonanoate, jojoba oil, lanolin oil, methylparaben, mineral oil, oleic acid, olive oil, polyethers, polyoxypropylene butyl ether, polyoxypropylene cetyl ether, potassium sorbate, silicone oils, sodium propionate, sodium benzoate, sodium bisulfite, sorbic acid, stearic fatty acid, vitamin E, vitamin E acetate and derivatives, esters, salts and mixtures thereof.

Composition and Foam Physical Characteristics and Advantages

A pharmaceutical or cosmetic composition manufactured using the foamable carrier of the present invention is very easy to use. When applied onto the afflicted body surface of mammals, i.e., humans or animals, it is in a foam state, allowing free application without spillage. Upon further application of a mechanical force, e.g., by rubbing the composition onto the body surface, it freely spreads on the surface and is rapidly absorbed.

The foamable composition of the present invention is stable, having an acceptable shelf-life of at least one year, or preferably, at least two years at ambient temperature, as revealed in accelerated stability tests. Organic carriers and propellants tend to impair the stability of emulsions and to interfere with the formation of a stable foam upon release from a pressurized container. It has been observed, however, that the foamable compositions according to the present invention are surprisingly stable. Following accelerated stability studies, they demonstrate desirable texture; they form fine bubble structures that do not break immediately upon contact with a surface, spread easily on the treated area and absorb quickly.

The composition should also be free flowing, to allow it to flow through the aperture of the container, e.g., and aerosol container, and create an acceptable foam.

Foam quality can be graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam, upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

Topically administrable foams are typically of quality grade E or G, when released from the aerosol container. Smaller bubbles are indicative of more stable foam, which does not collapse spontaneously immediately upon discharge from the container. The finer foam structure looks and feels smoother, thus increasing its usability and appeal.

As further aspect of the foam is breakability. The breakable foam is thermally stable, yet breaks under sheer force. Sheer-force breakability of the foam is clearly advantageous over thermally induced breakability. Thermally sensitive foams immediately collapse upon exposure to skin temperature and, therefore, cannot be applied on the hand and afterwards delivered to the afflicted area.

The foam of the present invention has several advantages:
(1) Breakability. The foam of the present invention is thermally stable. Unlike hydroalcoholic foam compositions of the prior art, the foam of the present invention is not “quick breaking”, i.e., it does not readily collapse upon exposure to body temperature environment. Sheer-force breakability of the foam is clearly advantageous over thermally induced breakability, since it allows comfortable application and well directed administration to the target area.
(2) Skin drying and skin barrier function. short chain alcohols are known to dry the skin and impair the integrity of the skin barrier. By contrast, including a film forming agent in the composition of the present invention foes not cause unwanted skin barrier damage.
(3) Irritability. Due to the lack of alcohol and improvement in skin barrier function, skin irritability is eliminated.

Another property of the foam is specific gravity, as measured upon release from the aerosol can. Typically, foams have specific gravity of less than 0.12 g/mL; or less than 0.10 g/mL; or less than 0.08 g/mL, depending on their composition and on the propellant concentration.

Fields of Pharmaceutical Applications

The foamable anti-infection (particularly antifungal) composition is suitable for treating any infected surface. In one or more embodiments, foamable carrier is suitable for administration to the skin, a body surface, a body cavity or mucosal surface, e.g., the mucosa of the nose, mouth, eye, ear, respiratory system, vagina or rectum (severally and interchangeably termed herein “target site”). Generally, the foamable antifungal composition of the present invention is suitable for treating a body surface, infected by a fungus and/or a yeast and or a bacteria and or a virus. In a preferred embodiment the main infection is an antifungal infection. In another preferred embodiment the foamable antifungal composition of the present invention is suitable for treating a body surface, where the infection site also has a secondary infection, which can be a different type of fungal infection, a bacterial infection or a viral infection or combinations thereof. Whilst usually less frequent the foamable antifungal/antiinfecive composition of the present invention is suitable for treating a body surface, where the prime infection at the infection site may be a bacterial or viral infection and the secondary infection can be a fungal infection.

In one or more embodiments of the present invention, the skin infection is a dermatophytosis (also termed tinea or ringworm). Dermatophytosis is caused by a closely related group of fungi known as dermatophytes which have the ability to utilise keratin as a nutrient source. The dermatophytosis can be found, among other areas, on the scalp, glabrous skin, and nails.

In one or more embodiments, the fungus is selected from the group consisting of epidermophyton floccosum, trichophyton rubrum, trichophyton interdigitale, trichophyton tonsurans, trichophyton violaceum, trichophyton concentricum, trichophyton schoenleinii, trichophyton soudanense, microsporum audouinii, microsporum ferrugineum, trichophyton mentagrophytes, trichophyton equinum, trichophyton erinacei, trichophyton verrucosum, microsporum canis, microsporum gypseum, microsporum nanum and microsporum cookie.

In an embodiment of the present invention, the foamable antifungal composition of the presenting invention is suitable for the treatment of a dermatophyte infection, selected from the group consisting of tinea corporis (ringworm of the body), tinea pedis (ringworm of the feet; athlete's foot), tinea unguium (ringworm of the nails), tinea capitis (ringworm of the scalp), tinea cruris (jock itch), tinea barbae (ringworm of the beard; barber's itch) and vesicular dermatitis.

In an embodiment of the present invention, the foamable antifungal composition is suitable for the treatment of a candida infection, also termed “candidiasis” and “moniliasis”. Typically, candidiasis occurs in moist, occluded, intertriginous areas of the skin, skin appendages, or mucous membranes.

Candida infections appear as well-demarcated, erythematous, sometimes itchy, exudative patches of varying size and shape. The lesions are usually rimmed with small red-based papules and pustules and occur in the axillae, inframammary areas, umbilicus, groin, and gluteal folds (eg, diaper rash [see Plate 113-3]); between the toes; and on the finger webs. Perianal candidiasis produces white macerated pruritus ani. Candidal paronychia begins around the nail as a painful red swelling that later develops pus. Subungual infections are characterized by distal separation of one or several fingernails (onycholysis) with white or yellow discoloration of the subungual area. Defects in cell-mediated immune responses (which, in children, are sometimes genetic) may lead to chronic mucocutaneous candidiasis (candidal granuloma), which is characterized by red, pustular, crusted, and thickened plaques resembling psoriasis, especially on the nose and forehead and invariably associated with chronic oral moniliasis. In immunodeficient patients, other more typical candidal lesions or systemic candidiasis may also occur. In an embodiment of the present invention, the foamable antifungal composition is suitable for the treatment of tinea versicolor, an infection characterized by multiple, usually asymptomatic, scaly patches varying from white to brown and caused by pityrosporum orbiculare.

In an embodiment of the present invention, the foamable antifungal composition is suitable for the treatment of genital candidiasis, which is symptomatic overgrowth of commensal yeasts on the mucosa of the vagina or penis.

In an embodiment of the present invention, the foamable antifungal composition is suitable for the treatment of pityriasis rosea.

In an embodiment of the present invention, the foamable antifungal composition is suitable for the treatment of dandruff.

In an embodiment of the present invention, the fungal infection concurrently involves hyperkeratosis (an excessive proliferation of the cells of the cornea), resulting in thickening of the horny layer of the skin. In further preferred embodiments the skin infection concurrently involves hyperkeratosis and an infection by a fungal microorganism.

In a preferred embodiment, the fungal infection is tinea pedis. Tinea pedis can involve any fungal strain, which infects the skin. Non-limiting examples of fungi are nondermatophtye, such as trichophyton, epidermophyton, microsporum. Other exemplary microorganisms that cause skin infection are malassezia furfur, corynebacterium minutissimum, and candida species.

In certain embodiments, the tinea pedis is located on the sole (vesicular type) or lateral aspects (moccasin type) of the foot and sometimes between the toes (interdigital type).

In one or more embodiments, the skin infection is diaper rash. In certain embodiments, the foamable antifungal composition further contains talcum powder or a substance with similar properties.

By combining an augmenting agent, an antifungal agent and an additional active agent in the foamable composition of the present invention, or by providing a kit comprising two or more aerosol canisters comprising an augmenting agent, an antifungal agent and an additional active agent respectively the foamable composition of the present invention is useful in treating an animal or a human patient having any one of a variety of dermatological disorders, including dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, ecthyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, grannuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo.

Likewise, the foamable composition of the present invention is suitable for treating a disorder of a body cavity or mucosal surface, e.g., the mucosa of the nose, mouth, eye, ear, respiratory system, vagina or rectum. Non limiting examples of such conditions include chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

Infections

Infections can for example be by invasion of skin structures by endogenous skin flora or by exogenous pathogenic organisms. Infections can be as a consequence of a parasitic attack. Common parasitic infections are creeping eruption, lice and scabies. Infections can be primary infections or secondary infections and both may be treated by the foam and foamable compositions of the present invention. More specifically, foam and foamable compositions of the present invention can be used in treating, ameliorating, retarding, reducing or preventing a dermatological, cosmetic or mucosal disorder or infection, wherein the disorder or infection is a primary or a secondary disorder or infection.

Non limiting examples of infections, are:
fungal, such as candidiasis (yeast infection), ringworm, tinea versicolor, dermatophytoses (ring worm, tinea), such as tinea barbae (beard), tinea capitis (scalp), tinea corporis (body), tinea cruris (groin), tinea manuum (hand), tinea pedis (athlete's foot), tinea unguium (nail); pityriasis versicolor; pityriasis versicolor (tinea versicolor), candidiasis, blastomycosis, chromoblastomycosis, mycetoma, mucormycosis, sporotrichosis, penicillium marneffei. fungal infections that may involve the skin by dissemination, such as aspergillosis, coccidioidomycosis, cryptococcosis, histoplasmosis, paracoccidioidomycosis. sometimes a fungal infection may affect mucous membranes, nails, or subcutaneous tissue, and there can be spread to deeper tissue and dissemination especially in immunosuppressed patients;
bacterial, such as cellulites, folliculitis, boils, and carbuncles, staphylococcal scalded skin syndrome, erysipelas, erythrasma, impetigo, paronychia, furunculosis, erysipelas, cellulitis, and ecthyma, erysipeloid, pitted keratolysis, trychomycosis secondary infections complicating pre-existing skin lesions, such as diabetic or other chronic superficial skin ulcers, burns, bites and stings, eczema, or as opportunistic infections after skin trauma say in immunocompromised patients; skin disorders of uncertain or mixed aetiology, such as acne and rosacea; systemic infections with cutaneous involvement such as anthrax, diphtheria, and mycetoma; and
viral, such as cold sores, herpes zoster (shingles), warts.
   Primary Infections

Primary skin infections have a characteristic clinical picture and disease course, are caused by a single pathogen, and usually affect normal skin. Primary infection can be topical, systemic or nerve. Non limiting examples of primary infections are brought below. Impetigo, folliculitis, and boils are common types. The most common primary skin pathogens are S aureus, b-hemolytic streptococci, and coryneform bacteria. These organisms usually enter through a break in the skin such as an insect bite. Many systemic infections involve skin symptoms caused either by the pathogen or by toxins; examples are measles, varicella, gonococcemia, and staphylococcal scalded skin syndrome. Dermatophytic fungi have a strong affinity for keratin and therefore invade keratinized tissue of the nails, hair, and skin.

Primary skin infections are mainly caused by staphylococci or streptococci. Staphylococci infections present as furuncles and carbuncles, superficial folliculitis, impetigo or rarely the Scalded Skin Syndrome. Streptococcal infections present as impetigo, ecthyma, erysipelas or cellulitis. Corynebacteria causes erythrasma, trichomycosis or pitted keratolysis. Gram-negative primary skin infections.

Secondary Infections

Secondary infections occur in skin that is already diseased. Due to the underlying disease, the clinical picture and course of these infections vary. Intertrigo and toe web infection are two of the various non limiting examples outlined below.

Intertrigo is most commonly seen in chubby infants or obese adults. In the skin fold, heat, moisture, and rubbing produce erythema, maceration, or even erosions. Overgrowth of resident or transient flora may produce this problem.

Acute infectious eczematoid dermatitis arises from a primary lesion such as a boil or a draining ear or nose, which are sources of infectious exudate. A hallmark of this disease is a streak of dermatitis along the path of flow of the discharge material. Coagulase-positive staphylococci are the organisms most frequently isolated.

Pseudofolliculitis of the beard, a common disorder, occurs most often in the beard area of black people who shave. The characteristic lesions are usually erythematous papules or, less commonly, pustules containing buried hairs. This occurs when a strongly curved hair emerging from curved hair follicles reenters the skin to produce an ingrown hair. Gram-positive microorganisms that belong to the resident flora are associated with this disordera clear illustration of the opportunism of nonpathogenic bacteria when the host defense is impaired.

More examples are cute infectious eczematoid dermatitis, infectious eczematoid dermatitis, pustular acne, pustular psoriasis, infected seborrheic dermatitis, infected contact dermatitis (including poison ivy), infected excoriations, and bacterial super-infections of fungal or viral infections.

The disease commonly referred to as athlete's foot has traditionally been regarded as strictly a fungal infection. This assumption has been revised, however, because fungi often cannot be recovered from the lesions throughout the disease course. Researchers now believe that the dermatophytes, the first invaders, cause skin damage that allows bacterial overgrowth, which promotes maceration and hyperkeratosis. The fungi, through the production of antibiotics, then create an environment that favors the growth of certain coryneform bacteria and Brevibacterium. Proteolytic enzymes, which are produced by some of these bacteria, may aggravate the condition. If the feet become superhydrated, resident Gram-negative rods become the predominant flora, and the toe webs incur further damage. The fungi are then eliminated either by the action of antifungal substances of bacterial origin or by their own inability to compete for nutrients with the vigorously growing bacteria.

Secondary skin infections, may also benefit from the inclusion in the formulations of the present invention of one or more of cleasing agents, antiseptic agents such as chlorhexidine and drying agents (eg aluminium chloride and other aluminium salts and complexes; hygroscopic agents or polymeric agents that mop up moisture), which help to inhibit overgrowth for example of opportunistic bacteria in foot, perineal and axillary areas. Like wise the inclusion of a debriding agent or its use in parallel may assist in removing a source of infection and minimizes its spread.

In an embodiment of the present invention, the foamable anti-infection and augmentation foams and foamable compositions may be applied to a target for the treatment of any one or more of the aforesaid infections as will be appreciated by a person skilled in the art.

Formulation of emulsion foam is a very delicate balance between the functional inactive ingredients, excipients, which contribute to droplet size, separating film, viscosity and stability. In order to assure accurate and continuous foam actuation, the Foam Formulation should be liquid and shakable in the canister, otherwise it will not flow easily and completely towards and through the valve. In the context of significant levels of silicone in foamable formulations it is possible as an exception for the composition to be marginally or apparently non shakable whilst the composition has a sufficient degree of flowability under pressure of the propellant that it is possible to obtain a good quality of foam.

In a preferred embodiment the emulsion composition should exhibit pseudoplastic rheological behavior.

By selective use of appropriate stabilizing surfactant, co-surfactants and optionally stabilizing polymers, in the silicone emulsion compositions and in the aqueous emulsion compositions of the present invention can be stabilized For example the foamable compositions, in the Examples presented below are able to produce good or excellent quality foams.

Methodology

A general procedure for preparing foamable compositions is set out in WO 2004/037225, which is incorporated herein by reference.

The emollient formulas of the present invention may be made in the following general methodology set out below with appropriate adjustments for each formulation as will be appreciated by someone skilled in the art.

Emulsion Foam

1. Mix oily phase ingredients and heat to 75 C to melt all ingredients and obtain homogeneous mixture.
2. Mix polymers in water with heating or cooling as appropriate for specific polymer.
3. Add all other water soluble ingredients to water-polymer solution and heat to 75 C.
4. Add slowly internal phase to external phase at 75 C under vigorous mixing and homogenize to obtain fine emulsion.
5. Cool to below 40 C and add sensitive ingredients with mild mixing.
6. Cool to room temperature.

The waterless formulas of the present invention may be made in the following general methodology set out below with appropriate adjustments for each formulation as will be appreciated by someone skilled in the art.

Waterless Foam

1. Dissolve the polymers in the main solvent with heating or cooling as appropriate for specific polymer. Add the all other ingredients and heat to 75° C. to melt and dissolve the various ingredients.
2. Cool to below 40° C. and add sensitive ingredients with mild mixing.
3. Cool to room temperature.
   Silicone in Glycol Emulsion
1. Mix main solvent emulsifiers and foam adjuvants and heat to 75° C. to melt and dissolve the various ingredients with vigorous mixing.
2. Homogenize the formulation with vigorous mixing.
3. Add the silicones at 60° C. with vigorous mixing.
4. Cool to below 40° C. and add sensitive ingredients with mild mixing.
   Formulations with HPMC

This methodology is suitable, for all formulations described comprising HPMC (Where the formulation is without polymer the production starts at section 2).
5. Dissolve the polymers in the main solvent with heating or cooling as appropriate for specific polymer and with vigorous mixing.
6. Add to main solvent emulsifiers and foam adjuvants and heat to 75° C. to melt and dissolve the various ingredients with vigorous mixing.
7. Homogenize the formulation with vigorous mixing.
8. Add the silicones if any at 60° C. with vigorous mixing.
9. Cool to below 40° C. and add sensitive ingredients with mild mixing.
10. Cool to room temperature.
   Formulations with ASOS

This methodology is suitable for all formulation comprising ASOS.

1. Add to main solvent emulsifiers and foam adjuvants and heat to 75° C. to melt and dissolve the various ingredients with vigorous mixing.

2. Homogenize the formulation with vigorous mixing.

3. Add the silicones if any at 60° C. with vigorous mixing.

4. Cool to below 40° C. and add sensitive ingredients with mild mixing.

5. Cool to room temperature.

Formulations with Carbopol

This methodology is suitable, for all formulation described comprising Carbopol.

1. Separate part from the solvent and add the carbopol.

2. Homogenize the carbopol at RT for few minutes until complete.

3. Add to the rest of main solvent emulsifiers and foam adjuvants and heat to 75° C. to melt and dissolve the various ingredients with vigorous mixing.

4. Homogenize the formulation with vigorous mixing.

5. Add the silicones if any at 60° C. with vigorous mixing.

6. Cool to below 40° C. and mix with carbopol mixture with vigorous mixing.

7. Cool to room temperature.

The canisters are then filled with the above waterless formula, sealed and crimped with a valve and pressurized with the propellant. For example,

1. Each aerosol canister 35×70 mm is filled with 30±5% g of the composition.

2. Each canister is closed with an aerosol valve, using a vacuum crimping machine.

3. Propellant (mix of propane, butane and isobutane) is added to each of the canisters.

Tests

By way of non limiting example the objectives of hardness, collapse time and FTC stability tests are briefly set out below as would be appreciated by a person of the art.

Hardness

LFRA100 instrument is used to characterize hardness. A probe is inserted into the test material. The resistance of the material to compression is measured by a calibrated load cell and reported in units of grams on the texture analyzer instrument display. Preferably at least three repeat tests are made. The textural characteristics of a dispensed foam can affect the degree of dermal penetration, efficacy, spreadability and acceptability to the user. The results can also be looked at as an indicator of softness. Note: the foam sample is dispensed into an aluminum sample holder and filled to the top of the holder.

Collapse Time

Collapse time (CT) is examined by dispensing a given quantity of foam and photographing sequentially its appearance with time during incubation at 36° C. It is useful for evaluating foam products, which maintain structural stability at skin temperature for at least 1 min.

Viscosity

Viscosity is measured with Brookfield LVDV-II+PRO with spindle SC4-25 at ambient temperature and 10, 5 and 1 RPM. Viscosity is usually measured at 10 RPM. However, at about the apparent upper limit for the spindle of ˜>50,000CP, the viscosity at 1 RPM may be measured, although the figures are of a higher magnitude.

Stock Compositions

Non-limiting examples of how stock solutions are made up with and without API. Other stock solutions may be made using the same methodology by simply varying adding or omitting ingredients as would be appreciated by one of the ordinary skills in the art.

### EXAMPLES

The invention is described with reference to the following examples. This invention is not limited to these examples and experiments. Many variations will suggest themselves and are within the full intended scope of the appended claims.

It is difficult to stabilize compositions with azelaic acid since azelaic acid is a solid and significant quantities of it are required. Nevertheless, azelaic acid particles are readily re-suspendable upon slight hand shaking. In a number of examples azelaic acid formulations produced foams of good quality with beneficial physical properties such as desired hardness and a reasonable collapse time during which the expanded foam remains substantially intact.

### Examples

### Emollient Emulsions

### Example 1

### Oil-in-Water Foamable Compositions Including an Antifungal Agent and Urea

| | U-T1 | U-T2 | U-M2 | U-C1 |
|---|---|---|---|---|
| | % w/w | % w/w | % w/w | % w/w |
| Urea (Keratolytic agent) | 10.00 | 10.00 | 10.00 | 10.00 |
| Terbinafine HCL | 1.00 | 2.00 | | |
| (Antifungal agent) | | | | |
| Miconazole (Antifungal agent) | | | 2.00 | |
| Ciclopirox-olamine | | | | 1.00 |
| (Antifungal agent) | | | | |
| Mineral oil (Hydrophobic solvent) | 5.60 | 5.60 | 5.60 | 5.60 |
| Isopropyl myristate | 5.60 | 5.60 | 5.60 | 5.60 |
| (Hydrophobic solvent) | | | | |
| Glyceryl monostearate | 0.45 | 0.45 | 0.45 | 0.45 |
| (Non-ionic surfactant) | | | | |
| Sorbitan monostearate | 0.60 | 0.60 | 0.60 | 0.60 |
| (Non-ionic surfactant) | | | | |
| Stearyl alcohol (Foam adjuvant) | 0.85 | 0.85 | 0.85 | 0.85 |
| Xanthan gum (Polymeric agent) | 0.26 | 0.26 | 0.26 | 0.26 |
| Hydroxypropyl methylcellulose | 0.26 | 0.26 | 0.26 | 0.26 |
| (Polymeric agent) | | | | |
| Polysorbate 60 (Non-ionic | 0.90 | 0.90 | 0.90 | 0.90 |
| surfactant) | | | | |
| PEG-40 stearate (Non-ionic | 2.60 | 2.60 | 2.60 | 2.60 |
| surfactant) | | | | |
| Cocamidopropyl betaine | 0.40 | 0.40 | | |
| (Ionic surfactant) | | | | |
| Preservative | 0.30 | 0.30 | 0.30 | 0.30 |
| Water pure | To 100 | To 100 | To 100 | To 100 |

Notes:
The liquefied or gas propellant can be added at a concentration of about 3% to about 25%.

The physical properties of formulation U-T2 are as follows:

| | |
|---|---|
| pH | 5.30 |
| Visual inspection of the pressurized | Homogeneous |
| composition (glass container) | |
| Centrifugation 10,000 RPM | Stable |
| Foam quality | Excellent |
| Density | 0.0429 |
| Viscosity, cP | 1465 |

Foamable composition U-T2 was found highly efficacious in treating dermatophite infections.

### Example 2

### Efficacy of a Foamable Composition Including Terbinafine and Urea

Two patients with moderate to severe tinea pedis, characterized by thickened keratinous layer, scaling and sogginess of the skin, at the spaces between the toes. The patients received treatment with foamable composition U-T2, once a day for 7 days. Surprisingly, in both patients there was a remarkable improvement, as observed visually after three days of treatment. After 3 days, full clearance of the infection was recorded in both patients. Visual observation also revealed clearance of the scales and thickened skin, which was interpreted as the effect of the keratolytic agent.

The patients remarked that the treatment was highly convenient and that the application of the foam onto the afflicted areas was easy. The foam reached the skin folds and spaces between the toes easily, and there was no need for extensive rubbing in order to attain absorption of the foam into the skin.

For comparison purposes, it is noted that typical treatment of tinea pedis with a terbinafine cream requires between 14 and 21 days of twice-daily administration.

### Example 3

### Oil-in-Water Foamable Compositions Including an Antifungal Agent and Either Azelaic Acid or Retinoic Acid

| | R-T1 | R-I1 | A-I1 | A-C1 |
|---|---|---|---|---|
| | % w/w | % w/w | % w/w | % w/w |
| Retinoic acid (Keratolytic agent) | 0.10 | 0.10 | | |
| Azelaic acid | | | 15.00 | 15.00 |
| Terbinafine HCL | 1.00 | | | |
| (Antifungal agent) | | | | |
| Itraconazole (Antifungal agent) | | 1.00 | 1.00 | |
| Ciclopirox-olamine | | | | 1.00 |
| (Antifungal agent) | | | | |
| Mineral oil (Hydrophobic solvent) | 5.60 | 5.60 | 5.60 | 5.60 |
| Isopropyl myristate | 5.60 | 5.60 | 5.60 | 5.60 |
| (Hydrophobic solvent) | | | | |
| Glyceryl monostearate | 0.45 | 0.45 | 0.45 | 0.45 |
| (Non-ionic surfactant) | | | | |
| Sorbitan monostearate | 0.60 | 0.60 | 0.60 | 0.60 |
| (Non-ionic surfactant) | | | | |
| Stearyl alcohol (Foam adjuvant) | 0.85 | 0.85 | 0.85 | 0.85 |
| Xanthan gum (Polymeric agent) | 0.26 | 0.26 | 0.26 | 0.26 |
| Hydroxypropyl methylcellulose | 0.26 | 0.26 | 0.26 | 0.26 |
| (Polymeric agent) | | | | |
| Polysorbate 60 | 0.90 | 0.90 | 0.90 | 0.90 |
| (Non-ionic surfactant) | | | | |
| PEG-40 stearate | 2.60 | 2.60 | 2.60 | 2.60 |
| (Non-ionic surfactant) | | | | |
| Cocamidopropyl betaine | 0.40 | 0.40 | | |
| (Ionic surfactant) | | | | |
| Preservative | 0.30 | 0.30 | 0.30 | 0.30 |
| Water pure | To 100 | To 100 | To 100 | To 100 |

Note: The liquefied or gas propellant can be added at a concentration of about 3% to about 25%.

### Example 4

### Oil-in-Water Foamable Compositions Comprising Octyl Dodecanol; Glyceryl Monostearate and an Antifungal Agent and a Keratolytic Agent

### Ex 4 Part A

| | KAF001-070311 | KAF002-070311 |
|---|---|---|
| Chemical Name | | |
| Glyceryl monostearate | 0.45 | 0.45 |
| Octyl dodecanol | 10.00 | 10.00 |
| Sorbitan stearate | 0.60 | 0.60 |
| Stearyl alcohol | 0.85 | 0.85 |
| Steareth-21 | 2.00 | 2.00 |
| PEG-40 stearate | 2.60 | 2.60 |
| Xanthane gum | 0.26 | 0.26 |
| Methylcellulose A4M | 0.30 | 0.30 |
| Polysorbate 80 | 0.90 | 0.90 |
| Purified water | 76.04 | 61.04 |
| Ciclopiroxolamine | 1.00 | 1.00 |
| Salicylic acid | 5.00 | |
| Urea | | 20.00 |
| | 100.00 | 100.00 |
| Propellant* | 8.00 | 8.00 |
| Results | | |
| APPEARANCE: Quality | Excellent | Excellent |
| Color | White | White |
| Odor | No Odor | No Odor |
| Shakability | Good | Good |
| Collapse time (sec.) | 280.00 | >300 |
| Hardness (g) | 13.12 | 12.36 |

*16% Propane, 81% Isobutane, 3% N-Butane

### Ex 4 Part B

| | KAF029-070321 | KAF030-070321 |
|---|---|---|
| Chemical Name | | |
| Glyceryl monostearate | 0.45 | 0.45 |
| Octyl dodecanol | 10.00 | 10.00 |
| Sorbitan stearate | 0.60 | 0.60 |
| Stearyl alcohol | 0.85 | 0.85 |
| Steareth-21 | 2.00 | 2.00 |
| PEG-40 stearate | 2.60 | 2.60 |
| Xanthane gum | 0.26 | 0.26 |
| Methylcellulose A4M | 0.30 | 0.30 |
| Polysorbate 80 | 0.90 | 0.90 |
| Purified water | 75.04 | 75.04 |
| Miconazole nitrate | 2.00 | |
| Terbinafine HCL | | 2.00 |
| Salicylic acid | 5.00 | 5.00 |
| | 100.00 | 100.00 |
| Propellant* | 8.00 | 8.00 |
| Results | | |
| APPEARANCE: Quality | FG | FG |
| Color | White | White |
| Odor | No Odor | No Odor |
| Shakability | Good | Good |

*16% Propane, 81% Isobutane, 3% N-Butane

Note:
The combinations of miconazole nitrate and salicylic acid or miconazole nitrate and terbinafine HCL in the formulations apparently reduced the quality of the resultant foam (See, Ex 4 Part B). This phenomenum was overcome by introducing these combinations into waterless formulations as can be seen below in the section on waterless examples. It was also overcome by introducing these combinations into oil-in-water foamable compositions comprising PPG stearyl ether and octyl dodecanol and buffered to pH 4.7. as can be seen in Example 8
The liquefied or gas propellant can be added at a concentration of about 3% to about 25%.

### Example 5

### Oil-in-Water Foamable Compositions Comprising Mineral Oil; Isopropyl Myrstate; Glyceryl Monostearate and an Antifungal Agent and a Keratolytic Agent

### Ex 5 Part A

| | KAF003-070311 | KAF004-070312 |
|---|---|---|
| Chemical Name | | |
| Glyceryl monostearate | 0.45 | 0.45 |
| Mineral oil | 5.60 | 5.60 |
| Isopropyl myristate | 5.60 | 5.60 |
| Stearic acid | 0.85 | 0.85 |
| PEG-40 stearate | 2.60 | 2.60 |
| microcrystalline cellulose and | 2.00 | 2.00 |
| sodium carboxymethyl cellulose | | |
| Polysorbate 80 | 0.90 | 0.90 |
| Purified water | 75.00 | 60.00 |
| Miconazole nitrate | 2.00 | 2.00 |
| Salicylic acid | 5.00 | |
| Urea | | 20.00 |
| | 100.00 | 100.00 |
| Propellant* | 8.00 | 8.00 |
| Results | | |
| APPEARANCE: Quality | FG | Excellent |
| Color | White | White |
| Odor | No Odor | No Odor |
| Shakability | Good | Good |
| Collapse time (sec.) | n/m** | >300 |
| Hardness (g) | n/m** | 13.43 |

*16% Propane, 81% Isobutane 3% N-Butane
**n/m—not measured

### Ex 5 Part B

| | KAF005-070312 |
|---|---|
| Chemical Name | |
| Glyceryl monostearate | 0.45 |
| Mineral oil | 5.60 |
| Isopropyl myristate | 5.60 |
| Stearic acid | 0.85 |
| PEG-40 stearate | 2.60 |
| microcrystalline cellulose and | 2.00 |
| sodium carboxymethyl cellulose | |
| Polysorbate 80 | 0.90 |
| Purified water | 68.00 |
| Miconazole nitrate | 2.00 |
| Lactic acid | 12.00 |
| | 100.00 |
| Propellant* | 8.00 |
| Results | |
| APPEARANCE: Quality | Excellent |
| Color | White |
| Odor | No Odor |
| Shakability | Good |
| Collapse time (sec.) | >300 |
| Hardness (g) | 11.44 |

*16% Propane, 81% Isobutane, 3% N-Butane

Note:
The combination of miconazole nitrate and salicylic acid in the formulation apparently reduced the quality of the resultant foam. This phenomenum was overcome by introducing this combination into waterless formulations as can be seen below in the section on waterless examples. It was also overcome by introducing the combination into oil-in-water foamable compositions comprising PPG stearyl ether and octyl dodecanol and buffered to pH 4.7. as can be seen in Example 8.
The liquefied or gas propellant can be added at a concentration of about 3% to about 25%.

### Example 6

### Oil-in-Water Foamable Compositions Comprising Mineral Oil; Isopropyl Myrstate; Glyceryl Monostearate with Cocoamide DEA and an Antifungal Agent and a Keratolytic Agent

| | KAF006-070313 | KAF007-070318 |
|---|---|---|
| Chemical Name | | |
| Glyceryl monostearate | 0.45 | 0.45 |
| Mineral oil | 5.60 | 5.60 |
| Isopropyl myristate | 5.60 | 5.60 |
| Sorbitan stearate | 0.60 | 0.60 |
| Stearyl alcohol | 0.85 | 0.85 |
| PEG-40 stearate | 2.60 | 2.60 |
| Xanthane gum | 0.26 | 0.26 |
| Methyl | 0.26 | 0.26 |
| cellulose A4M | | |
| Polysorbate 60 | 0.90 | 0.90 |
| Cocoamide DEA | 0.40 | 0.40 |
| Purified water | 75.48 | 68.48 |
| Terbinafine HCL | 2.00 | 2.00 |
| Salicylic acid | 5.00 | |
| Lactic acid | | 12.00 |
| | 100.00 | 100.00 |
| Propellant* | 8.00 | 8.00 |
| Results | | |
| APPEARANCE: | FG | Excellent |
| Quality | | |
| Color | White | White |
| Odor | No Odor | No Odor |
| Shakability | Good | Good |
| Collapse time (sec.) | n/m** | >300 |
| Hardness (g) | n/m** | 17.53 |

*16% Propane, 81% Isobutane, 3% N-Butane
**n/m—not measured

Note:
The combination of terbinafine HCL and salicylic acid in the formulation apparently reduced the quality of the resultant foam. This phenomenum was overcome by introducing this combination into waterless formulations as can be seen below in the section on waterless examples. It was also overcome by introducing the combination into oil-in-water foamable compositions comprising PPG stearyl ether and octyl dodecanol and buffered to pH 4.7. as can be seen in Example 8.
The liquefied or gas propellant can be added at a concentration of about 3% to about 25%.

### Example 7

### Oil-in-Water Foamable Compositions Comprising Caprylic/Capric Triglycerides; DMI as a Penetration Enhancer and an Antifungal Agent and Azelaic Acid

| | | | KAF010- |
|---|---|---|---|
| | KAF008-070319 | KAF009-070319 | 070319 |
| Chemical Name | | | |
| Caprylic/Capric | 10.76 | 10.65 | 10.65 |
| Triglyceride | | | |
| Cetostearyl alcohol | 1.08 | 1.07 | 1.07 |
| Glyceryl stearate | 0.53 | 0.52 | 0.52 |
| Benzoic acid | 0.10 | 0.10 | 0.10 |
| PEG-40 Stearate | 2.80 | 2.77 | 2.77 |
| Methylcellulose A4M | 0.11 | 0.11 | 0.11 |
| Xanthan gum | 0.27 | 0.27 | 0.27 |
| Polysorbate 80 | 0.97 | 0.96 | 0.96 |
| Water, purified | 51.38 | 50.86 | 50.86 |
| Azelaic Acid | 14.85 | 14.70 | 14.70 |
| Dimethyl isosorbide | 5.39 | 5.34 | 5.34 |
| Propylene Glycol | 10.76 | 10.65 | 10.65 |
| Miconazole nitrate | | 2.00 | |
| Ciclopiroxolamine | 1.00 | | |
| Terbinafine HCL | | | 2.00 |
| | 100.00 | 100.00 | 100.00 |
| Propellant* | 8.00 | 8.00 | 8.00 |
| Results | | | |
| APPEARANCE: | Excellent | Excellent | Excellent |
| Quality | | | |
| Color | White | White | White |
| Odor | No Odor | No Odor | No Odor |
| Shakability | Good | Good | Good |
| Collapse time (sec.) | >300 | >300 | >300 |
| Hardness (g) | 26.88 | 29.55 | 34.17 |

*16% Propane, 81% Isobutane, 3% N-Butane

Note: The liquefied or gas propellant can be added at a concentration of about 3% to about 25%.

### Example 8

### Oil-in-Water Foamable Compositions Comprising PPG Stearyl Ether; Octyl Dodecanol; Polymeric Agents and an Antifungal Agent and a Keratolytic Agent with and Without Vitamin E

### Ex8 Part A

| | KAF042-070322 | KAF043-070322 |
|---|---|---|
| Chemical Name | | |
| PPG-15 stearyl ether | 5.69 | 5.28 |
| Octyldodecanol | 5.69 | 5.28 |
| steareth 21 | 1.52 | 1.40 |
| Laureth-4 | 2.09 | 1.94 |
| Ceteth-20 | 1.43 | 1.32 |
| Methylcellulose K100M | 0.24 | 0.22 |
| Xanthan gum | 0.24 | 0.22 |
| Water, purified | 73.06 | 67.58 |
| Benzyl alcohol | 0.95 | 0.88 |
| Propylene Glycol | 2.85 | 2.64 |
| Alpha Tocopherol | 0.05 | 0.04 |
| Clindamycin Phosphate | 1.20 | 1.20 |
| Salicylic acid | 5.00 | |
| Lactic acid | | 12.00 |
| | 100.00 | 100.00 |
| Sodium Hydroxide Solution 18% | To pH 4.7 | To pH 4.7 |
| Propellant* | 8.00 | 8.00 |
| Results | | |
| APPEARANCE: Quality | Excellent | Excellent |
| Color | White | White |
| Odor | No Odor | No Odor |
| Shakability | Good | Good |

*16% Propane, 81% Isobutane, 3% N-Butane

### Ex 8 Part B

| | KAF048-070325 | KAF049-070325 |
|---|---|---|
| Chemical Name | | |
| PPG-15 stearyl ether | 5.64 | 5.64 |
| Octyldodecanol | 5.64 | 5.64 |
| steareth 21 | 1.51 | 1.51 |
| Laureth-4 | 2.07 | 2.07 |
| Ceteth-20 | 1.42 | 1.42 |
| Methylcellulose K100M | 0.24 | 0.24 |
| Xanthan gum | 0.24 | 0.24 |
| Water, purified | 72.47 | 72.47 |
| Benzyl alcohol | 0.94 | 0.94 |
| Propylene Glycol | 2.83 | 2.83 |
| Miconazole nitrate | 2.00 | |
| Terbinafine HCL | | 2.00 |
| Salicylic acid | 5.00 | 5.00 |
| | 100.00 | 100.00 |
| Sodium Hydroxide Solution 18% | To pH 4.7 | To pH 4.7 |
| Propellant* | 8.00 | 8.00 |
| Results | | |
| APPEARANCE: Quality | Excellent | Excellent |
| Color | White | White |
| Odor | No Odor | No Odor |
| Shakability | Good | Good |

*16% Propane, 81% Isobutane, 3% N-Butane

Note: The liquefied or gas propellant can be added at a concentration of about 3% to about 25%.

Waterless Formulations

Note: it is predicted that the addition of a modulating agent with the active agents should have minimal effect on the physical characteristics of resultant foam.

Propylene Glycol (PG) Based Formulations

### Example 9

### Ex 9A) Propylene Glycol Foamable Compositions Comprising PG; a Polymeric Agent and An Antifungal Agent and a Keratolytic Agent

### Ex 9a) Part A

| | KAF011-070319 | KAF012-070319 |
|---|---|---|
| Chemical Name | | |
| Propylene glycol | 89.18 | 90.09 |
| Stearyl alcohol | 1.96 | 1.98 |
| Klucel EF | 1.96 | 1.98 |
| Laureth-4 | 1.96 | 1.98 |
| Glyceryl Monostearate/ | 2.94 | 2.97 |
| PEG 100 Stearate | | |
| Ciclopiroxolamine | | 1.00 |
| Terbinafine HCL | 2.00 | |
| | 100.00 | 100.00 |
| Propellant* | 8.00 | 8.00 |
| Results | | |
| APPEARANCE: Quality | Good | Good |
| Color | White | White |
| Odor | No Odor | No Odor |
| Shakability | Good | Good |
| Collapse time (sec.) | >300 | >300 |

*16% Propane, 81% Isobutane, 3% N-Butane

### Ex 9a) Part B

| | KAF013-070319 | KAF032-070322 |
|---|---|---|
| Chemical Name | | |
| Propylene glycol | 89.18 | 84.63 |
| Stearyl alcohol | 1.96 | 1.86 |
| Klucel EF | 1.96 | 1.86 |
| Laureth-4 | 1.96 | 1.86 |
| Glyceryl Monostearate/ | 2.94 | 2.79 |
| PEG 100 Stearate | | |
| Salicylic acid | | 5.00 |
| Miconazole nitrate | 2.00 | 2.00 |
| | 100.00 | 100.00 |
| Propellant* | 8.00 | 8.00 |
| Results | | |
| APPEARANCE: Quality | Good | Good |
| Color | White | White |
| Odor | No Odor | No Odor |
| Shakability | Good | Good |
| Collapse time (sec.) | >300 | n/m** |

*16% Propane, 81% Isobutane, 3% N-Butane
**n/m—not measured

### Ex 9b) Propylene Glycol Theoretical Foamable Compositions Comprising PG; a Polymeric Agent and an Antifungal Agent and a Keratolytic Agent

| | Theoretical | |
|---|---|---|
| Chemical Name | KAF033-070323 | KAF034-070323 |
| Propylene glycol | 85.54 | 84.63 |
| Stearyl alcohol | 1.88 | 1.86 |
| Klucel EF | 1.88 | 1.86 |
| Laureth-4 | 1.88 | 1.86 |
| Glyceryl Monostearate/PEG 100 | 2.82 | 2.79 |
| Stearate | | |
| Salicylic acid | 5.00 | 5.00 |
| Ciclopiroxolamine | 1.00 | |
| Terbinafine HCL | | 2.00 |
| | 100.00 | 100.00 |
| Propellant* | 8.00 | 8.00 |

*16% Propane, 81% Isobutane, 3% N-Butane

Note:
The liquefied or gas propellant can be added at a concentration of about 3% to about 25%.

### Example 10

### Propylene Glycol Foamable Compositions Comprising PG; a Polymeric Agent and an Antifungal Agent and Azelaic Acid

### Ex 10 Part A

| | KAF026-070321 | KAF027-070321 |
|---|---|---|
| Chemical Name | | |
| Propylene glycol | 80.08 | 80.99 |
| Stearyl alcohol | 1.76 | 1.78 |
| Klucel EF | 1.76 | 1.78 |
| Laureth-4 | 1.76 | 1.78 |
| Glyceryl Monostearate/ | 2.64 | 2.67 |
| PEG 100 Stearate | | |
| Azelaic acid | 10.00 | 10.00 |
| Ciclopiroxolamine | | 1.00 |
| Terbinafine HCL | 2.00 | |
| | 100.00 | 100.00 |
| Propellant* | 8.00 | 8.00 |
| Results | | |
| APPEARANCE: Quality | Good | Good |
| Color | White | White |
| Odor | No Odor | No Odor |
| Shakability | Good | Good |
| Collapse time (sec.) | 270.00 | 130.00 |
| Hardness (g) | | 61.35 |

*16% Propane, 81% Isobutane, 3% N-Butane

### Ex 10 Part B

| | KAF028-070321 |
|---|---|
| Chemical Name | |
| Propylene glycol | 80.08 |
| Stearyl alcohol | 1.76 |
| Klucel EF | 1.76 |
| Laureth-4 | 1.76 |
| Glyceryl Monostearate/PEG 100 Stearate | 2.64 |
| Azelaic acid | 10.00 |
| Miconazole nitrate | 2.00 |
| | 100.00 |
| Propellant* | 8.00 |
| Results | |
| APPEARANCE: Quality | Good |
| Color | White |
| Odor | No Odor |
| Shakability | Good |
| Collapse time (sec.) | 230.00 |

*16% Propane, 81% Isobutane, 3% N-Butane

Note: The liquefied or gas propellant can be added at a concentration of about 3% to about 25%.

### Example 11

### Propylene Glycol Foamable Compositions Comprising PG; a Polymeric Agent and an Antiviral Agent and a Keratolytic Agent

| | KAF041-070322 |
|---|---|
| Chemical Name | |
| Propylene glycol | 81.90 |
| Stearyl alcohol | 1.80 |
| Klucel EF | 1.80 |
| Laureth-4 | 1.80 |
| Glyceryl Monostearate/PEG 100 Stearate | 2.70 |
| Salicylic acid | 5.00 |
| Acyclovir | 5.00 |
| | 100.00 |
| Propellant* | 8.00 |
| Results | |
| APPEARANCE: Quality | Good |
| Color | White |
| Odor | No Odor |
| Shakability | Good |

*16% Propane, 81% Isobutane, 3% N-Butane

Note: The liquefied or gas propellant can be added at a concentration of about 3% to about 25%.

### Example 12

### Propylene Glycol Foamable Compositions Comprising PG; a Polymeric Agent and an Antibiotic Agent and a Keratolytic Agent

| | KAF044-070322 | KAF046-070325 |
|---|---|---|
| Chemical Name | | |
| Propylene glycol | 84.63 | 78.26 |
| Stearyl alcohol | 1.86 | 1.72 |
| Klucel EF | 1.86 | 1.72 |
| Laureth-4 | 1.86 | 1.72 |
| Glyceryl Monostearate/ | 2.79 | 2.58 |
| PEG 100 Stearate | | |
| Lactic acid | | 12.00 |
| Salicylic acid | 5.00 | |
| Mupirocine | 2.00 | 2.00 |
| | 100.00 | 100.00 |
| Propellant* | 8.00 | 8.00 |
| Results | | |
| APPEARANCE: Quality | Good | Good |
| Color | White | White |
| Odor | No Odor | No Odor |
| Shakability | Good | Good |

*16% Propane, 81% Isobutane, 3% N-Butane

Note: The liquefied or gas propellant can be added at a concentration of about 3% to about 25%.

### Example 13

### Ex 13a

### Propylene Glycol Foamable Compositions Comprising PG; Glycerin; DMI as a Penetration Enhancer and an Antifungal Agent and a Keratolytic Agent

### Ex 13a) Part A

| | KAF017-070319 | KAF018-070319 |
|---|---|---|
| Chemical Name | | |
| Propylene glycol | 45.08 | 45.54 |
| Glycerin anhydrous | 32.34 | 32.67 |
| Stearyl alcohol | 0.98 | 0.99 |
| Hydroxypropyl cellulose | 1.47 | 1.49 |
| Laureth-4 | 1.96 | 1.98 |
| Glyceryl Monostearate/ | 1.47 | 1.49 |
| PEG 100 Stearate | | |
| Dimethyl isosorbide | 14.70 | 14.85 |
| Ciclopiroxolamine | | 1.00 |
| Terbinafine HCL | 2.00 | |
| | 100.00 | 100.00 |
| Propellant* | 8.00 | 8.00 |
| Results | | |
| APPEARANCE: Quality | Good | Good |
| Color | White | White |
| Odor | No Odor | No Odor |
| Shakability | Good | Good |
| Collapse time (sec.) | 280.00 | 290.00 |
| Hardness (g) | 74.86 | 54.36 |

*16% Propane, 81% Isobutane, 3% N-Butane

### Ex 13a) Part B

| | KAF019-070319 | KAF038-070322 |
|---|---|---|
| Chemical Name | | |
| Propylene glycol | 45.08 | 42.78 |
| Glycerin anhydrous | 32.34 | 30.69 |
| Stearyl alcohol | 0.98 | 0.93 |
| Hydroxypropyl cellulose | 1.47 | 1.40 |
| Laureth-4 | 1.96 | 1.86 |
| Glyceryl Monostearate/ | 1.47 | 1.40 |
| PEG 100 Stearate | | |
| Dimethyl isosorbide | 14.70 | 13.95 |
| Salicylic acid | | 5.00 |
| Miconazole nitrate | 2.00 | 2.00 |
| | 100.00 | 100.00 |
| Propellant* | 8.00 | 8.00 |
| Results | | |
| APPEARANCE: Quality | Good | Good |
| Color | White | White |
| Odor | No Odor | No Odor |
| Shakability | Good | Good |
| Collapse time (sec.) | >300 | n/m** |

*16% Propane, 81% Isobutane, 3% N-Butane
**n/m—not measured

### Ex 13b

### Propylene Glycol Theoretical Foamable Compositions Comprising PG; Glycerin; DMI as a Penetration Enhancer and an Antifungal Agent and a Keratolytic Agent

| | Theoretical | |
|---|---|---|
| Chemical Name | KAF039-070322 | KAF040-070322 |
| Propylene glycol | 43.24 | 42.77 |
| Glycerin anhydrous | 31.02 | 30.69 |
| Stearyl alcohol | 0.94 | 0.93 |
| Hydroxypropyl cellulose | 1.41 | 1.40 |
| Laureth-4 | 1.88 | 1.86 |
| Glyceryl Monostearate/PEG 100 | 1.41 | 1.40 |
| Stearate | | |
| Dimethyl isosorbide | 14.10 | 13.95 |
| Salicylic acid | 5.00 | 5.00 |
| Ciclopiroxolamine | 1.00 | |
| Terbinafine HCL | | 2.00 |
| | 100.00 | 100.00 |
| Propellant* | 8.00 | 8.00 |

*16% Propane, 81% Isobutane, 3% N-Butane

Note: The liquefied or gas propellant can be added at a concentration of about 3% to about 25%.

### Example 14

### Propylene Glycol Foamable Compositions Comprising PG; Glycerin; DMI as A Penetration Enhancer and an Antifungal Agent and Azelaic Acid

| | KAF023- | KAF024- | KAF025- |
|---|---|---|---|
| | 070320 | 070320 | 070320 |
| Chemical Name | | | |
| Propylene glycol | 40.48 | 40.94 | 40.48 |
| Glycerin anhydrous | 29.04 | 29.37 | 29.04 |
| Stearyl alcohol | 0.88 | 0.89 | 0.88 |
| Hydroxypropyl cellulose | 1.32 | 1.34 | 1.32 |
| Laureth-4 | 1.76 | 1.78 | 1.76 |
| Glyceryl Monostearate/ | 1.32 | 1.34 | 1.32 |
| PEG 100 Stearate | | | |
| Dimethyl isosorbide | 13.20 | 13.35 | 13.20 |
| Azelaic acid | 10.00 | 10.00 | 10.00 |
| Miconazole nitrate | | | 2.00 |
| Ciclopiroxolamine | | 1.00 | |
| Terbinafine HCL | 2.00 | | |
| | 100.00 | 100.00 | 100.00 |
| Propellant* | 8.00 | 8.00 | 8.00 |
| Results | | | |
| APPEARANCE: Quality | Good | Good | Good |
| Color | White | White | White |
| Odor | No Odor | No Odor | No Odor |
| Shakability | Good | Good | Good |
| Collapse time (sec.) | >300 | 240.00 | 280.00 |

*16% Propane, 81% Isobutane, 3% N-Butane

Note: The liquefied or gas propellant can be added at a concentration of about 3% to about 25%.

Polyethylene Glycol (PEG) Based Formulations

PEG 400 can be substituted by other PEG,s like PEG 200 or PEG 600 or mixtures thereof and small amounts of higher molecular weight PEG be added if is appropriate to make the composition more viscous. Propylene glycol can be used instead of PEG. Mixtures of PEG(s) and PG can also be used.

### Example 15

### Ex 15a

### Polyethylene Glycol Foamable Compositions Comprising PEG 400; Polymer; and an Antifungal Agent and a Keratolytic Agent

### Ex 15a) Part A

| Chemical Name | | KAF014-070319 | KAF015-070319 |
|---|---|---|---|
| | Chemical Name | | |
| | Polyethylene glycol | 95.55 | 96.53 |
| | 400 | | |
| | Hydroxypropyl | 0.49 | 0.50 |
| | cellulose | | |
| | Steareth 2 | 1.96 | 1.98 |
| | Ciclopiroxolamine | | 1.00 |
| | Terbinafine HCL | 2.00 | |
| | | 100.00 | 100.00 |
| | Propellant* | 8.00 | 8.00 |
| | Results | | |
| | APPEARANCE: | Good | Good |
| | Quality | | |
| | Color | White | White |
| | Odor | No Odor | No Odor |
| | Shakability | Good | Good |
| | Collapse time (sec.) | >300 | >300 |

*16% Propane, 81% Isobutane, 3% N-Butane

### Ex 15a) Part B

| | KAF016-070319 | KAF035-070322 |
|---|---|---|
| Chemical Name | | |
| Polyethylene glycol 400 | 95.55 | 90.68 |
| Hydroxypropyl cellulose | 0.49 | 0.47 |
| Steareth 2 | 1.96 | 1.86 |
| Salicylic acid | | 5.00 |
| Miconazole nitrate | 2.00 | 2.00 |
| | 100.00 | 100.00 |
| Propellant* | 8.00 | 8.00 |
| Results | | |
| APPEARANCE: | Good | Good |
| Quality | | |
| Color | White | White |
| Odor | No Odor | No Odor |
| Shakability | Good | Good |
| Collapse time (sec.) | >300 | n/m** |

*16% Propane, 81% Isobutane, 3% N-Butane
**n/m—not measured

### Ex 15b

### Polyethylene Glycol Theoretical Foamable Compositions Comprising PEG 400; Polymer; and an Antifungal Agent and a Keratolytic Agent

Theoretical

| | KAF036-070322 | KAF037-070322 |
|---|---|---|
| Chemical Name | | |
| Polyethylene glycol 400 | 91.65 | 90.67 |
| Hydroxypropyl cellulose | 0.47 | 0.47 |
| Steareth 2 | 1.88 | 1.86 |
| Salicylic acid | 5.00 | 5.00 |
| Ciclopiroxolamine | 1.00 | |
| Terbinafine HCL | | 2.00 |
| | 100.00 | 100.00 |
| Propellant* | 8.00 | 8.00 |

*16% Propane, 81% Isobutane, 3% N-Butane

Note: The liquefied or gas propellant can be added at a concentration of about 3% to about 25%.

### Example 16

### Polyethylene Glycol Foamable Compositions Comprising PEG 400; Polymer; and an Antifungal Agent and Azelaic Acid

| | KAF020- | | |
|---|---|---|---|
| | 070320 | KAF021-070320 | KAF022-070320 |
| Chemical Name | | | |
| Polyethylene | 85.80 | 86.78 | 85.80 |
| glycol 400 | | | |
| Hydroxypropyl | 0.44 | 0.45 | 0.44 |
| cellulose | | | |
| Steareth 2 | 1.76 | 1.78 | 1.76 |
| Azelaic Acid | 10.00 | 10.00 | 10.00 |
| Miconazole nitrate | | | 2.00 |
| Ciclopiroxolamine | | 1.00 | |
| Terbinafine HCL | 2.00 | | |
| | 100.00 | 100.00 | 100.00 |
| Propellant* | 8.00 | 8.00 | 8.00 |
| Results | | | |
| APPEARANCE: | Good | Good | Good |
| Quality | | | |
| Color | White | White | White |
| Odor | No Odor | No Odor | No Odor |
| Shakability | Good | Good | Good |
| Collapse time (sec.) | >300 | >300 | >300 |

*16% Propane, 81% Isobutane, 3% N-Butane

Note: The liquefied or gas propellant can be added at a concentration of about 3% to about 25%.

### Example 17

### Polyethylene Glycol Foamable Compositions Comprising PEG 400; Polymer; and an Antiviral Agent and a Keratolytic Agent

| | KAF031-070322 |
|---|---|
| Chemical Name | |
| Polyethylene glycol 400 | 87.75 |
| Hydroxypropyl cellulose | 0.45 |
| Steareth 2 | 1.80 |
| Acyclovir | 5.00 |
| Salicylic acid | 5.00 |
| | 100.00 |
| Propellant* | 8.00 |
| Results | |
| APPEARANCE: Quality | Good |
| Color | White |
| Odor | No Odor |
| Shakability | Good |

*16% Propane, 81% Isobutane, 3% N-Butane

Note: The liquefied or gas propellant can be added at a concentration of about 3% to about 25%.

### Example 18

### Polyethylene Glycol Foamable Compositions Comprising PEG 400; Polymer; and an Antibiotic Agent and a Keratolytic Agent

| | KAF045-070322 | KAF047-070325 |
|---|---|---|
| Chemical Name | | |
| Polyethylene glycol 400 | 90.68 | 83.85 |
| Hydroxypropyl cellulose | 0.47 | 0.43 |
| Steareth 2 | 1.86 | 1.72 |
| Mupirocine | 2.00 | 2.00 |
| Salicylic acid | 5.00 | |
| Lactic acid | | 12.00 |
| | 100.00 | 100.00 |
| Propellant* | 8.00 | 8.00 |
| Results | | |
| APPEARANCE: Quality | Good | Good |
| Color | White | White |
| Odor | No Odor | No Odor |
| Shakability | Good | Good |

*16% Propane, 81% Isobutane, 3% N-Butane

Note: The liquefied or gas propellant can be added at a concentration of about 3% to about 25%.

Waterless Silicone in Polyethylene Glycol Formulations

### Example 19

### Silicones in Polyethylene Glycol Foamable Compositions Comprising PEG 200/400; ASOS; and an Antiviral Agent and a Keratolytic Agent

| | KAF050-070325 | KAF051-070325 |
|---|---|---|
| Chemical Name | | |
| PEG 400 | 37.20 | 37.20 |
| PEG 200 | 37.20 | 37.20 |
| DIMETHICONE | 2.79 | 2.79 |
| Cyclomethicone | 1.40 | 1.40 |
| ASOS | 2.79 | 2.79 |
| Stearic acid | 8.37 | 8.37 |
| Steareth-2 | 3.26 | 3.26 |
| Miconazole nitrate | 2.00 | |
| Terbinafine HCL | | 2.00 |
| Salicylic acid | 5.00 | 5.00 |
| | 100.00 | 100.00 |
| Propellant* | 8.00 | 8.00 |
| Results | | |
| APPEARANCE: Quality | Good | Good |
| Color | White | White |
| Odor | No Odor | No Odor |
| Shakability | Good | Good |

*16% Propane, 81% Isobutane, 3% N-Butane

Note: the liquefied or gas propellant can be added at a concentration of about 3% to about 25%.

What is claimed is:

1. An augmented anti-infective foamable composition comprising:
a foamable carrier;
a therapeutically safe and effective concentration of an anti-infective agent;
an augmenting agent selected from the group consisting of a keratolytic agent and a skin penetration enhancer; and
a propellant;
wherein the composition is housed in a container and upon release is expandable to form a breakable foam, and
wherein the foamable carrier is selected to generate a foam of good or excellent quality in the presence of the augmenting agent and anti-infective agent.

2. The augmented anti-infective foamable composition of claim 1, wherein propellant comprises a liquefied or compressed gas propellant at a concentration of about 3% to about 25% by weight of the total composition.

3. The augmented anti-infective foamable composition of claim 1, wherein the foamable carrier is selected from the group consisting of oil-in-water emulsions, water-in-oil emulsions, waterless oleaginous formulations, waterless polyethylene glycol and propylene glycol based compositions, waterless silicone in polyethylene glycol based compositions and waterless silicone in propylene glycol based compositions.

4. The augmented anti-infective foamable composition of claim 3, wherein the foamable carrier comprises:
a solvent,
a surface active agent, and
one or more optional agents selected from the group consisting of a one or more optional agents selected from the group comprising a surfactant, co-emulsifier or foam stabilizer; a polymeric agent, a viscosity, bulking or firming agent; a foam adjuvant; a co-solvent; a penetration enhancer, a stabilizer, a modulating agent; a drying agent, and an agent capable of having an occlusive effect.

5. The augmented anti-infective foamable composition of claim 4, wherein the foamable carrier comprises an oil-in-water emulsion comprising:
a hydrophobic solvent, at a concentration of about 2% to about 75% by weight;
about 0.1% to about 5% by weight of a surface-active agent; and
about 0.01% to about 5% by weight of at least one polymeric agent; and
water.

6. The augmented anti-infective foamable composition of claim 4, wherein the foamable carrier comprises a water-in-oil emulsion comprising:
a hydrophobic solvent, at a concentration of about 70% to about 96.5% by weight;
about 0.1% to about 5% by weight of a surface-active agent;
about 0.01% to about 5% by weight of at least one polymeric agent, and water.

7. The augmented anti-infective foamable composition of claim 4, wherein the foamable carrier comprises a waterless oleaginous composition comprising:
a hydrophobic solvent at a concentration of about 70% to about 96.5% by weight;
about 0.1% to about 5% by weight of a surface-active agent; and
about 0.01% to about 5% by weight of at least one polymeric agent.

8. The augmented anti-infective foamable composition of claim 4, wherein the foamable carrier comprises a waterless polyethylene glycol and propylene glycol based compositions comprising:
a solvent comprising a polyethylene glycol, a propylene glycol, or mixtures thereof present at a concentration of about 70% to about 96.5% by weight of the total composition;
about 0.1% to about 5% by weight of a surface-active agent;
about 0.01% to about 5% by weight of at least one polymeric agent.

9. The augmented anti-infective foamable composition of claim 4, wherein the foamable carrier comprises a waterless silicone in propylene glycol or polyethylene glycol based compositions comprising:
a silicone;
a propylene glycol or polyethylene glycol, wherein the glycol is present at a concentration of about 70% to about 96.5% by weight of the total composition;
at least one polymeric agent at a concentration of about 0.1% to about 5% by weight of the total composition, wherein the at least one polymeric agent is selected from a bioadhesive agent, a gelling agent, a film forming agent and a phase change agent; and
at least one surface-active agent at a concentration of about 0.1% to less than about 10% by weight of the total composition.

10. The augmented anti-infective foamable composition of claim 1, wherein the antifungal agent is selected from the group the consisting of an azole, an imidazole, a triazole, a hiadiazole, a pyrazole, an oxazole, a thiazole, a thiadiazole, a thiatriazoles and a benzimidazole, an antifungal peptide, a polyene, an antifungal pyrimidine, an antifungal allylamine, an antifungal morpholine derivative, a thiocarbamate, a sulfonamide, a plant oil or a plant extract possessing antifungal activity, an anti-microbial metal, an oxidizing agent and a cationic antimicrobial agent.

11. The augmented anti-infective foamable composition of claim 1, wherein the augmenting agent is selected from the group consisting of an alpha-hydroxy acid, a short chain carboxylic acid having up to 6 carbon atoms in its skeleton, a di-carboxylic acid, a phenol and a retinoid.

12. The augmented anti-infective foamable composition of claim 1, wherein the augmenting agent is selected from the group consisting of:
a. an alpha-hydroxy acid, selected from the group consisting of agaricic acid, aleuritic acid, allaric acid, altraric acid, arabiraric acid, ascorbic acid, atrolactic acid, benzilic acid, citramalic acid, citric acid, dihydroxytartaric acid, erythraric acid, galactaric acid, galacturonic acid, glucaric acid, glucuronic acid, glyceric acid, glycolic acid, gularic acid, gulonic acid, hydroxypyruvic acid, idaric acid, isocitric acid, lactic acid, lyxaric acid, malic acid, mandelic acid, mannaric acid, methyllacetic acid, mucic acid, phenyllacetic acid, pyruvic acid, quinic acid, ribaric acid, ribonic acid, saccharic acid, talaric acid, tartaric acid, tartronic acid, threaric acid, tropic acid, uronic acids, xylaric acid and analogs, derivatives, esters and salts thereof;
b. urea;
c. salicylic acid;
d. a dicarboxylic acid, selected from the group consisting of malonic acid (propanedioic acid), succinic acid (butanedioic acid), glutaric acid (pentanedioic acid), adipic acid, pimelic acid, suberic acid, azelaic acid, sebacic acid, 1,11-undecanedioic acid, 1,12-dodecanedioic acid, 1,13-tridecanedioic acid, 1,14-tetradecanedioic acid and analogs, derivatives, esters and salts thereof;
e. a retinoid, selected from the group consisting of retinol, retinal, retinoic acid, retinyl acetate, retinyl palmitate, retinyl ascorbate, isotretinoin, tazarotene, adapalene, 13-cis-retinoic acid, acitretin all-trans beta carotene, alpha carotene, lycopene, 9-cis-beta-carotene, lutein and zeaxanthin and analogs, derivatives, esters and salts thereof.

13. The augmented anti-infective foamable composition of claim 11, wherein the augmenting agent includes at least two keratolytic agents.

14. The augmented anti-infective foamable composition of claim 13, wherein the augmenting agent includes at least two agents, from different chemical families, selected from the group consisting of: (1) an alpha-hydroxy acid; (2) a beta-hydroxy acid; (3) a short-chain carboxylic acid; (4) a hydroxyl benzene; (5) a vitamin A derivative; and (6) urea.

15. The augmented anti-infective foamable composition of claim 13, wherein the keratolytic agent consists of at least two keratolytic agents, selected from the group consisting of urea, a hydroxyl acid, a phenol, a dicarboxylic acid and a retinoid.

16. The augmented foamable composition of claim 1, wherein the penetration enhancer is selected from the group consisting of a polyol, glycerol, ethylene glycol, propylene glycol, hexylene glycol, diethylene glycol, propylene glycol n-alkanols, a terpene, a di-terpene, a tri-terpene, a terpen-ol, limonene, 1-menthol, dioxolane, a sulfoxide, dimethylsulfoxide, dimethylformanide, methyl dodecyl sulfoxide, dimethylacetamide, dimethylisosorbide, a monooleate of ethoxylated glyceride, azone, 2-(n-nonyl)-1,3-dioxolane, isopropyl myristate, isopropyl palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; acetamide oleate, triolein, an alkanoic acid, caprylic acid dialkylamino acetate and a polyethylene glycol.

17. The augmented anti-infective foamable composition of claim 1, wherein the antifungal agent is selected from the group consisting of:
a. an azole, selected from the group consisting of azanidazole, bifonazole, butoconazol, chlormidazole, climbazole, cloconazole, clotrimazole, dimetridazole, econazole, enilconazole, fenticonazole, fezatione, fluconazole, flutrimazole, isoconazole, itraconazole, ketoconazole, lanoconazole, metronidazole, metronidazole benzoate, miconazole, neticonazole, nimorazole, niridazole, omoconazol, ornidazole, oxiconazole, posaconazole, propenidazole, ravuconazole, secnidazol, sertaconazole, sulconazole, thiabendazole, tinidazole, tioconazole, voriconazol and analogs, derivatives, esters and salts thereof;
b. a polyene, selected from the group consisting of amphotericin, aureofungin, ayfactin, azalomycin, blasticidin, candicidin, candicidin methyl ester, candimycin, chinopricin, filipin, flavofungin, fradicin, hamycin, hydropricin, levorin, lucensomycin, lucknomycin, mediocidin, mepartricin, methylamphotericin, natamycin, niphimycin, nystatin, oxypricin, partricin, pentamycin, perimycin, pimaricin, primycin, proticin, rimocidin, sistomycosin, sorangicin, trichomycin and analogs, derivatives, esters and salts thereof;
c. an antifungal agent, selected from the group consisting of Terbinafine, naftifine, ciclopirox, ciclopiroxolmine, amorolfine, tolnaftate, flucytosine, mafenide, dapsone, griseofulvin and analogs, derivatives, esters and salts thereof;
d. an oil or an extract of a plant, selected from the group consisting of anise, basil, bergemont, burdock, buchu, chaparral, camphor, cardamom, carrot, canola, cassia, catnip, cedarwood, citronella, clove, couchgrass, cypress, echinacea, eucalyptus, faenia interjecta, garlic, ginger, grapefruit, holy thistle, hops, hyssop, jasmine, jojova, lavender, lavandin, lemon, lime, mandarin, marigold, marjoram, maytenus ilicifolia, maytenus evonymoides, maytenus aquifolia, micromonospora, myrrh, neroli, nutmeg, orange, ordyceps sinensis, peppermint, perilla, petitgrain, plantain, putterlickia verrucosa, putterlickia pyracantha, putterlickia retrospinosa, rosemary, sage, spearmint, star anise, St. John's wort, red clover, tangerine, tea tree, terfezia clayeryi, thyme vanilla, verbena, white clover and yellow dock.
e. an ionic metal ion, or an elemental metal, selected from the group consisting of silver, copper, zinc, mercury, tin, lead, bismuth, gold, cadmium and chromium; and
f. an oxidizing agent, selected from the group consisting of hydrogen peroxide, benzoyl peroxide, a halogen, an oxygenated halogen, a bleaching agent, a hypochlorite a perchlorite, iodine, an iodate and an organic oxidizing agent.

18. The augmented anti-infective foamable composition of claim 1, wherein the antifungal agent is selected from the group consisting of an azole, an allylamine and ciclopiroxolamine; and the keratolytic agent is selected from the group consisting of urea, a hydroxyl acid, a phenol, a dicarboxylic acid and a retinoid.

19. The augmented anti-infective foamable composition of claim 1, wherein the antifungal agent comprises terbinafine; and the keratolytic agent comprises urea.

20. The augmented anti-infective foamable composition of claim 19, wherein the concentration of terbinafine is in the range of 0.5% and 4% and the concentration of urea is in the range of 2% and 40%.

21. The augmented anti-infective foamable composition of claim 1, wherein the antifungal agent comprises an azole; and the keratolytic agent comprises a retinoid.

22. The augmented anti-infective foamable composition of claim 1, wherein the antifungal agent comprises ciclopiroxolamine; and the keratolytic agent comprises selected an ester of a fatty acid.

23. The augmented anti-infective foamable composition of claim 1, wherein the antibiotic agent is selected from the group consisting of a beta-lactam antibiotic, an aminoglycoside, an ansa-type antibiotic, an anthraquinone, an azole, an antibiotic glycopeptide, a macrolide, an antibiotic nucleoside, an antibiotic peptide, an antibiotic polyene, an antibiotic polyether, an antibiotic quinolone, an antibiotic steroid, a sulfonamide, an antibiotic metal, an oxidizing agent, a periodate, a hypochlorite, a permanganate, a substance that release free radicals and/or active oxygen, a cationic antimicrobial agent, a quaternary ammonium compound, a biguanide, a triguanide, a bisbiguanide, a polymeric biguanide, and analogs, derivatives, salts, ions and complexes thereof.

24. The augmented anti-infective foamable composition of claim 1, wherein the antiviral agent is selected from the group consisting of acyclovir, gancyclovir, ribavirin, amantadine, rimantadine nucleoside-analog reverse transcriptase inhibitors, such as zidovudine, didanosine, zalcitabine, tavudine, lamivudine and vidarabine, non-nucleoside reverse transcriptase inhibitors, such as nevirapine and delavirdine, protease inhibitors, such as saquinavir, ritonavir, indinavir and nelfinavir, and interferons and derivatives, esters, salts and mixtures thereof.

25. The augmented anti-infective foambable composition of claim 5, 6 or 7, wherein the hydrophobic solvent is selected from the group consisting of mineral oil, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, unsaturated or polyunsaturated oils, such as olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oil, and a silicone oil.

26. The augmented anti-infective foambable composition of claim 5, wherein the emulsion is an oil-in-water emulsion; and wherein the surface-active agent is a non-ionic surfactant, having an HLB value between about 9 and about 14.

27. The augmented anti-infective foambable composition of claim 6, wherein the emulsion is a water-in-oil emulsion; and wherein the surface-active agent is a non-ionic surfactant, having an HLB value between 2 and about 9.

28. The augmented anti-infective foambable composition of claim 4, wherein the surface-active agent is a non-ionic surfactant.

29. The augmented anti-infective foambable composition of claim 28, wherein the surface-active agent is a non-ionic surfactant, selected from the group consisting of a polysorbate, a poly(oxyethylene) (POE) fatty acid ester, a poly(oxyethylene) alkylyl ether, a polyethylene glycol cetyl ether a sucrose ester, an ester of sorbitol, a mono or diglycerides, and a sucrose ester.

30. The augmented anti-infective foambable composition of claim 4, wherein the surface-active agent is a combination of at least one non-ionic surfactant and at least one ionic surfactant; and wherein the ratio between the non-ionic surfactant and the ionic surfactant is selected 100:1 to 6:1; and 1:1 and 20:1.

31. The augmented anti-infective foambable composition of claim 4, wherein the concentration of the polymeric agent is selected such that the viscosity of the composition, prior to filling of the composition into aerosol canisters, is less than 12,000 CPs.

32. The augmented anti-infective foambable composition of claim 4, further including about 0.1% to about 5% by weight of a foam adjuvant is selected from the group consisting of a fatty alcohol having 15 or more carbons in their carbon chain; a fatty acid having 16 or more carbons in their carbon chain; fatty alcohols, derived from beeswax and including a mixture of alcohols, a majority of which has at least 20 carbon atoms in their carbon chain; a fatty alcohol having at least one double bond; a fatty acid having at least one double bond; a branched fatty alcohol; a branched fatty acid; a fatty acid substituted with a hydroxyl group; cetyl alcohol; stearyl alcohol; arachidyl alcohol; behenyl alcohol; 1-triacontanol; hexadecanoic acid; stearic acid; arachidic acid; behenic acid; octacosanoic acid; 12-hydroxy stearic acid and mixtures thereof.

33. The augmented anti-infective foambable composition of claim 1, wherein the foamable composition further comprises at least one additional active agent.

34. The augmented anti-infective foambable composition of claim 33, wherein the additional active agent is selected from the group consisting of active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin A, vitamin A derivatives, vitamin B, vitamin B derivative, vitamin C, vitamin C derivatives, vitamin D, vitamin D derivatives, vitamin E, vitamin E derivatives, vitamin F, vitamin F derivatives, vitamin K, vitamin K derivatives, wound healing agents and wart removers.

35. The augmented anti-infective foambable composition of claim 1, wherein the anti-infective agent is formulated for slow release.

36. The augmented anti-infective foamable composition of claim 1, wherein the compositions includes an anti-infective agent and augmenting agent combination selected from the group consisting of:
urea and terbinafine; urea and miconazole; urea and ciclopiroxolamine; urea and itraconazole; urea and clindamycin; urea and acyclovir; urea and mupirocine; retanoic acid and; retanoic acid and miconazole; retanoic acid and ciclopiroxolamine; retanoic acid and itraconazole; retanoic acid and clindamycin; retinoic acid and acyclovir; retinoic acid and mupirocine; azelaic acid and terbinafine; azelaic acid and miconazole; azelaic acid and ciclopiroxolamine; azelaic acid and itraconazole; azelaic acid and clindamycin; azelaic acid and acyclovir; azelaic acid and mupirocine; salicylic acid and terbinafine; salicylic acid and miconazole; salicylic acid and ciclopiroxolamine; salicylic acid and itraconazole; salicylic acid and clindamycin; salicylic acid and acylclovir; salicylic acid and mupirocine; lactic acid and terbinafine; lactic acid and miconazole; lactic acid and ciclopiroxolamine; lactic acid and itraconazole; lactic acid and clindamycin; lactic acid and acyclovir; and lactic acid and mupirocine.

37. A method of treating, alleviating or preventing a disorder of the skin, a body cavity or mucosal surface, wherein the disorder involves a fungal, bacterial or viral infection as one of its etiological factors, including:
administering topically to a subject having the disorder, a foamed composition by releasing a foamable composition from a container to form a breakable foam,
wherein the foamable composition comprises:
a foamable carrier;
a therapeutically safe and effective concentration of an anti-infective agent;
an augmenting agent selected from the group consisting of a keratolytic agent and a skin penetration enhancer; and
a propellant;
wherein the foamable carrier is selected to generate a foam of good or excellent quality in the presence of the augmenting agent and anti-infective agent.

38. A method of treatment according to claim 37, wherein the foamable carrier is selected from the group consisting of oil-in-water emulsions, water-in-oil emulsions, waterless oleaginous formulations, waterless polyethylene glycol and propylene glycol based compositions, waterless silicone in polyethylene glycol based compositions and waterless silicone in propylene glycol based compositions.

39. A method of treatment according to claim 38, wherein the foamable carrier comprises:
a solvent,
a surface active agent, and
one or more optional agents selected from the group consisting of a one or more optional agents selected from the group comprising a surfactant, co-emulsifier or foam stabilizer; a polymeric agent, a viscosity, bulking or firming agent; a foam adjuvant; a co-solvent; a penetration enhancer, a stabilizer, a modulating agent; a drying agent, and an agent capable of having an occlusive effect.

40. A method of treatment according to claim 37, wherein the antifungal agent is selected from the group consisting of an azole, an imidazole, a triazole, a hiadiazole, a pyrazole, an oxazole, a thiazole, a thiadiazole, a thiatriazoles and a benzimidazole, an antifungal peptide, a polyene, an antifungal pyrimidine, an antifungal allylamine, an antifungal morpholine derivative, a thiocarbamate, a sulfonamide, a plant oil or a plant extract possessing antifungal activity, an anti-microbial metal, an oxidizing agent and a cationic antimicrobial agent.

41. A method of treatment according to claim 37, wherein the antibiotic agent is selected from the group consisting of a beta-lactam antibiotic, an aminoglycoside, an ansa-type antibiotic, an anthraquinone, an azole, an antibiotic glycopeptide, a macrolide, an antibiotic nucleoside, an antibiotic peptide, an antibiotic polyene, an antibiotic polyether, an antibiotic quinolone, an antibiotic steroid, a sulfonamide, an antibiotic metal, an oxidizing agent, a periodate, a hypochlorite, a permanganate, a substance that release free radicals and/or active oxygen, a cationic antimicrobial agent, a quaternary ammonium compound, a biguanide, a triguanide, a bisbiguanide, a polymeric biguanide, and analogs, derivatives, salts, ions and complexes thereof.

42. A method of treatment according to claim 37, wherein the antiviral agent is selected from the group consisting of acyclovir, gancyclovir, ribavirin, amantadine, rimantadine nucleoside-analog reverse transcriptase inhibitors, such as zidovudine, didanosine, zalcitabine, tavudine, lamivudine and vidarabine, non-nucleoside reverse transcriptase inhibitors, such as nevirapine and delavirdine, protease inhibitors, such as saquinavir, ritonavir, indinavir and nelfinavir, and interferons and derivatives, esters, salts and mixtures thereof.

43. A method of treatment according to claim 37, wherein the keratolytic agent is selected from the group consisting of an alpha-hydroxy acid, a short chain carboxylic acid, having up to 6 carbon atoms in its skeleton, a di-carboxylic acid, a phenol and a retinoid.

44. A method of treatment according to claim 37, wherein the antifungal agent is selected from the group consisting of an azole, an allylamine and ciclopiroxolamine; and the keratolytic agent is selected from the group consisting of urea, a hydroxyl acid, a phenol, a dicarboxylic acid and a retinoid.

45. A method of treatment according to claim 44, wherein the antifungal agent is terbinafine at a concentration in the range of 0.5% and 4%; and the keratolytic agent is urea at a concentration of 2% and 40%.

46. A method of treatment according to claim 37, wherein the keratolytic agent consists of at least two keratolytic agents, selected from the group consisting of urea, a hydroxyl acid, a phenol, a dicarboxylic acid and a retinoid.

47. A method of treatment according to claim 37, wherein the antifungal agent comprises ciclopiroxolamine; and the keratolytic agent comprises an ester of a fatty acid.

48. A method of treatment according to claim 37, wherein the foamable composition further comprises at least one additional active agent.

49. A method of treatment according to claim 37, wherein the foamable composition further comprises at least two active agent.

50. A method of treat according to claim 49, wherein at least one active agent is directed to treating a primary infection and at least one active agent is directed to treating a secondary infection.

51. A method of treatment according to claim 37, wherein the disorder is selected from the group consisting of a dermatose, a dermatitis, a vaginal disorder, a vulvar disorder, an anal disorder, a disorder of a body cavity, an ear disorder, a disorder of the nose, a disorder of the respiratory system, dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, ecthyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, grannuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo, burn, wound, cut, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, osteoarthritis, joint pain, hormonal disorder, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

52. An augmented anti-infection kit for use in the treatment of an infection comprising:
a first anti-infective foamable composition contained in a first pressurized container, said first anti-infective foamable composition comprising a therapeutically safe and effective concentration of a first anti-infective agent and a first augmenting agent selected from the group consisting of a keratolytic agent and a skin penetration enhancer in a first foamable carrier, and
a second anti-infective foamable composition contained in a second pressurized container, said second anti-infective foamable composition comprising a therapeutically safe and effective concentration of a second anti-infective agent; and an optional second augmenting agent selected from the group consisting of a keratolytic agent and a skin penetration enhancer in a second foamable carrier,
wherein at least one of the foamable carrier, augmenting agents or anti-infective agents differ between the first and second anti-infective foamable compositions.

53. The kit of claim 49, wherein the first and second foamable carriers are independently selected from the group consisting of oil-in-water emulsions, water-in-oil emulsions, waterless oleaginous formulations, waterless polyethylene glycol and propylene glycol based compositions, waterless silicone in polyethylene glycol based compositions and waterless silicone in propylene glycol based compositions.

54. The kit of claim 49, wherein the first and second anti-infective agents are different.

55. The kit of claim 51, wherein the different anti-infective agents are selected to treat different infections.

56. The kit of claim 55, wherein at least one infection is a primary infection and at least one infection is a secondary infection.

57. The kit of claim 52, wherein the first and second foamable carriers are different.

58. The kit of claim 54, wherein the first anti-infective agent is stable at a first foam condition and the first foamable carrier is selected to provide a first foam condition; and the second anti-infective agent is stable at a second foam condition and the second foamable carrier is selected to provide a second foam condition.

59. The kit of claim 54, wherein said foam condition is selected from the group consisting of pH, water stability and water solubility.

60. The kit of claim 59, wherein first and second foamable compositions are set at or buffered at different pHs.

61. The kit of claim 52, further comprising a dispensing head, said dispensing head in flow communication with the first and second pressurized containers for dispensing said first and second anti-infective foamable compositions.

62. The kit of claim 61, wherein the dispensing head is positioned and arranged to dispense said first and second anti-infective foamable compositions sequentially.

63. The kit of claim 61, wherein the dispensing head is positioned and arranged to dispense said first and second anti-infective foamable compositions simultaneously.

64. An augmented foamable composition comprising:
a foamable carrier;
an augmenting agent selected from the group consisting of a keratolytic agent and a skin penetration enhancer; and
a propellant;
wherein the composition is housed in a container and upon release is expandable to form a breakable foam, and
wherein the foamable carrier is selected to generate a foam of ‘good’ or ‘excellent’ quality in the presence of the augmenting agent and an anti-infective agent.
